SEQUENCE LISTING (14 sequences)

SEQ ID NO: 1 - DNA, 95 bp, synthetic construct
  source 1..95: mol_type = other DNA
ccttggcacccgagaattccgcatctaactgctgcgccgccgggaagaactgtacggttagatgcaaaaaaaaaaaaaaaaaaaaaaaaaaaaaa

SEQ ID NO: 2 - DNA, 100 bp, synthetic construct
  source 1..100: mol_type = other DNA
ccttggcacccgagaattcccagcagcgtaaagggggtgtttgtgcggtgtggagtgcgcgtgctgctggaaaaaaaaaaaaaaaaaaaaaaaaaaaaaa

SEQ ID NO: 3 - DNA, 100 bp, synthetic construct
  source 1..100: mol_type = other DNA
ccttggcacccgagaattccatcaggctggatggtagctcggtcggggtgggtgggttggcaagtctgataaaaaaaaaaaaaaaaaaaaaaaaaaaaaa

SEQ ID NO: 4 - DNA, 98 bp, synthetic construct
  source 1..98: mol_type = other DNA
ccttggcacccgagaattccgccattgccattgccattgccattgccattgccattgccattgccattaaaaaaaaaaaaaaaaaaaaaaaaaaaaaa

SEQ ID NO: 5 - DNA, 99 bp, synthetic construct
  source 1..99: mol_type = other DNA
ccttggcacccgagaattccccaataccgcggggtgggtctagtgtggatgtttagggggcggtattggaaaaaaaaaaaaaaaaaaaaaaaaaaaaaa

SEQ ID NO: 6 - DNA, 83 bp, synthetic construct
  source 1..83: mol_type = other DNA
ccttggcacccgagaattcctacaggttctggggggtgggtggggaacctgttaaaaaaaaaaaaaaaaaaaaaaaaaaaaaa

SEQ ID NO: 7 - DNA, 86 bp, synthetic construct
  source 1..86: mol_type = other DNA
ccttggcacccgagaattccctaaccccgggtgtggtgggtgggcaggggggttagaaaaaaaaaaaaaaaaaaaaaaaaaaaaaa

SEQ ID NO: 8 - DNA, 92 bp, synthetic construct
  source 1..92: mol_type = other DNA
ccttggcacccgagaattcctcgccgcgtctttatggctggggatggtgtgggttgcggcgaaaaaaaaaaaaaaaaaaaaaaaaaaaaaaa

SEQ ID NO: 9 - DNA, 100 bp, synthetic construct
  source 1..100: mol_type = other DNA
ccttggcacccgagaattcccttattcaattcctgtgggaaggctatagaggggccagtctatgaataagaaaaaaaaaaaaaaaaaaaaaaaaaaaaaa

SEQ ID NO: 10 - DNA, 96 bp, synthetic construct
  source 1..96: mol_type = other DNA
ccttggcacccgagaattcccgcgccgtactagatgcaacccgactactaacgtcgtacgagcgcgaaaaaaaaaaaaaaaaaaaaaaaaaaaaaa

SEQ ID NO: 11 - DNA, 98 bp, synthetic construct
  source 1..98: mol_type = other DNA
ccttggcacccgagaattcccactacagaggttgcgtctgtcccacgttgtcatggggggttggcctgaaaaaaaaaaaaaaaaaaaaaaaaaaaaaa

SEQ ID NO: 12 - DNA, 100 bp, synthetic construct
  source 1..100: mol_type = other DNA
ccttggcacccgagaattcctgcaacggggggagcggatgtctgggaaaaccgcggggtgccctccgagcaaaaaaaaaaaaaaaaaaaaaaaaaaaaaa

SEQ ID NO: 13 - DNA, 100 bp, synthetic construct
  source 1..100: mol_type = other DNA
ccttggcacccgagaattccccagagtgacgcagcaccaccaccgtacaattttttcattacctactcggaaaaaaaaaaaaaaaaaaaaaaaaaaaaaa

SEQ ID NO: 14 - DNA, 97 bp, synthetic construct
  source 1..97: mol_type = other DNA
ccttggcacccgagaattccaacaccgtggaggatagttcggtggctgttcagggtctcctcccggtgaaaaaaaaaaaaaaaaaaaaaaaaaaaaa

(19) United States
(12) Patent Application Publication
(10) Pub. No.: US 2026/0297566 A1
(43) Pub. Date: Oct. 1, 2026

(54) CELLULAR ANALYSIS USING BINDING AGENTS

(71) Applicant: Cellanome, Inc., Foster City, CA (US)

(72) Inventors: Mostafa RONAGHI, Atherton, CA (US); David PORCIANI, San Diego, CA (US)

(21) Appl. No.: 19/633,485

(22) Filed: Mar. 30, 2026

Related U.S. Application Data
(60) Provisional application No. 63/781,195, filed on Mar. 31, 2025.

(51) Int. Cl. C12N 15/10

(52) U.S. Cl. CPC C12N 15/1065

(57) ABSTRACT

Disclosed herein are methods for analyzing cellular surfaceomic and secretomic analysis using aptamers, as well as composition, systems, and devices for performing such methods. The methods can include combining aptamers with a cell, wherein a first subset of the aptamers couple to the cell and a second subset of the aptamers do not couple to the cell, hybridizing capturable sequences of the first subset of the aptamers to nucleic acid barcodes, extending the nucleic acid barcodes and/or aptamers, and sequencing the extended nucleic acid barcodes and/or extended aptamers.

### CROSS REFERENCE

This application claims the benefit of U.S. Provisional Application No. 63/781,195 filed Mar. 31, 2025, which is incorporated herein by reference in its entirety.

### SEQUENCE LISTING

The instant application contains a Sequence Listing which has been submitted electronically in XML format and is hereby incorporated by reference in its entirety. Said XML copy, created on Mar. 25, 2026, is named 59528-736_201_SL.xml and is 14,196 bytes in size.

### BACKGROUND

Cell surfaceomic profiles can reflect type, state, activity, health and function of a cell. Small changes in intracellular biochemistry can affect pronounced, measurable changes in surfaceomic composition and organization that are often indiscernible through mRNA profiling. Even among clonally prepared populations, cell surfaceomes can exhibit heterogeneity that may be difficult to analyze through bulk measurements. In addition, single-cell surfaceomics are challenged by the density, complexity, and throughput requirements.

### SUMMARY

In some embodiments, the present disclosure provides a method for cellular analysis comprising: combining aptamers with a cell, wherein a first subset of the aptamers couple to the cell and a second subset of the aptamers do not couple to the cell; separating the second subset of the aptamers from the cell; inputting the cell and the first subset of the aptamers into a fluidic device, wherein the fluidic device comprises nucleic acid barcodes; hybridizing capturable sequences of the first subset of the aptamers to the nucleic acid barcodes; extending the nucleic acid barcodes to generate extended nucleic acid barcodes or extending the first subset of the aptamers to generate extended aptamers; eluting the extended nucleic acid barcodes or the extended aptamers from the fluidic device; and sequencing the extended nucleic acid barcodes or the extended aptamers.

In one aspect, the method further comprises associating the first subset of the aptamers with an identity for one or more surface proteins on the cell. In another aspect, the method further comprises identifying the cell based on the sequencing of the first subset of the aptamers. In a further aspect, the method further comprises, before the hybridizing, dissociating the first subset of the aptamers from the cell. In certain aspects, the dissociating comprises: i) lysing the cell, ii) reducing an ionic strength of a solution surrounding the cell, iii) increasing or reducing a pH of the solution surrounding the cell, iv) inputting a chaotropic agent into the fluidic device, v) inputting an organic solvent into the fluidic device, vi) increasing a temperature of the fluidic device, or vii) a combination thereof.

In some aspects, the separating comprises removing the second subset of the aptamers from the fluidic device in one or more wash steps, wherein the first subset of the aptamers remain coupled to the cell during the one or more wash steps. In some such cases, the wash steps are subsequent to the synthesizing. In additional aspects, the separating comprises, prior to the inputting, centrifuging a composition comprising the cell and the aptamers, thereby generating a supernatant comprising the second subset of the aptamers and a pellet comprising the cell and the first subset of the aptamers coupled to the cell, and separating the supernatant from the pellet. In further aspects, the separating comprises, prior to the inputting, coupling a magnetic particle to the cell, wherein the cell is coupled to the first subset of the aptamers, and magnetically separating the magnetic particle coupled to the cell from the second subset of the aptamers. In certain aspects, the separating comprises, prior to the inputting, filtering a composition comprising the cell and the aptamers, thereby generating a filtrate comprising the second subset of the aptamers and a residue comprising the cell and the first subset of the aptamers coupled to the cell.

In some aspects, the combining and the separating are prior to the inputting. In other aspects, the combining and the separating are subsequent to the inputting, the aptamers are input into the fluidic device, and the separating comprises removing the second subset of the aptamers from the fluidic device. In further aspects, the inputting is subsequent to the combining and prior to the separating, the aptamers are input into the fluidic device along with the cell, and the separating comprises removing the second subset of the aptamers from the fluidic device.

In a certain aspect, the aptamers comprise: i) the capturable sequences at 3′ ends of the aptamers, and ii) primers at 5′ ends of the aptamers. In an additional aspect, the aptamers further comprise aptamer identity barcodes, wherein the aptamer identity barcodes identify the aptamers to which they are coupled.

In another aspect, the nucleic acid barcodes comprise: i) spatial barcode sequences associated with locations of the nucleic acid barcodes on a surface of the fluidic device, ii) unique molecular identifier sequences, iii) oligonucleotide capture sequences, iv) chemically cleavable moieties, v) enzymatically cleavable moieties, vi) primer binding sites, or vii) a combination thereof. In a particular aspect, the sequencing comprises sequencing the spatial barcode sequences or complements of the spatial barcode sequences, and wherein the method further comprises associating the first subset of the aptamers with the cell based on the spatial barcode sequences or complements of the spatial barcode sequences. In an additional aspect, the oligonucleotide capture sequences comprise polyT sequences, and wherein poly A sequences of the capturable sequences hybridize to the polyT sequences. In one aspect, the sequencing comprises quantifying aptamers of the first subset of the aptamers.

In some aspects, the method further comprises synthesizing one or more chambers that co-enclose the cell, the first subset of aptamers coupled to the cell, and one or more of the nucleic acid barcodes, wherein the nucleic acid barcodes are coupled to a surface of the fluidic device. In some such aspects, the method further comprises degrading the one or more chambers. In another aspect, the method further comprises dissociating the first subset of the aptamers from the cell subsequent to the synthesizing. In a particular aspect, the separating is subsequent to the synthesizing, wherein the separating comprises flowing a liquid into the fluidic device causing the second subset of the aptamers in the one or more chambers to flow out of the one or more chambers through a pore in a wall of the one or more chambers while retaining the cell in the one or more chambers, wherein the cell is larger than the pore in the wall of the one or more chambers.

In an additional aspect, the method further comprises analyzing cellular nucleic acids from the cell, the analyzing comprising: releasing the cellular nucleic acids from the cell; capturing the cellular nucleic acids or fragments of the cellular nucleic acids on additional nucleic acid barcodes coupled to the surface of the fluidic device; and i) extending the additional nucleic acid barcodes using the cellular nucleic acids as templates and sequencing the extended additional nucleic acid barcodes, ii) extending the cellular nucleic acids using the additional nucleic acid barcodes as templates and sequencing the extended cellular nucleic acids, or iii) a combination thereof. In certain aspects, the cellular nucleic acids from the cell comprise mRNA. In further cases, the cellular nucleic acids from the cell comprise guide RNA, wherein the guide RNA comprises a reporter sequence corresponding to a genetic edit for generating a surface protein from the cell, the method further comprises associating the aptamer coupled to the cell with an identity of the surface protein for the cell.

In further aspects, the combining the aptamers with the cell is in a container, and the separating comprises removing the second subset of the aptamers from the container.

In some aspects, the aptamers comprise aptamer binding portions, wherein the aptamer binding portions of the first subset of aptamers couple to the cell, and wherein the aptamer binding portions comprise about 30 to about 70 nucleotides. In further aspects, the aptamer binding portions comprise secondary structure, tertiary structure, or a combination thereof. In certain aspects, the aptamers further comprise first primers and second primers, wherein the first primers and the second primers are disposed on opposite sides of the aptamer binding portions.

In particular aspects, the extending comprises a reverse transcriptase or a DNA polymerase, and wherein the reverse transcriptase or the DNA polymerase comprises strand-displacing activity. In select aspects, the reverse transcriptase comprises Moloney murine leukemia virus reverse transcriptase, avian myeloblastosis virus reverse transcriptase, Thermostable Group II Intron Reverse Transcriptase, or a combination thereof. In particular aspects, the DNA polymerase comprises Bst DNA polymerase, Phi29 DNA polymerase, E. coli DNA polymerase I, or a combination thereof. In additional aspects, the extending comprises a temperature of between about 25° C. and about 45° C.

An additional embodiment disclosed herein provides a method for identifying aptamer binding targets comprising: inputting one or more cells into a fluidic device, wherein the fluidic device comprises a first surface, a second surface, and an array of discrete spots on the first surface, wherein each discrete spot of the array comprises an aptamer configured to couple to a surface protein on a cell of the one or more cells; coupling the surface proteins on the cells to the aptamer for at least one site of the array; synthesizing chambers, wherein each chamber co-encloses the cell, the aptamer coupled to the surface protein on one or more of the cells, and a nucleic acid barcode, wherein the nucleic acid barcode is coupled to i) the first surface at the at least one discrete spot of the array or ii) the second surface at a location of the fluidic device coincident with each site of the array, and wherein the nucleic acid barcode comprises: i) one or more spatial barcode sequences associated with a location of the nucleic acid barcode in the fluidic device, and ii) capture sequences configured to hybridize to cellular nucleic acids from the cell; releasing the cellular nucleic acids from the cell; capturing the cellular nucleic acids on the nucleic acid barcodes, and i) extending the nucleic acid barcodes using the cellular nucleic acids as templates, and sequencing the extended nucleic acid barcodes, ii) extending the cellular nucleic acids using the nucleic acid barcodes as templates, and sequencing the extended cellular nucleic acids, or iii) a combination thereof; and associating the aptamer with the surface proteins on the cells based on the sequencing.

In certain aspects, the aptamers are based on the first subset of aptamers of claim 1. In further aspects, the cellular nucleic acids comprise mRNA molecules that encode the surface proteins or comprise gRNA molecules that comprise barcode sequences associated with an identity of the surface proteins. In additional aspects, the releasing comprises lysing the cells. In particular aspects, the nucleic acid barcodes further comprise unique molecular identifier sequences. In additional select aspects, the one or more cells comprise a protein expression library.

In a particular aspect, the aptamers comprise: i) spatial barcode sequences associated with locations of the aptamers in the fluidic device; ii) unique molecular identifier sequences; iii) barcode sequences associated with nucleic acid sequences of the aptamers; iv) chemically cleavable moieties; v) enzymatically cleavable moieties; vi) primer binding sites; or vii) a combination thereof. In a certain aspect, the aptamers are hybridized to a subset of the nucleic acid barcodes. In another aspect, poly A sequences on the cellular nucleic acids hybridize to polyT capture sequences on the nucleic acid barcodes. In a select aspect, the nucleic acid barcode is at a location of the fluidic device on the second surface coincident with each discrete spot of the array so that the nucleic acid barcode on the second surface is in an opposing relationship with a corresponding discrete spot of the array on the first surface.

Further embodiments of the present disclosure provide a method for cellular analysis comprising: inputting a cell into a fluidic device, wherein the fluidic device comprises an aptamer, wherein the aptamer is coupled to the fluidic device, and wherein the cell releases a protein; coupling the protein to the aptamer; and detecting the protein coupled to the aptamer, wherein the detecting comprises coupling a binding agent to the protein coupled to the aptamer, and detecting a detectable moiety coupled to the binding agent.

In one aspect, the protein is a cytokine. In another aspect, the protein is an intracellular protein, and wherein the cell releases the protein following lysis of the cell. In a certain aspect, the binding agent comprises an antibody or an additional aptamer configured to couple to the protein at a portion different than the aptamer, the detectable moiety comprises a dye, and the detecting comprises collecting an optical signal from the dye.

In an additional aspect, the detectable moiety comprises a nucleic acid sequence of an oligonucleotide coupled to the binding agent, and wherein the detecting comprises: releasing the oligonucleotide from the binding agent; capturing the oligonucleotide on a nucleic acid barcode coupled to the surface of the fluidic device; and extending the nucleic acid barcode using the oligonucleotide as a template and sequencing the extended nucleic acid barcode, extending the oligonucleotide using the nucleic acid barcode as a template and sequencing the extended oligonucleotide, or a combination thereof. In a further aspect, the detectable moiety comprises a nucleic acid sequence of an oligonucleotide coupled to the binding agent, and wherein the detecting comprises detecting the nucleic acid sequence of the oligonucleotide.

An additional embodiment of the present disclosure provides a method for cellular analysis comprising: combining aptamers with a cell, wherein a first subset of the aptamers couple to the cell and a second subset of the aptamers do not couple to the cell; separating the second subset of the aptamers from the cell; forming a droplet, wherein the droplet comprises the cell and nucleic acid barcodes, and wherein the cell is coupled to the first subset of the aptamers; hybridizing first barcode sequences of the first subset of the aptamers to the nucleic acid barcodes; extending the nucleic acid barcodes to generate extended nucleic acid barcodes or extending the first subset of the aptamers to generate extended aptamers; collecting the extended nucleic acid barcodes or the extended aptamers from the droplet; and sequencing the extended nucleic acid barcodes or the extended aptamers.

In some aspects, the nucleic acid barcodes are coupled to a bead, wherein the bead is contained within the droplet. In further aspects, the nucleic acid barcodes comprise: barcode sequences associated with the droplet, unique molecular identifier sequences, oligonucleotide capture sequences, or a combination thereof.

In particular aspects, the cell is the only cell in the droplet. In additional aspects, the forming the droplet comprises: flowing an aqueous solution containing the cell coupled to the aptamer and the nucleic acid barcodes coupled a bead; and flowing an oil phase, wherein the flowed aqueous solution and the flowed oil phase meet at a junction to form the droplet, wherein the droplet comprises the aqueous solution surrounded by the oil phase.

A further embodiment of the present disclosure provides a fluidic device comprising a surface comprising a plurality of discrete spots, wherein each discrete spot of the plurality of discrete spots comprises: nucleic acid barcodes that comprise (i) spatial barcode sequences associated with the discrete spot and (ii) nucleic acid capture sequences, and aptamers. In some aspects, each discrete spot of the plurality of discrete spots comprises a single type of aptamer.

Another embodiment disclosed herein provides a kit comprising: i) a fluidic device comprising a surface comprising a plurality of discrete spots, wherein each discrete spot of the plurality of discrete spots comprises (i) nucleic acid barcodes that comprise a spatial barcode sequence associated with the discrete spot and (ii) a nucleic acid capture sequence; and ii) a plurality of aptamers, wherein each aptamer of the plurality of aptamers comprises a nucleic acid sequence complementary to a nucleic acid capture sequence of the plurality of discrete spots.

In an additional embodiment, the present disclosure provides a method for identifying cell-binding aptamers, comprising: combining aptamers with cells in a container, wherein each aptamer of the aptamers comprises: i) a first primer, ii) an aptamer binding portion, and ii) a second primer, wherein the first primer and the second primer are both coupled to the aptamer binding portion; wherein a first subset of the aptamers couple to the cells and a second subset of the aptamers do not couple to the cells; separating the second subset of the aptamers from the container; dissociating the first subset of aptamers from the cells, and sequencing the first subset of aptamers, thereby identifying the cell-binding aptamers.

In one aspect, the cells comprise 1 to 10 types of human cells. In another aspect, the aptamers comprise 10¹¹ to 10¹⁶ unique aptamer sequences. In an additional aspect, each unique aptamer sequence of a sub-set of the unique aptamer sequences is configured to bind to a unique epitope of a surface protein of a cell. In a further aspect, the first primer and the second primer are both coupled to opposing ends of the aptamer binding portion. In a particular aspect, the separating comprises: i) removing the second subset of the aptamers from the container in one or more wash steps, wherein the first subset of the aptamers remain coupled to the cell during the one or more wash steps; ii) centrifuging the container, thereby generating a supernatant comprising the second subset of the aptamers and a pellet comprising the cells and the first subset of the aptamers coupled to the cells, and separating the supernatant from the pellet; iii) coupling a magnetic particle to a cell of the cells, wherein the cell is coupled to the first subset of the aptamers, and magnetically separating the magnetic particle coupled to the cell from the second subset of the aptamers; or iv) filtering a liquid in the container, thereby generating a filtrate comprising the second subset of the aptamers and a residue comprising the cell and the first subset of the aptamers coupled to the cells.

An additional embodiment of the present disclosure provides a fluidic device, comprising: one or more chambers, wherein a first chamber of the one or more chambers comprises a first aptamer configured to bind to a first cell or a component of the first cell, wherein a second chamber of the one or more chambers comprises a second aptamer configured to bind to a second cell or a component of the second cell, wherein the first aptamer is different from the second aptamer; and one or more nucleic acid barcodes disposed on a planar surface of the fluidic device, wherein the one or more nucleic acid barcodes comprise one or more capture sequences configured to hybridize to one or more cellular nucleic acids from the first cell or the second cell.

In some aspects, the one or more chambers comprise one or more polymer matrix walls. In other aspects, the first aptamer is configured to bind to the component of the first cell, and wherein the component of the first cell comprises a surface protein of the first cell. In further aspects, the one or more nucleic acid barcodes further comprise one or more spatial barcode sequences associated with locations of the one or more nucleic acid barcodes on the planar surface of the fluidic device. In additional aspects, the one or more nucleic acid barcodes further comprise one or more unique molecular identifier (UMI) sequences. In particular aspects, the one or more cellular nucleic acids from the first cell or the second cell comprise mRNA molecules that encode surface proteins. In certain aspects, the one or more cellular nucleic acids from the first cell or the second cell comprise guide RNA (gRNA) molecules that comprise barcode sequences associated with an identity of a surface protein of the first cell or the second cell. In select aspects, the one or more capture sequences comprise a polyT capture sequence configured to hybridize to poly A sequences on the one or more cellular nucleic acids from the first cell or the second cell.

In a particular aspect, the first aptamer or the second aptamer is disposed on the planar surface of the fluidic device. In another aspect, the first aptamer or the second aptamer is disposed on an additional surface of the fluidic device, wherein the additional surface is located opposite of the planar surface. In an additional aspect, the first chamber comprises a first subset of nucleic acid barcodes of the one or more nucleic acid barcodes, and wherein the second chamber comprises a second subset of nucleic acid barcodes of the one or more nucleic acid barcodes. In one particular aspect, the first subset of nucleic acid barcodes comprise a first spatial barcode sequence, wherein the second subset of nucleic acid barcodes comprise a second spatial barcode sequence, and wherein the first spatial barcode sequence is different than the second spatial barcode sequence. In a further aspect, the first aptamer is disposed on the planar surface of the fluidic device adjacent to the first subset of nucleic acid barcodes, and wherein the second aptamer is disposed on the planar surface of the fluidic device adjacent to the second subset of nucleic acid barcodes. In another aspect, the first aptamer and the second aptamer are disposed on an additional planar surface of the fluidic device, wherein the additional planar surface opposes of the planar surface. In a certain aspect, the first aptamer is disposed opposite of the first subset of nucleic acid barcodes, and wherein the second aptamer is disposed opposite of the second subset of nucleic acid barcodes.

Another embodiment of the present disclosure provides a fluidic device, comprising: one or more aptamers configured to bind to a cell or a component of the cell, wherein a first aptamer of the one or more aptamers is disposed on a first discrete area of the fluidic device, wherein a second aptamer of the one or more aptamers is disposed on a second discrete area of the fluidic device, and wherein the first aptamer is different from the second aptamer; and one or more nucleic acid barcodes disposed on a planar surface of the fluidic device, wherein the one or more nucleic acid barcodes comprise one or more capture sequences configured to hybridize to one or more cellular nucleic acids from the cell.

In a particular aspect, the first aptamer is configured to couple to the component of the cell, and wherein the component of the cell comprises a surface protein of the cell. In an additional aspect, the first aptamer is configured to couple to the component of the cell, and wherein the component of the cell comprises a protein secreted from first cell. In a further aspect, the protein secreted from the cell comprises a cytokine. In a certain aspect, the one or more nucleic acid barcodes further comprise one or more spatial barcode sequences associated with locations of the one or more nucleic acid barcodes on the planar surface of the fluidic device. In another aspect, the one or more nucleic acid barcodes further comprise one or more unique molecular identifier (UMI) sequences. In one aspect, the one or more cellular nucleic acids from the cell comprise mRNA molecules that encode surface proteins. In another aspect, the one or more cellular nucleic acids from the cell comprise guide RNA (gRNA) molecules that comprise barcode sequences associated with an identity of a surface protein of the cell. In a specific aspect, the one or more capture sequences comprise a poly T capture sequence configured to hybridize to polyA sequences on the one or more cellular nucleic acids from the cell.

In some aspects, the first discrete area and the second discrete area are located on the planar surface of the fluidic device. In additional aspects, the first aptamer is disposed on the planar surface of the fluidic device adjacent to a first subset of nucleic acid barcodes of the one or more nucleic acid barcodes, and wherein the second aptamer is disposed on the planar surface of the fluidic device adjacent to a second subset of nucleic acid barcodes of the one or more nucleic acid barcodes. In further aspects, the first discrete area and the second discrete area are located on an additional planar surface of the fluidic device, wherein the additional planar surface is located opposite of the planar surface. In certain aspects, the first aptamer is disposed opposite of a first subset of nucleic acid barcodes of the one or more nucleic acid barcodes, and wherein the second aptamer is disposed opposite of a second subset of nucleic acid barcodes of the one or more nucleic acid barcodes.

A further embodiment of the present disclosure provides a method, comprising: (a) introducing one or more cells into a fluidic device, wherein the fluidic device comprises one or more aptamers, wherein a first aptamer of the one or more aptamers couples to a first cell of the one or more cells or a component of the first cell, wherein a second aptamer of the one or more aptamers couples to a second cell of the one or more cells or a component of the second cell, wherein the first aptamer is different from the second aptamer; and (b) forming one or more chambers in the fluidic device, wherein a first chamber of the one or more chambers comprises (i) the first aptamer, (ii) the first cell, and (iii) a first subset of nucleic acid barcodes, wherein a second chamber of the one or more chambers comprises (i) the second aptamer, (ii) the second cell, and (iii) a second subset of nucleic acid barcodes, and wherein the first subset of nucleic acid barcodes and the second subset of nucleic acid barcodes are on a planar surface of the fluidic device.

In one aspect, the method further comprises releasing one or more cellular nucleic acids from the first cell and the second cell. In another aspect, the one or more cellular nucleic acids released from the first cell are captured by the first subset of nucleic acid barcodes, and wherein the one or more cellular nucleic acids released from the second cell are captured by the second subset of nucleic acid barcodes. In an additional aspect, the method further comprises (i) extending the first subset or the second subset of the nucleic acid barcodes to generate one or more extended nucleic acid barcodes or (ii) extending the one or more cellular nucleic acids released from the first cell or the second cell to generate one or more extended cellular nucleic acids. In a particular aspect, the method further comprises eluting the one or more extended cellular nucleic acid barcodes or the one or more extended cellular nucleic acids and sequencing the one or more extended nucleic acid barcodes or the one or more extended cellular nucleic acids. In a certain aspect, the method further comprises identifying the cell based on the sequencing. In a further aspect, the one or more cellular nucleic acids released from the first cell or the second cell comprise mRNA molecules that encode surface proteins. In an additional aspect, the one or more cellular nucleic acids released from the first cell or the second cell comprise guide RNA (gRNA) molecules that comprise barcode sequences associated with an identity of a surface protein of the first cell or the second cell. In another aspect, the method further comprises sequencing the one or more cellular nucleic acids from the first cell or the second cell or a derivative thereof. In a further aspect, the first aptamer binds to the component of the first cell, and wherein the component of the first cell comprises a surface protein of the first cell. In a select aspect, the first subset of nucleic acid barcodes comprise one or more first spatial barcode sequences associated with a location of the first subset of nucleic acid barcodes on the planar surface of the fluidic device, and wherein the second subset of nucleic acid barcodes comprise one or more second spatial barcode sequences associated with a location of the second subset of nucleic acid barcodes on the planar surface of the fluidic device.

An additional embodiment of the present application provides a fluidic device, comprising: one or more aptamers configured to bind to a cell or a component of the cell, wherein an aptamer of the one or more aptamers comprises an adapter sequence; and one or more nucleic acid barcodes, wherein a nucleic acid barcode of the one or more nucleic acid barcodes comprises a capture sequence that is configured to hybridize to the capturable sequence of the aptamer.

In some aspects, the aptamer is configured to bind to the component of the cell, and wherein the component of the cell comprises a surface protein of the cell. In further aspects, the aptamer is configured to bind to the component of the cell, and wherein the component of the cell comprises a protein secreted from the cell. In additional aspects, the protein secreted from the cell comprises a cytokine. In certain aspects, the fluidic device further comprises one or more chambers. In select aspects, a chamber of the one or more chambers comprises the aptamer, the cell, and the nucleic acid barcode. In particular aspects, an additional chamber of the one or more chambers comprises an additional aptamer of the one or more aptamers, an additional cell, and an additional nucleic acid barcode of the one or more nucleic acid barcodes. In some aspects, the nucleic acid barcode comprises a spatial barcode sequence unique to the chamber, and wherein the additional nucleic acid barcode comprises an additional spatial barcode sequence unique to the additional chamber. In another aspect, the one or more nucleic acid barcodes are located on a planar surface of the fluidic device. In one aspect, the one or more nucleic acid barcodes further comprise one or more spatial barcode sequences associated with a location of the one or more nucleic acid barcodes on the planar surface of the fluidic device. In a certain aspect, the one or more nucleic acid barcodes further comprise one or more unique molecular identifier (UMI) sequences. In another aspect of the method, the capturable sequence comprises a polyA sequence, and wherein the capture sequence comprises a poly A sequence. In a further aspect, the adapter is located on a 3′ end of the aptamer. In a certain aspect, the aptamer further comprises a primer sequence at a 5′ end of the aptamer. In a specific aspect, the one or more nucleic acid barcodes comprise: i) spatial barcode sequences associated with locations of the one or more nucleic acid barcodes on a surface of the fluidic device, ii) unique molecular identifier sequences, iii) oligonucleotide capture sequences, iv) chemically cleavable moieties, v) enzymatically cleavable moieties, vi) primer binding sites, or vii) a combination thereof. In another aspect, the one or more nucleic acid barcodes are configured to hybridize to one or more cellular nucleic acids from the cell. In a further aspect, the one or more cellular nucleic acids comprise mRNA molecules that encode surface proteins. In an additional aspect, the one or more cellular nucleic acids comprise guide RNA (gRNA) molecules, wherein the gRNA molecules comprise barcode sequences associated with an identity of a surface protein of the cell.

A further embodiment of the present disclosure provides a method, comprising: (a) combining one or more aptamers with a cell, wherein an aptamer of the one or more aptamers binds to the cell or a component of the cell, and wherein the aptamer comprises a capturable sequence, and (b) inputting the cell into a fluidic device, wherein one or more nucleic acid barcodes are coupled to a surface of the fluidic device, and wherein a nucleic acid barcode of the one or more nucleic acid barcodes is configured to hybridize to the adapter sequence of the aptamer.

In one aspect, a subset of the aptamers are not bound to the cell, and wherein the method further comprises removing the subset of the aptamers from the fluidic device. In another aspect, a subset of the aptamers are not bound to the cell, and wherein the method further comprises separating the subset of the aptamers from the cell. In a particular aspect, the method further comprises forming one or more chambers in the fluidic device, wherein a chamber of the one or more chambers comprises (i) the aptamer, (ii) the cell, and (iii) the nucleic acid barcode. In certain aspects, the aptamer binds to the component of the cell, and wherein the component of the cell comprises a surface protein of the cell. In further aspects, the method further comprises dissociating the aptamer from the cell. In a specific aspect, the dissociating comprises: i) lysing the cell, ii) reducing an ionic strength of a solution surrounding the cell, iii) increasing or reducing a pH of the solution surrounding the cell, iv) inputting a chaotropic agent into the fluidic device, v) inputting an organic solvent into the fluidic device, vi) increasing a temperature of the fluidic device, or vii) a combination thereof.

In another aspect, the method further comprises hybridizing the capturable sequence of the aptamer to a capture sequence of the nucleic acid barcode. In one such aspect, the method further comprises extending the nucleic acid barcode using at least a portion of the capturable sequence as a template to generate an extended nucleic acid barcode, extending the aptamer using at least a portion of the nucleic acid barcode as a template to generate an extended aptamer, or a combination thereof. In certain aspects, the method further comprises eluting the extended nucleic acid barcode or the extended aptamer from the fluidic device and sequencing the extended nucleic acid barcode or the extended aptamer. In particular aspects, the method further comprises identifying the cell based on the sequencing.

In some aspects, the method further comprises releasing one or more nucleic acids from the cell. In further aspects, the one or more nucleic acids released from the cell are captured by an additional nucleic acid barcode of the one or more nucleic acid barcodes. In particular aspects, the one or more nucleic acids released from the cell comprise mRNA molecules that encode surface proteins. In certain aspects, the one or more nucleic acids released from the cell comprise guide RNA (gRNA) molecule, wherein the gRNA molecule comprises a barcode sequence associated with an identity of a surface protein expressed by the cell. In some aspects, the one or more nucleic acid barcodes comprise one or more spatial barcode sequences associated with a location of the one or more nucleic acid barcodes on the surface of the fluidic device.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is a cross-sectional side view of a portion of a channel disposed in a fluidic device, according to some embodiments.

FIG. 2A is an illustration of a portion of a system as provided herein including an energy source for forming a polymer matrix being around a biological component, according to some embodiments.

FIG. 2B is an illustration of a polymer matrix being formed around another biological component in a portion of a system as provided herein, according to some embodiments.

FIG. 2C is an illustration of a top view of a polymer matrix formed around a biological component in a system as provided herein, according to some embodiments.

FIG. 3A is an illustration of a top view of the bottom layer of a flow cell.

FIG. 3B is an illustration of a top view of the spacer layer with a cut-out region suitable for use as part of the flow cell.

FIG. 3C is an illustration of a top view of the top layer of the flow cell where the top layer has an inlet and outlet opening.

FIG. 3D is an illustration of a cross-sectional side view of a top layer and a bottom layer sandwiching a spacer layer to form multiple channels of a flow cell.

FIG. 4A is an illustration of a system with a flow cell and an imaging apparatus that are capable of implementing methods of the present disclosure.

FIG. 4B is an illustration of a flow cell, including a blown-up view of a portion of a channel containing cells disposed in hydrogel chambers.

FIG. 5 is a schematic of a computer system 1501 that is configured to perform methods described herein.

FIG. 6 is a schematic of a method for identifying cell-binding aptamers and for separating the cell-binding aptamers from aptamers that do not bind cells.

FIGS. 7A-7B are schematics of methods for identifying aptamer binding targets. FIG. 7A depicts the method in a fluidic device in which aptamers and nucleic acid barcodes are coupled to separate surfaces of the fluidic device. FIG. 7B depicts the method in a fluidic device in which aptamers and nucleic acid barcodes are coupled to a common surface in the fluidic device.

FIGS. 8A-8B are schematics of methods for aptamer-based cell surfaceome analysis.

FIG. 8A depicts a method in which a cell is combined with aptamers before being input into a fluidic device. FIG. 8B depicts a method in which a cell is combined with aptamers after being input into a fluidic device.

FIG. 9 depicts a method for aptamer-based cell surfaceome analysis in which a cell is combined with aptamers prior to being partitioned within a droplet.

FIG. 10 illustrates a method for cell secretome analysis in which proteins secreted by the cell are captured by aptamers affixed to a surface of a fluidic device.

### DETAILED DESCRIPTION

The present disclosure provides methods for characterizing cells with aptamers. The disclosed methods expand upon the capabilities of existing cell characterization methods such as transcriptome profiling, which often returns large amounts of information unrelated to cell type, function, and state. On average, there are 500,000 mRNA molecules representing transcripts from 10,000-20,000 expressed genes, with varying levels of expression for each gene. In single-cell transcriptome analyses, approximately 10,000 reads are typically captured from each cell, corresponding to a detection sensitivity of around only 2%. Furthermore, the majority of these reads typically correspond to the housekeeping functions of a cell, which reduces the abundance of genes that define a cell's character. Housekeeping genes are expressed in thousands of copies, while most genes involved in regulatory functions and encoding for surfaceome and secretome are expressed in fewer than 100 copies per cell. Therefore, single-cell gene expression profiling often disproportionately observes genes that are highly expressed and misses transcripts related to cell behavior, identity, health, and function.

Comprehensive measurement of surface proteins may provide a more accurate characterization of a cell for several reasons. First, molecular transport through proteins and surface interactions for cell-cell communication are generally more cell-specific than transcriptomics. Second, the number of surface proteins involved in “housekeeping” functions is significantly lower than that of internal proteins. Third, the number of proteins on the surface of a cell is often greater than the number of transcripts inside of a cell by more than an order of magnitude. Fourth, in humans, the diversity of surface proteins is limited to approximately 1,700, which is about an order of magnitude less diverse than transcripts. Fifth, surface protein profiling can identify protein post-translational modifications (e.g., glycosylation) and conformation states that are otherwise undiscernible through transcriptomic analysis. Additionally, surface measurements are potentially less disruptive to the cell, as surfaceome profiling can leave a cell intact while transcriptome profiling typically requires cell lysis. Surfaceome signatures may thus provide a more accurate means for characterizing cell type and cell state (e.g., activated vs. resting) than transcriptomic analysis.

The present disclosure provides methods for generating and using aptamer libraries for cell surfaceome analysis. Many aptamer-based methods generate bulk data on collections of cells, and are therefore incapable of correlating individual cellular characteristics with aptamer binding data. The presently disclosed methods address this limitation by enabling real-time aptamer profiling that can be performed in tandem with other cellular analysis methods. The methods thus enable cell surfaceomic profiling, determined using aptamer libraries, to be performed on individual cells in tandem with measurements of additional cellular characteristics such as transcriptome, secretome, genetic modification, behavior, and viability. The methods enable a plurality of analyses to be performed and linked to individual cells on a sample size of thousands to millions of cells within a single individual fluidic device.

The disclosed aptamer-based approaches can provide several advantages over conventional methods for profiling cell surface proteomes. First, the disclosed aptamers can be readily multiplexed at high dimensionality with minimal spectral overlap, and can thereby enable simultaneous interrogation of tens to thousands of surface proteins to capture combinatorial activation signatures that are not accessible using limited-plex antibody panels. Second, owing to their small size, the disclosed aptamers can be selected to bind specific conformations or functional states of proteins, including ligand-bound, glycosylation, or activated receptor states, allowing discrimination between inactive and signaling-competent forms of the same protein. Third, the disclosed aptamers can be quantitatively measured through downstream nucleic acid readouts such as sequencing and qPCR, providing a broader dynamic range and improved sensitivity compared to fluorescence-based detection methods, rendering subtle changes in surfaceome detectable. Fourth, the disclosed aptamers can have small hydrodynamic volumes and conformational flexibility relative to detection proteins such as antibodies, and may thereby pack access confined and restricted epitopes along cell surfaces that may be otherwise inaccessible with other binding agents. In combination, these features can enable comprehensive, sensitive surface proteome profiling to characterize cell type, state, activity, and behavior.

As used herein, the term “aptamer” can refer to a nucleic acid molecule that has a binding affinity for a target molecule such as a protein. In various embodiments, the nucleic acid molecule can have a range of about 20 to about 100 nucleotides. Aptamers typically adopt secondary or tertiary structures that impart van der Waals surface complementarity for specific epitopes on target molecules. Aptamers can include DNA, RNA, naturally occurring non-canonical nucleic acids, non-naturally occurring nucleic acids, as well as combinations thereof. Many aptamers are single stranded nucleic acid molecules that partially self-hybridize to form secondary structures such as hairpin loops, bulge loops, base-pair stacks, and pseudoknots. However, the aptamers disclosed herein can also be double-stranded, triple stranded, G-quadruplexed, and the like.

As used herein, the term “aptamer binding portion” can refer to a portion of a nucleic acid molecule that has a binding affinity for a target molecule or contributes to secondary structure responsible for target molecule binding. Aptamer binding portions can be distinguished from primers, barcodes, and other nucleic acids that may flank an aptamer binding portion but do not contribute to its aptamer activity. As an example, an aptamer disclosed herein can include an aptamer binding portion flanked by identical primer sequences that do not affect the binding affinity of the aptamer for its epitope.

As used herein, the term “surfaceome” can refer to the collections of biomolecules on the surface of a cell and biomolecules exposed to the extracellular space outside of the cell. Cell surfaceomes are typically complex mixtures of biomolecules such as proteins, carbohydrates, peptides, glycopeptides, lipids, glycans and, in certain cases, metabolites. A biomolecule that is a part of a cell's surfaceome may be anchored to a cell surface membrane, a cell wall, to a biomolecule that is anchored to a cell surface membrane, or to a biomolecule that is anchored to a cell wall.

As used herein, the term “surface proteome” can refer to the collections of proteins on the surface of a cell and proteins exposed to the extracellular space outside of the cell.

Identify Aptamers that Bind to Cells

The present disclosure provides methods for identifying aptamers that couple to cells. This process may involve combining and then incubating a library of aptamers with one or more cells so that a first subset of the aptamers couple to the cell and a second subset of the aptamers do not couple to the one or more cells, and then separating the one or more cells and the first subset of aptamers coupled to the one or more cells from the second subset of aptamers that are not coupled to the one or more cells. Multiple sequential selection rounds can be performed in this manner, wherein the first subset of aptamers can be recombined with additional cells and aptamers that couple to the additional cells can be collected for further selection steps or for use in an omics assay. In between aptamer selection steps, an aptamer library or a selected subset of an aptamer library may be randomly evolved, for example through error prone polymerase chain reaction (PCR), site-directed mutagenesis, random recombination; rationally evolved, for example with a nucleic acid secondary structure prediction computational program and de novo nucleic acid synthesis; or a combination of random and rational library evolution steps. Synthesized aptamers may be made to be single strand using conventional techniques such as gel electrophoresis, a chaotropic agent, asymmetric amplification, strand-targeted pull down (e.g., in which a biotinylated strand is separated from a non-biotinylated strand during pull down), and the like. An aptamer library or a selected subset of an aptamer library can also be subjected to one or more negative or counter-selection steps, for example to remove aptamers that couple to an undesired antigen, to remove aptamers that couple to other aptamers, or to remove aptamers that couple to an undesired cell type. For example, an aptamer library can be subjected to positive selection steps against a first type of cell and negative selection steps against a second type of cell to select aptamers that couple to the first type of cell and do not couple to the second type of cell.

In one aspect, the present disclosure provides a method for identifying cell-binding aptamers that includes: combining aptamers with cells in a container, wherein each aptamer of the aptamers includes a first primer, an aptamer binding portion, and a second primer, wherein the first primer and the second primer are both coupled to the aptamer binding portion; wherein a first subset of the aptamers couple to the cells and a second subset of the aptamers do not couple to the cells; separating the second subset of the aptamers from the container; dissociating the first subset of aptamers from the cells, and sequencing the first subset of aptamers, thereby identifying the cell-binding aptamers. The first subset of aptamers may be amplified and/or evolved and then recombined with additional cells for further selection. The method may also be used for negative selection, wherein the second subset of cells are collected and sequenced. The method may be repeated one or more times with the same types of cells or with different types of cells. In various embodiments, the first primer and the second primer can have the same sequence or alternatively can be different. In some instances, the first primer can have about 90% or more of the same base pair sequence of the second primer (or vice versa).

The first and second primers may enable facile collection and amplification of the first subset of the aptamers. For example, following dissociation of the first subset of aptamers from the cells, the first subset of aptamers can be pulled down with a nucleic acid that couples to the first or second primer, and then amplified and/or indexed using the first and second primer sequences. In various embodiments, the nucleic acids can be coupled to a surface (e.g., fluidic device or bead). To facilitate exponential amplification and sequencing, the first and second primer sequences can be coupled to opposing ends of the aptamer binding portions of the aptamers.

The one or more cells used for aptamer identification can include a single cell type or multiple types of cells. The cells can be derived from a single organism, such as a human. The cells can include multiple cell types, for example, to select an aptamer library that generically couples to cells from a particular organism or tissue. The cells can, alternatively, include a single type or a collection of closely related cell types. The cells can be related by type, lineage, differentiation state, disease state, tissue, or organism. An exemplary method, for example, can utilize one to ten types of human cells. In certain cases, the cells include one to ten types of human cells. For example, the cell can include one to ten types of human immune cells. In various embodiments, the types of cells can include T-cells, CAR-T cells, muscle cells, cancer cells, cardiomyoctes, liver cells, fibroblasts, or neurons.

The aptamers can include an aptamer library with about 10¹¹ to about 10¹⁶ or about 10¹² to about 10¹⁴ unique aptamers. In such cases, only a subset of the aptamers may have binding affinities for the cells. The method may also be performed on a selected subset of an aptamer library. For example, the method may first be performed on a library with about 10¹¹ to about 10¹⁶ unique aptamers to generate a first subset of aptamers, and then repeated on an amplified and optionally evolved (e.g., through error-prone PCR) population of aptamers generated from the first subset of aptamers. In such cases, all of the aptamers or the majority of the aptamers may have binding affinities for the cells. In various embodiments, each aptamer of the aptamers library can have the same number of base pairs (e.g., 20 to 100 nucleotides) and be generated in a random manner to create numerous unique sequences of nucleotides.

Numerous separation methods may be used to remove the second subset of the aptamers from the container that holds the cells. In some cases, the removing includes one or more wash steps, wherein the first subset of the aptamers remain coupled to the cell during the one or more wash steps. For example, a plurality of buffer addition and removal steps can be performed with filter tipped pipettes with pores that are sufficiently large to collect solubilized aptamers but prohibitively small for the cells to pass through. Alternatively, the method may include sedimenting the cells to a bottom surface of the container, and only removing a top portion of fluid in the container during a wash step. In some cases, the removing includes centrifuging the container (or an additional container into which the cells are transferred), thereby generating a supernatant that includes the second subset of the aptamers and a pellet that includes the cell and the first subset of the aptamers coupled to the cell, and separating the supernatant from the pellet. Alternatively, the separating can include coupling a magnetic particle to the cell, wherein the cell is coupled to the first subset of the aptamers, and magnetically separating the magnetic particle coupled to the cell from the second subset of the aptamers. Before combining the cell and the aptamer together, the magnetic particle can be derivatized with biotin, streptavidin, or an antibody that can then couple to the cell. The separating can also include filtering a liquid in the container, thereby generating a filtrate that includes the second subset of the aptamers and a residue that includes the cell and the first subset of the aptamers coupled to the cell.

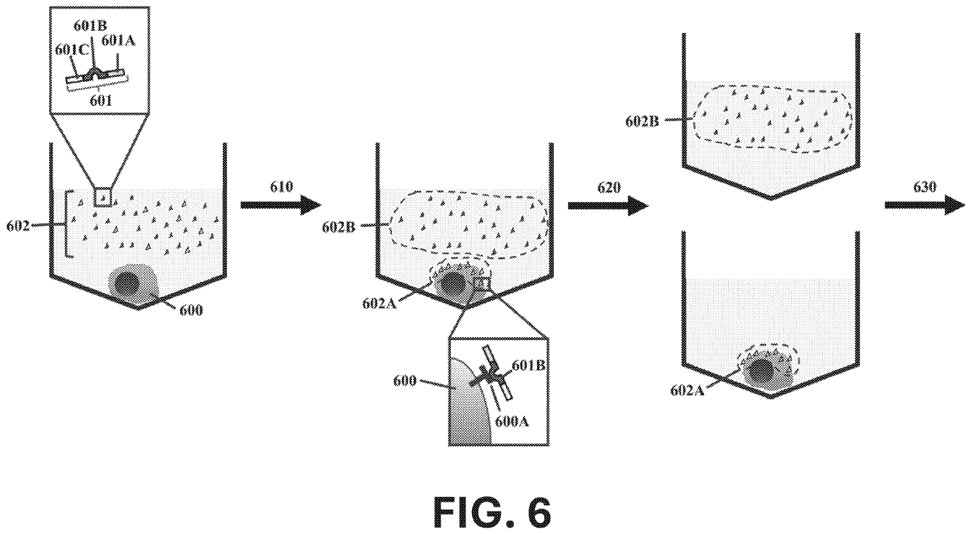

An example of a method for identifying aptamers that bind to cells is depicted in FIG. 6. In this example, an aptamer library 602 is combined with a cell 600. The aptamer library 602 may be a naïve aptamer library, such as a randomly generated aptamer library, of which only a subset of aptamers bind to the cell 600. For example, the aptamer library may include from 10¹¹ to 10¹⁶ randomly generated aptamers, of which only a subset of the randomly generated aptamers couple to the cell 600. Each aptamer 601 includes an aptamer binding portion 601B that can have an affinity for a surface protein on the cell 600. Alternatively, each aptamer 601 can include a randomly generated or semi-randomly generated sequence that may bind to a surface protein on the cell 600. Each aptamer 601 may include a first primer 601A, a second primer 601C, or a combination of a first primer 601A and a second primer 601C. The primer or primers (601A & 601C) may facilitate aptamer capture and amplification. For example, the first primer 601A and the second primer 601C may include primer binding sequences, ligation handles, capture sequences (e.g., polyA tails), chemical modifications (e.g., biotin or a strep-tag II for binding to streptavidin), or sequencing adapters. The aptamers 601 may also include a barcode sequence that identifies the aptamer binding portion 601B, a sequence that normalizes sequencing counts (e.g., a unique molecular identifier sequence), an indexing sequence associated with an assay or cell, an index or adapter for next generation sequencing. As depicted in FIG. 6, first primer 601A and second primer 601C may be coupled to opposite ends of the aptamer binding portion 601B of the aptamer 601. For example, the first primer 601A may be coupled at a 3′ end of the aptamer binding portion 601B and the second primer 601C may be coupled at a 5′ end of the aptamer binding portion 601B.

Referring back to FIG. 6, the aptamer library 602 is incubated with the cell 600, during which time a first subset of aptamers of the aptamer library 602A is bound to the cell 600 while a second subset of aptamers of the aptamer library 602B remained unbound from the cell 600. Aptamer binding may be reflective of a competitive binding process in which multiple aptamers compete for limited space or protein epitopes on the surface of the cell 600. Under such conditions, the first subset of aptamers 602A may change with time, as fast binding high mobility aptamers are replaced by higher affinity, slower binding aptamers on the surface of the cell. The incubation time, conditions, and cell-to-aptamer ratio may be adjusted to favor high mobility or high affinity aptamer binding. The method of FIG. 6 may also be performed multiple times to collect early- and late-binding aptamers.

In a subsequent step 620, the first subset of aptamers 602A (bound to the cell 600) may be separated from the second subset of aptamers 602B (not bound to the cell 600). Prior to the separation, the first subset of aptamers 602A and the second subset of aptamers 602A may be disposed in a single container. The separation 620 may partition the first subset of aptamers 602B and the second subset of aptamers 602B into separate volumes of liquid. For example, the second subset of aptamers 602B may be separated from the cell through multiple wash steps that serially dilute the second subset of aptamers 602B without removing the cell 600 or the first subset of the aptamers 602A coupled to the cell 600 from the container. Similarly, the second subset of aptamers 602B may be separated from the cell 600 and from the first subset of aptamers coupled to the cell 602A by dialysis and/or filtration. The separation 620 can also include centrifugation, which may generate a supernatant containing the second subset of aptamers 602B and a pellet containing the cell 600 and the first subset of aptamers 602A coupled to the cell 600. Following centrifugation, the supernatant can be removed (e.g., pipetted) and the pellet can be resuspended to generate a solution containing the cell 600 and the first subset of aptamers 602A. An example of a method for centrifuging cell-aptamer complexes is provided in Safeh et al., Nat. Protoc., 2010; 5:1169, which is incorporated by reference for its disclosure directed to aptamer separation through centrifugation. The first and second subset of aptamers (602A, 602B) may also be separated with a particle. The particle may include an antibody or other binding agent that targets a surface protein on the cell 600. After the antibody or other binding agent on the particle couples to the cell 600, the particle with the bound cell may be magnetically separated from the second subset of aptamers 602B. For example, the particle may be magnetically affixed to a surface within a container and subjected to wash steps that remove the second subset of aptamers 602B but do not dissociate the cell 600 from the particle or the first subset of aptamers 602A from the cell.

Following the separating 620, the first subset of aptamers 602A can be dissociated from the cell, optionally amplified, and sequenced in step 630. The first subset of aptamers 602A can be utilized to generate a new aptamer library, for example through error-prone replication as disclosed in Wachowius et al., Chem Systems Chem, 2023, 5, e202300006, which is incorporated herein by reference for its disclosure on error-prone replication for aptamer library generation. Cell-binding aptamers may then be selected from the new aptamer library according to the method outlined in FIG. 6.

### Identify Aptamer Binding Targets

Further disclosed herein are methods for identifying the binding targets of one or more aptamers. The aptamers may be a subset of aptamers from a larger aptamer library that are known to bind to a certain type of cell, such as the first subset of aptamers identified during a method for identifying cell-binding aptamers as outlined in FIG. 6. The method may identify aptamers that bind to a single target molecule, or, more specifically, to a single aptamer epitope on a single target molecule. Such aptamers may be particularly useful for surfaceomic (e.g., measurement of proteins or peptides at the surface of a cell or at least partially exposed to the extracellular space) analysis, as the presence of the aptamers on the surface of a cell can signify the presence of a particular protein on the surface of the cell.

In one aspect, a method for identifying aptamer binding targets includes inputting cells into a fluidic device, wherein the fluidic device comprises a first surface, a second surface, and an array of discrete spots on the first surface, wherein each site of the array comprises an aptamer configured to couple to a surface protein on a cell; coupling the surface protein on the cell to the aptamer for at least one site of the array; synthesizing chambers, wherein each chamber co-encloses the cell, the aptamer coupled to the surface protein on the cell, and a nucleic acid barcode; wherein each nucleic acid barcode is coupled to i) the first surface at each site of the array or ii) the second surface at a location of the fluidic device coincident with each site of the array, and wherein the nucleic acid barcodes include: i) spatial barcode sequences associated with locations of the nucleic acid barcodes in the fluidic device, and ii) capture sequences configured to hybridize to cellular nucleic acids from the cells; releasing the cellular nucleic acids from the cells; capturing the cellular nucleic acids on the nucleic acid barcodes, and i) extending the nucleic acid barcodes using at least a portion of the cellular nucleic acids as a template, and sequencing the extended nucleic acid barcodes; ii) extending the cellular nucleic acids using at least a portion of the nucleic acid barcodes as a template, and sequencing the extended nucleic acids; or iii) a combination thereof; and associating the aptamers with the surface proteins on the cells based on the sequencing. It should be noted that where each nucleic acid barcode is coupled to the second surface at a location of the fluidic device coincident with each site of the array, then each of the locations corresponds to an area on the second surface that is defined by a perpendicular projection from a peripheral portion of each discrete spot on the first surface.

Exemplary applications of the method for identifying aptamer binding targets utilize cells that encode a protein library, wherein each cell encodes a single protein from the protein library. Such a library may be comprised of proteins from a single organism, such as a human. The library may be expressed heterologously in an organism that does not natively express proteins from the protein library. For example, the expression library can include a yeast human proteome display library as disclosed in Bidlingmaier & Liu, Methods Mol Biol, 2015; 1319:203, which is herein incorporated by reference for its disclosure on human proteome libraries and yeast display.

While yeast cells are disclosed as an exemplary embodiment, other cell types such as bacteria may be utilized for identifying aptamer binding targets. A cell library may be generated using one or more gene editing, transduction, and/or transfection techniques to introduce nucleic acid constructs encoding proteins of interest into the cells. Nucleic acid constructs may then be integrated at the cleavage locations. The nucleic acids may be introduced into cells using viral vectors such as lentiviral, retroviral, or adenoviral vectors; using a transfection method such as electroporation, sonoporation, optoporation, lipid-mediated delivery, and/or nanoparticle-based delivery; or a combination thereof. Cells may be stably transfected, for example by cleaving genomic DNA and inserting nucleic acid constructs at the cleaved sites. Such gene editing methods may be performed using, for example, CRISPR/Cas systems, TALENs, zinc finger nucleases, restriction endonucleases, and/or argonaut proteins to cleave genomic DNA at targeted loci. Nucleic acid constructs may then be integrated into the cleaved genomic DNA through transposition, ligation, and/or recombination (e.g., non-homologous end joining (NHEJ), homology-directed repair (HDR)). Cells may also be transiently transfected to achieve short-term expression of introduced constructs without genomic integration. The introduced constructs may include promoters, regulatory elements, barcodes, and/or selection markers. The edited cells may express one non-native protein per cell. For example, in certain implementations, pooled approaches are employed such that a heterogeneous population of cells collectively encodes a diverse set of proteins, and the relative representation of each engineered cell type may be controlled by vector design, multiplicity of infection, or selection conditions. Such libraries may be further expanded, selected, or enriched to obtain desired expression characteristics, thereby enabling downstream screening, profiling, or functional characterization of protein expression in a high-throughput manner.

Alternatively, in addition to analyzing surface proteins on cells, proteins may be analyzed on the surfaces of viruses, on the surfaces of ribosomes, or as protein-mRNA complexes. In some implementations of the presently disclosed methods, a protein library is expressed in a phage display library, wherein proteins are expressed on the surfaces of viruses that contain genetic material encoding the proteins. Viruses that bind to an aptamer may be lysed to release their genetic material for capture on nucleic acid barcodes and sequencing as disclosed elsewhere herein. Examples of viruses consistent with phage display library construction include M13 bacteriophages, filamentous bacteriophages, Escherichia viruses, enterobacteria phages, as well as viruses disclosed in Pande et al., Biotechnology Advances, 2010; 28:849, which is herein incorporated by reference for its disclosure on phage display libraries and phage display library construction.

Proteins may also be provided within ribosome display libraries. Ribosome display libraries are comprised of ternary mRNA-ribosome-protein complexes in which a ribosome is simultaneously coupled to a protein and mRNA encoding the protein. Following protein capture, the ribosomes may be dissociated from the mRNA and proteins, freeing the mRNA for capture and analysis as disclosed elsewhere herein. Examples of ribosome display methods are provided in Lipovsek and Plucktun, J. Immunol. Meth., 2004; 290:51, which is herein incorporated for its disclosure on ribosome display libraries and ribosome display library construction.

Alternatively, proteins may be provided in mRNA display libraries, which include protein-mRNA complexes comprised of proteins coupled to the mRNA that encodes the proteins. When implemented in the presently disclosed methods, mRNA display libraries may be input into a fluidic device that includes surface-bound aptamers. After one or more protein-mRNA complexes bind to the surface-bound aptamers, unbound protein-mRNA complexes may be removed from the fluidic device, and mRNA can be released from aptamer-bound protein-mRNA complexes for analysis as disclosed elsewhere herein. Examples of mRNA display methods are disclosed in Lipovsek and Plücktun (2004), which is further incorporated herein for its disclosure on mRNA display libraries and mRNA display library construction.

The cellular nucleic acids may encode the surface proteins on the cell or include barcode sequences associated with identities of the surface proteins on the cell. As a first example, the cellular nucleic acids can include mRNA molecules that encode the surface proteins. Similarly, the cellular nucleic acids can include gDNA fragments that encode the surface proteins. Alternatively, the cellular nucleic acids can include gRNA molecules that include barcode sequences associated with an identity of the surface proteins or genetic edit.

The cellular nucleic acids can be released through lysis or by a similar method such as cell permeabilization. Once the cellular nucleic acids are released, they can be captured on the nucleic acid barcodes. The nucleic acid barcodes typically include oligonucleotide capture sequences that are configured to hybridize to at least a portion of the cellular nucleic acids. In an exemplary embodiment, the oligonucleotide capture sequences include poly T sequences that are configured to hybridize to poly A tails on the cellular nucleic acids. The nucleic acid barcodes can include spatial barcode sequences associated with locations of the nucleic acid barcodes on the surface of the fluidic device, such as a discrete spot within the fluidic device that includes a single type of aptamer. The nucleic acid barcodes can also optionally include unique molecular identifier sequences that may be used to normalize sequencing reads on amplified populations of extended nucleic acid barcodes or extended cellular nucleic acids. In particular embodiments, the nucleic acid barcodes include enzymatically or chemically cleavable moieties, such as restriction enzyme binding sites, that allow the nucleic acid barcodes to be cleaved from the first or second surface of the fluidic device and eluted from the fluidic device. The nucleic acid barcodes can also include primer binding sites, sequencing adapter sites, or other sequences that facilitate downstream amplification or analysis.

In many aspects of the method for identifying aptamer binding targets, the aptamers are coupled the first surface. The aptamers may be coupled to the first surface through a biomolecular or synthetic linker. In an exemplary embodiment, the aptamers are coupled to the first surface through a nucleic acid linker. The linker may be cleavable, thereby allowing the aptamers to be released from the first surface and eluted from the fluidic device for downstream analysis. The aptamers may optionally include spatial barcode sequences associated with locations of the aptamers in the fluidic device, unique molecular identifier sequences, barcode sequences associated with nucleic sequences of the aptamers, chemically cleavable moieties, enzymatically cleavable moieties, primer binding sites, or a combination thereof. One or more of the unique molecular identifier sequences, barcode sequences associated with nucleic sequences of the aptamers, chemically cleavable moieties, enzymatically cleavable moieties, primer binding sites, or the combination thereof may be disposed within a linker portion of the aptamer through which the aptamer is coupled to the first surface.

The aptamers may also be hybridized to a subset of the nucleic acid barcodes. In such cases, each aptamer may include a capturable sequence that is complementary to a spatial barcode sequence of a nucleic acid barcode within the fluidic device, and may optionally further include a barcode sequence associated with the sequence of the aptamer binding portion of the aptamer. The aptamer may thus migrate and hybridize to a nucleic acid barcode within a particular discrete spot in the fluidic device.

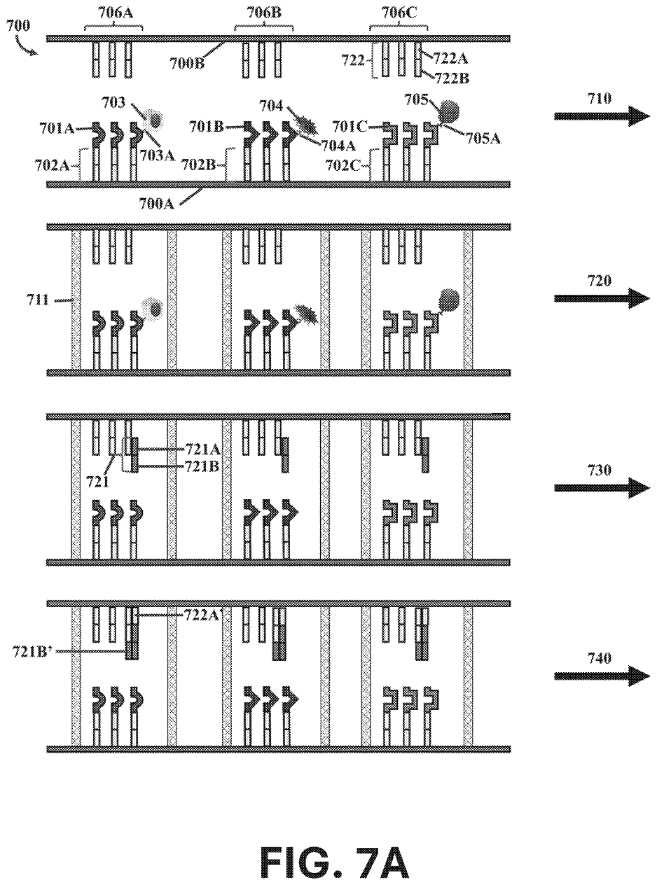

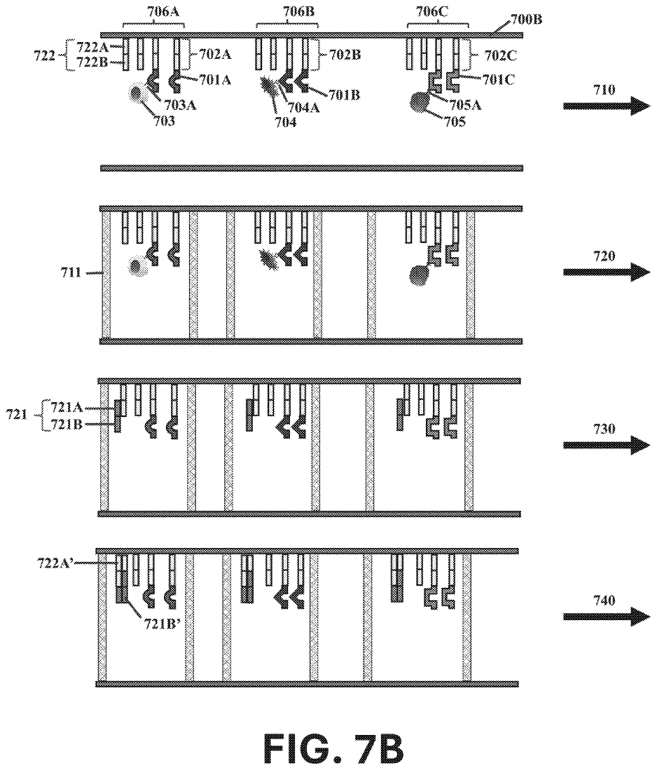

FIG. 7A illustrates an example of a method for identifying surface proteins targeted by one or more aptamers from an aptamer library. The aptamer library may be known to bind to surface proteins on a particular type of cell, but may have unknown surface protein targets on that cell. For example, the aptamer library may have been selected using the process illustrated in FIG. 6, and thus may be a subset of a larger aptamer library that is known to bind to surface proteins on a particular cell of interest, but for which the specific surface protein targets are not known. In FIG. 7A, multiple cells (703, 704, 705) are input into a fluidic device 700 that includes a library of aptamers (701A, 701B, 701C). The aptamers (701A, 701B, 701C) are coupled to a first surface of the fluidic device 700A through linkers (702A, 702B, 702C), and are arranged in locations on the first surface coincident with discrete spots (706A, 706B, 706C) along the second surface 700B, with each of the locations containing a single type of aptamer. In this example, each discrete spot (706A, 706B, 706C) also contains nucleic acid barcodes 722 that are coupled to a second surface 700B that faces corresponding locations on the first surface 700A. The nucleic acid barcodes 722 are at locations that are coincident with the aptamers (701A, 701B, 701C) on the first surface 700A. Each nucleic acid barcode 722 contains a spatial barcode sequence 722A associated with its location within the fluidic device 700 and a capture sequence 722B that is configured to hybridize to at least a portion of the nucleic acid from one or more of the cells. As each location, on the first surface coincident with corresponding discrete spot (706A, 706B, 706C), includes a single type of aptamer (701A, 701B, 701C), each spatial barcode sequence 722A can be uniquely associated with a single aptamer.

Each of the cells (703, 704, 705) expresses a single surface protein (703A, 704A, 705A) from a surface protein library. The surface proteins (703A, 704A, 705A) couple to the aptamers (701A, 701B, 701C), thereby localizing each cell (703, 704, 705) to a discrete spot (706A, 706B, 706C) along the first surface of the fluidic device 700A. FIG. 7A illustrates a first discrete spot 706A that is coincident with a location of a first aptamer 701A that couples to a first surface protein 703A expressed by a first cell 703, a second discrete spot 706B that is coincident with a location of a second aptamer 701B that couples to a second surface protein 704A expressed by a second cell 704, and a third discrete spot 706C that is coincident with a location of a third aptamer 701C that couples to a third surface protein 705A expressed by a third cell 705. However, the fluidic device 700 may include hundreds, thousands, tens of thousands, hundreds of thousands, or millions of discrete spots, which may each include a different aptamer. As non-limiting examples, the fluidic device can contain between about 10² and about 5×10², between about 10² and about 10³, between about 10² and about 5×10³, between about 10² and about 10⁴, between about 10² and about 5×10⁴, between about 10² and about 10⁵, between about 10² and about 5×10⁵, between about 10² and about 10⁶, between about 10² and about 5×10⁶, between about 5×10² and about 10³, between about 5×10² and about 5×10³, between about 5×10² and about 10⁴, between about 5×10² and about 5×10⁴, between about 5×10² and about 10⁵, between about 5×10² and about 5×10⁵, between about 5×10² and about 10⁶, between about 5×10² and about 5×10⁶, between about 10³ and about 5×10³, between about 10³ and about 10⁴, between about 10³ and about 5×10⁴, between about 10³ and about 10⁵, between about 10³ and about 5×10⁵, between about 10³ and about 10⁶, between about 10³ and about 5×10⁶, between about 5×10³ and about 10⁴, between about 5×10³ and about 5×10⁴, between about 5×10³ and about 10⁵, between about 5×10³ and about 5×10⁵, between about 5×10³ and about 10⁶, between about 5×10³ and about 5×10⁶, between about 10⁴ and about 5×10⁴, between about 10⁴ and about 10⁵, between about 10⁴ and about 5×10⁵, between about 10⁴ and about 10⁶, between about 10⁴ and about 5×10⁶, between about 5×10⁴ and about 10⁵, between about 5×10⁴ and about 5×10⁵, between about 5×10⁴ and about 10⁶, between about 5×10⁴ and about 5×10⁶, between about 10⁵ and about 5×10⁵, between about 10⁵ and about 10⁶, between about 10⁵ and about 5×10⁶, between about 5×10⁵ and about 10⁶, between about 5×10⁵ and about 5×10⁶, or between about 10⁶ and about 5×10⁶ discrete spots. In many aspects, each spot contains a single type of aptamer. For example, each aptamer of the single type of aptamer can be represented by a plurality of aptamers having the same sequence. It is worth noting that multiple cells may couple to aptamers within a discrete site. For example, in some cases, an aptamer may have binding affinity for two or more surface proteins, and may thus couple to two or more cells from a cell library. Similarly, an aptamer may have binding affinity for a surface protein natively expressed by cells from a cell library, in addition to surface proteins introduced into the cell library (for example through transfection or transduction). Such occurrences may be minimized by evolving an aptamer library to not bind to surface proteins of a particular cell type, as outlined for FIG. 6. Such occurrences may also identify multispecificity of a particular aptamer.

Polymeric chambers 711 can then be synthesized 710 to co-enclose the cells (703, 704, 705) with aptamers (701A, 701B, 701C) and nucleic acid barcodes 722. Each polymeric chamber 711 can enclose or be disposed within an individual discrete spot (706A, 706B, 706C) to enclose a single type of aptamer (701A, 701B, 701C) from the aptamer library. While FIG. 7A illustrates a single cell (703, 704, 705) within each chamber 711, a method may enclose multiple cells within a single chamber (e.g., when an aptamer has binding affinity to more than one cellular protein), or may enclose a single type of cell in multiple chambers (e.g., when multiple aptamers have binding affinity to a single surface protein). It is worthwhile to note that the chamber 711 is illustrated as a cross-section view in FIG. 7A. Chamber 711 can include a cylindrically shaped polymeric wall for containing a cell in an interior portion of the chamber. Chamber 711 can be bounded at the top by second surface 700B and at the bottom by first surface 700A. The polymeric wall can have a porosity configured to retain the cell within the chamber and allow reagent chemicals sufficiently smaller than the cell to permeate through the wall.

After the cells are enclosed within chambers 711, cellular nucleic acids 721 can be released 720 from the cells (703, 704, 705). In various embodiments, before the releasing step 720, various phenotypic measurements may be performed such as morphology, actuation, suppression, and/or cytokine secretions. The cellular nucleic acids 721 then diffuse to the nucleic acid barcodes 722, where nucleic acid barcode capture sequences 722B hybridize to complementary sequences on the nucleic acids 721A. The chambers 711 may trap the cellular nucleic acids 721, thereby preventing them from diffusing to barcodes 722 outside of their respective chambers. Alternatively, chambers 711 may significantly reduce the diffusion rate of cellular nucleic acids 721 to an area outside of the chamber 711, thereby reducing the likelihood of cellular nucleic acids 721 diffusing outside on one chamber and then flowing into another adjacent chamber. The chambers 711 may optionally be degraded after the nucleic acids 721 have hybridized to the barcodes 722.

In a subsequent extension step 730, the cellular nucleic acids 721 can be extended using the spatial barcode sequences 722A of the nucleic acid barcodes as templates and/or the nucleic acid barcodes can be extended using the protein encoding sequence 721B of the cellular nucleic acids as templates, thereby generating extended cellular nucleic acids that include complements of the spatial barcode sequences 722A′ and/or extended nucleic acid barcodes that include complements of protein encoding sequences 721B′. The nucleic acid barcodes may optionally contain further sequences which may serve as templates for extension of the cellular nucleic acids 721, such as unique molecular identifier sequences or primer binding sites.

The extended nucleic acid barcodes, the extended nucleic acids, or the combination thereof can then be sequenced in step 740. This step may include eluting the extended nucleic acid barcodes and/or the extended nucleic acids from the fluidic device, optionally amplifying and tagging the extended nucleic acid barcodes and/or extended nucleic acids (e.g., with sequencing adaptors), and sequencing the extended nucleic acid barcodes and/or extended nucleic acids on a next generation sequencing platform. As each discrete spot (706A, 706B, 706C) includes a single type of aptamer (701A, 701B, 701C), the spatial barcode sequences 722A on the extended nucleic acid barcodes or the complements of the spatial barcode sequences 722A′ on the extended nucleic acids can be associated with a single aptamer in the fluidic device 700. Similarly, the nucleic acid protein encoding sequence 721B or the complements of the protein encoding sequences 721B′ may encode or may be associated with a single surface protein (703A, 704A, 705A). For example, the cellular nucleic acids 721 can include mRNA that encodes the surface proteins (703A, 704A, 705A). Accordingly, sequencing the extended nucleic acid barcodes or extended nucleic acids may associate an individual aptamer with a surface protein to which the aptamer binds.

FIG. 7B depicts an alternative to the method of FIG. 7A in which the aptamers (701A, 702A, 703A) are coupled to the same surface 700B as the nucleic acid barcodes 722. In such a method, the aptamers (701A, 701B, 701C) may be coupled to the surface 700B through linkers (702A, 702B, 702C) as depicted in FIG. 7B. Alternatively, the aptamers (701A, 701B, 701C) may be hybridized to the nucleic acid barcodes 722. For example, the aptamers (701A, 701B, 701C) may be coupled to nucleic acids that include complements of the spatial barcode sequences 722A on the nucleic acid barcodes 722, such that the aptamers hybridize to nucleic acid barcodes in a single discrete spot (706A, 706B, 706C) in the fluidic device 700. It should be noted that although the aptamers (701A, 702A, 703A) and the nucleic acid barcodes 722 are coupled to the top surface 700B, as illustrated in FIG. 7B, it is also possible aptamers (701A, 702A, 703A) and the nucleic acid barcodes 722 are coupled to the bottom surface 700A.

### Surfaceomic Analysis

Further aspects of the present disclosure provide methods for analyzing a cell surface proteome with an aptamer library. The aptamer library may be generated and/or selected with one or more disclosed methods, such as the selection method depicted in FIG. 6 and the target-identification method depicted in FIG. 7A or 7B. Each aptamer in the aptamer library may target a different protein or protein epitope, such that a surface proteomic profile can be determined by identifying the aptamers that couple to the surface of the cell. The surface proteomic profile may then be used to identify the cell or a characteristic or state of the cell.

In some aspects, surface proteomic profile may be used to determine cell identities within heterogeneous samples. As outlined in Example 2, different cell types may be distinguished based on aptamer binding profiles. Such methods may be used to distinguish cell types of interest from complex biological samples such as dissociated tissue, blood fractions, and tumor sections. For example, in peripheral blood mononuclear cell (PBMC) samples, multiplex aptamer profiles may be used to resolve immune cell classes such as T cells, B cells, and natural killer cells, as well as finer subpopulations such T cell subsets and memory phenotypes (e.g., Th1, Th2, Th17, Treg, Tc1, Tc2, Tc9, Tc17, etc.). Such analysis can include coordinated detection of lineage-defining surface proteins (e.g., CD4 and CD8 for T cells and CD56 for natural killer cells), but also may include surface markers and surface marker ratios that evidence further characteristics such as cell state, activation status, and health status. As a further example, the disclosed methods may characterize cells from dissociated tissue samples such as tumor biopsies. Such analyses may identify cell types present within a dissociated tissue sample or a portion of a dissociated tissue sample, such as epithelial, stromal, and immune components of a dissociated tumor. More particularly, surface proteomic profile may be used to identify disease progress state. In such applications, aptamer binding signatures may distinguish normal cells from diseased cells. The diseased cells may be cancer cells, virally or bacterially infected cells, cells with genetic disorders, autoimmune-related cells, degenerative disease (e.g., steatosis fat-laden liver cells or muscular dystrophy cells with structural protein defects), cells with environmental or toxic damage, inflammation, as well as combinations thereof. For example, cancer cells may be characterized based on altered expression of oncogenic receptors, adhesion markers, immune evasion markers, surface protein glycosylation, and receptor expression and state. Surface proteomic profile is used to identify cells undergoing phenotypic transitions, such as epithelial-to-mesenchymal transition (EMT) or mesenchymal-to-epithelial transition (MET) exhibited by some forms of cancer cells. As an example, aptamer binding patterns may capture changes in adhesion molecule expression, cytoskeletal-associated surface proteins, and motility-related receptor expression that can be captured in aptamer-binding profiles.

Moreover, aptamer binding profiles may return information cell state. While some cell states can be evidenced by a small number of surface markers, many shifts in cell state are evidenced by subtle shifts in surface protein expression, conformation, and spatial organization. For example, exhausted T cells can share surface proteomic features with recently activated effector T cells, and furthermore can differentiate into biologically distinct sub-states, including progenitor-exhausted and terminally exhausted populations. Similarly, quiescent and senescent cells can exhibit similar surface proteomic profiles while nonetheless residing at different stages of their life cycles. The presently disclosed methods can address this type of challenge by providing deep proteomic profiling on single cells to distinguish cell populations with similar proteomic profiles.

In one aspect, a method for cellular analysis includes combining aptamers with a cell, wherein a first subset of the aptamers couple to the cell and a second subset of the aptamers do not couple to the cell; separating the second subset of the aptamers from the cell; inputting the cell into a fluidic device, wherein the fluidic device comprises nucleic acid barcodes; hybridizing capturable sequences of the first subset of the aptamers to the nucleic acid barcodes; extending the nucleic acid barcodes to generate extended nucleic acid barcodes or extending the first subset of the aptamers to generate extended aptamers; eluting the extended nucleic acid barcodes or the extended aptamers from the fluidic device; and sequencing the extended nucleic acid barcodes or the extended aptamers.

The first subset of aptamers may be associated with identities of one or more surface proteins on the cell. In particular, each aptamer may be associated with a single surface protein or epitope of a surface protein, so that identifying the sequences of the first subset of aptamers can be used to determine the identities of proteins on the surface of the cell. In some cases, the cell may be identified based on the first subset of aptamers that bind to the cell, or based on a surface proteome of the cell determined based on the first subset of aptamers.

The second subset of the aptamers can separated from the cell within the fluidic device or in a container that is separate from the fluidic device. In some cases, the combining and separating are prior to the inputting. In other cases, the combining and separating are subsequent to the inputting, the aptamers are input into the fluidic device, and the separating comprises removing the second subset of the aptamers from the fluidic device. Alternatively, the inputting can be subsequent to the combining and prior to the separating. For example, the aptamers can be input into the fluidic device along with the cell, and the separating can include removing the second subset of the aptamers from the fluidic device.

In many cases, the separating is performed before the cell is input into the fluidic device. In such cases, the cell can be combined with the aptamers in a container, and the separating can include removing the second subset of the aptamers from the container. For example, the separating can include centrifuging a composition comprising the cell and the aptamers, thereby generating a supernatant comprising the second subset of the aptamers and a pellet comprising the cell and the first subset of the aptamers coupled to the cell, and separating the supernatant from the pellet. Alternatively, the separating can include coupling a magnetic particle to the cell, wherein the cell is coupled to the first subset of the aptamers, and magnetically separating the magnetic particle coupled to the cell from the second subset of the aptamers. The separating can also include filtering a composition comprising the cell and the aptamers, thereby generating a filtrate comprising the second subset of the aptamers and a residue comprising the cell and the first subset of the aptamers coupled to the cell.

The separating can also be performed after the cell is input into the fluidic device. Such a method can include removing the second subset of the aptamers from the fluidic device in one or more wash steps, wherein the first subset of the aptamers remain coupled to the cell during the one or more wash steps. For example, the method can include synthesizing one or more chambers that co-enclose the cell, the first subset of aptamers coupled to the cell, and nucleic acid barcodes coupled to a surface of the fluidic device, and performing one or more wash steps to remove the second subset of the aptamers from the fluidic device while the cell is trapped within the one or more chambers and the first subset of the aptamers are coupled to the cell. The one or more chambers can have permeable polymeric walls configured to allow aptamers to pass through the polymeric walls and not allow cells to pass through the polymeric walls.

Alternatively, the method can include synthesizing one or more chambers that co-enclose the cell and nucleic acid barcodes coupled to a surface of the fluidic device, combining the aptamers with the cell by inputting the aptamers into the fluidic device, wherein a first subset of the aptamers couple to the cell and the second subset of the aptamers do not couple to the cell, and removing the second subset of the aptamers from the fluidic device in one or more wash steps. In such cases, the cell may be incubated within the fluidic device, and optionally within the one or more chambers within the fluidic device, before the cell is combined with the aptamers, thereby allowing the cell to be incubated within the fluidic device prior to surface proteome characterization with the aptamers.

In general, the separating removes the second subset of the aptamers from the proximity of the cell. For example, the separating may create a distance of at least about 1 centimeter between the cell and the second subset of the aptamers. In many cases, the separating moves the second subset of the aptamers and the cell (coupled to the first subset of the aptamers) into separate containers. For example, the second subset of the aptamers can be drawn into a syringe through a filter-tipped needle that prevents the cell and the first subset of the aptamers coupled to the cell from being drawn into the syringe. The separation may have an efficiency of from about 50% and about 100%, from about 75% and about 100%, from about 90% to 100%, from about 95% to about 100%, from about 98% to about 100%, or from about 99% to about 100%. The separation may be repeated multiple times to achieve about 100% removal of the second subset of the aptamers from the container or fluidic device that holds the cell.

The aptamer library may be configured to sparsely or densely cover a target cell surface. In some cases, the aptamer library may include a greater number of aptamers than surface protein epitopes on a target cell, and may thereby effectuate competitive binding among aptamers for cell surface binding. In such cases, high mobility aptamers may rapidly bind to the cell surface and then slowly be replaced by slower binding, higher affinity aptamers. Separation of unbound aptamers from the cell at different timepoints can thus return different information regarding surface marker expression. This phenomenon is often referred to as the ‘Vroman effect’, and can be exploited to increase the amount of information generated during surfaceomic analysis. In particular, in some cases, the number of types of aptamers in an aptamer library is greater than the number of surface proteins expressed by a target cell. Similarly, in some cases, an aptamer library includes two or more aptamers that target the same surface protein expressed by a target cell. An aptamer-based analysis method may also be repeated with different incubation times between the combining and the separating to modulate the first subset of the aptamers that couple to the cell.

The aptamers can include the capturable sequences and an aptamer binding portion. In many cases, the capturable sequence is 3′ to the aptamer binding portion so that the nucleic acid barcodes can be extended using the aptamer binding portion and optionally further using additional sequences 5′ to the aptamer binding portion as a template when the capturable sequence is hybridized to the nucleic acid barcode. The aptamers can also include one or more primers and/or barcodes. In some cases, the aptamers include capturable sequences at 3′ ends of the aptamers and primers at 5′ ends of the aptamers. In further cases, the aptamers include barcodes in between the capturable sequence and the primer (e.g., disposed directly between the capturable sequence and an aptamer binding portion or disposed directly between the aptamer binding portion and the primer). The barcode may be an aptamer identity barcode that identifies the aptamer to which the aptamer identity barcode is coupled.

The aptamers can be hybridized to the nucleic acid barcodes. The nucleic acid barcodes can include oligonucleotide capture sequences that are configured to hybridize to the capturable sequences of the aptamers. As an example, the oligonucleotide capture sequences can include polyT sequences that are capable of hybridizing to poly A capturable sequences of the aptamers. The nucleic acid barcodes can include spatial barcode sequences associated with locations of the nucleic acid barcodes on the surface of the fluidic device, so that a nucleic acid barcode or a nucleic acid that contains a complement of the spatial barcode (e.g., is extended using the nucleic acid barcode as a template) can be associated with a particular chamber-enclosed cell within the fluidic device. For example, the method for cellular analysis can include sequencing the spatial barcode sequences or complements of the spatial barcode sequences and associating the first subset of the aptamers with the cell based on the spatial barcode sequences or the complements of the spatial barcode sequences. The nucleic acid barcodes can also include unique molecular identifier sequences, which may allow counts of aptamer sequences to be normalized. The nucleic acid barcodes can also optionally include enzymatically or chemically cleavable moieties, primer binding sites, sequencing adapter sites, or other sequences that facilitate nucleic acid barcode collection, amplification, or analysis.

The method can include dissociating the first subset of the aptamers from the cell. The dissociating may be performed prior to hybridizing the aptamers to the nucleic acid barcodes, and may utilize any method for dissociating an aptamer or other binding agent from the surface of a cell that is known in the art, including lysing the cell, reducing an ionic strength of a solution surrounding the cell, inputting a chaotropic agent into the fluidic device, inputting an organic solvent into the fluidic device, increasing a temperature of the fluidic device, or performing a combination of such methods.

In some cases, the method includes synthesizing one or more chambers that co-enclose the cell, the first subset of aptamers coupled to the cell, and nucleic acid barcodes coupled to a surface of the fluidic device. The one or more chambers can prevent or inhibit diffusion of the aptamers. In particular, the one or more chambers can include pores that are smaller than the aptamers, such that aptamers dissociated from the cell are prevented from diffusing out of the one or more chambers, and are capturable only by the nucleic acid barcodes enclosed by the one or more chambers. Multiple cells in the fluidic device may separately be enclosed within chambers that keep the cells and any aptamers coupled to the cells separate from cells and aptamers in other chambers. Accordingly, in many cases, the first subset of the aptamers are dissociated from the cell after the one or more chambers are synthesized. One or more chambers can also optionally be degraded, for example to allow nucleic acid polymerization or reverse transcription reagents to reach the aptamers after the aptamers couple to nucleic acid barcodes. The one or more chambers may also facilitate separation of the first and second subset of the aptamers. For example, the separating can be subsequent to the synthesizing, wherein the separating includes flowing a liquid into the flow cell causing the second subset of the aptamers in a chamber to flow out of the chamber through a pore in a wall of the chamber while retaining the cell in the chamber, wherein the cell is larger than the pore in the wall of the chamber.

The method can also include one or more additional forms of cellular analysis. The presently disclosed methods provide an advantage over known cellular analysis techniques by allowing multiple forms of analysis to be performed on individual cells in a highly multiplexed manner. Whereas conventional forms of single cell analysis such as fluorescence-activated cell sorting (FACS) are often incapable of generating longitudinal multiomic data, the presently disclosed methods facilitate multiomic analyses by enclosing individual cells or collections of cells within chambers that are configured to retain the cells through wash steps. Accordingly, multiple types of data, including surface proteomic data generated using aptamers, may be generated for individual cells. Additional cellular analyses may be performed in parallel or in tandem with surface proteome analysis, and may include cytotoxicity, proliferative rate, proliferative capacity (i.e., the number of progenitor cells that a cell is capable of producing), transcriptome, genome, soluble factor secretion, cellular morphology, and the like.

In particular cases, the method further includes analyzing cellular nucleic acids from the cell. Such a method can include releasing the cellular nucleic acids from the cell; capturing the cellular nucleic acids or fragments of the cellular nucleic acids on additional nucleic acid barcodes coupled to the surface of the fluidic device; and i) extending the additional nucleic acid barcodes using the cellular nucleic acids as templates and sequencing the extended additional nucleic acid barcodes, ii) extending the cellular nucleic acids using the additional nucleic acid barcodes as templates and sequencing the extended cellular nucleic acids, or iii) a combination thereof. In many cases, cellular nucleic acid release from the cell is concurrent with aptamer dissociation from the cell, and the first subset of aptamers and the cellular nucleic acids are captured on the nucleic acid barcodes, extended, and sequenced simultaneously. As with the first subset of the aptamers, the cellular nucleic acids may be trapped by one or more chambers that co-enclose the cell, such that the cellular nucleic acids primarily or exclusively hybridize to nucleic acid barcodes disposed within the one or more chambers. The cellular nucleic acids can include mRNA, fragmented gDNA, guide RNA, or a combination thereof. In many cases, the cellular nucleic acids include mRNA, such that the sequencing provides transcriptomic information for the cell. In further cases, the cellular nucleic acids include guide RNA, the guide RNA includes a reporter sequence corresponding to a genetic edit for generating a surface protein from the cell, and the method further includes associating the aptamer coupled to the cell with an identity of the surface protein for the cell. However, the guide RNA may also include a reporter sequence that corresponds to a genetic edit indirectly related to the surface protein for the cell, such as a knock-out or isoform gene edit that affects surface proteomic profile, or unrelated to the surface protein for the cell.

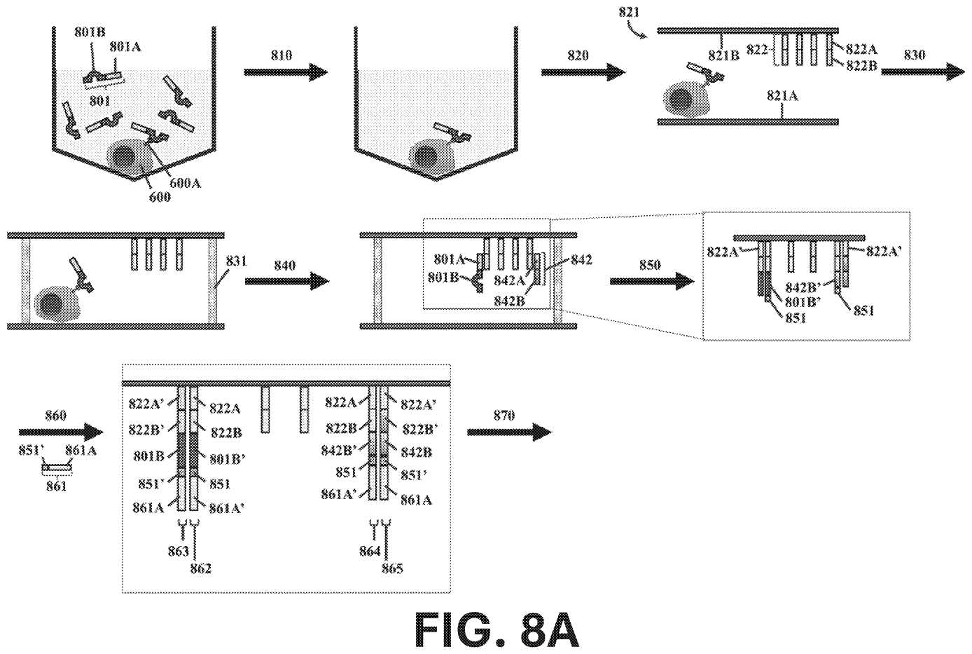

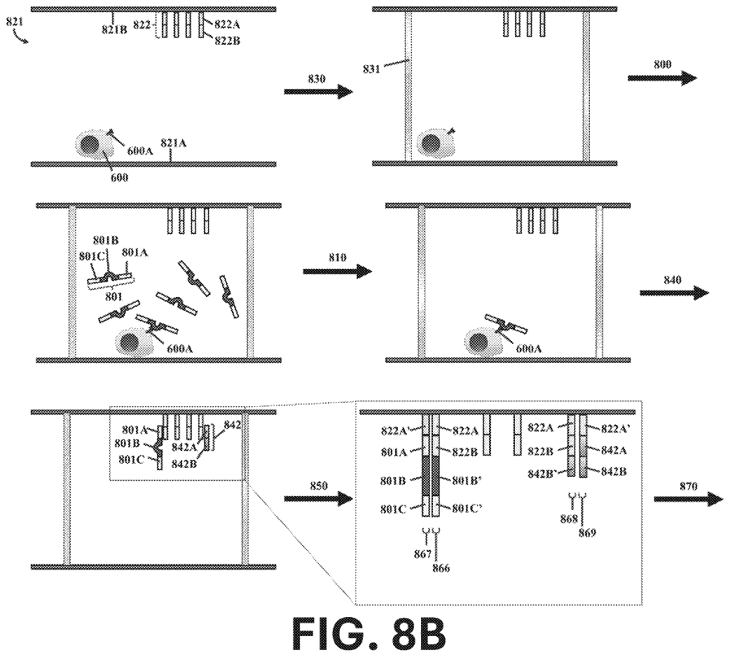

An example of a method for aptamer-based surfaceomic analysis is depicted in FIG. 8A. In this method, aptamers 801 are combined with a cell 600 in a test tube or a similar container such as a well. Each aptamer 801 includes an aptamer binding portion 801B that has an affinity for a surface protein and a capturable sequence 801A such as a poly A tail. The aptamers 801 can include further nucleic acid sequences (e.g., capturable sequences, barcodes, or unique molecular identifiers) or chemical modifications (e.g., blocking groups that prevent extension, binding agents such as biotin or antibody FAB regions, or fluorescent dyes or other detectable moieties) not included in FIG. 8A. The aptamers may also optionally be coupled to a substrate such as a bead. In such cases, each aptamer coupled to a bead may be identical, and the bead may be detectable or may be coupled to a detectable moiety such as a fluorophore that facilitates detection and identification.

Although not shown in FIG. 8A, in some cases, only a portion of the aptamers 801 include a chemical modification. For example, a first portion of a group of aptamers 801 may include a detectable dye, and a second portion of the group of aptamers 801 may not include the detectable dye. Similarly, a first portion of a group of aptamers 801 may include the capturable sequence 801A, and a second portion of the group of aptamers 801 may not include the capturable sequence 801A. In such cases, only the first portion of the group of aptamers that contain the capturable sequences may be captured on nucleic acid barcodes 822, extended in step 850, and sequenced in step 870 as described in FIG. 8A and elsewhere herein.

In continued reference to the method depicted in FIG. 8A, a first subset of the aptamers 801 couple to surface proteins 600A on the cell 600 and a second subset of the aptamers do not couple to the cell. In some cases, the first subset of aptamers that bind to the cell 600 can reflect a competitive binding process, wherein the second subset of aptamers or a portion of the second subset of aptamers are also capable of coupling to surface proteins 600A on the cell 600, but are blocked by the first subset of aptamers (which occupy sites on surface proteins 600A on the cell 600). As a first example, the aptamers 801 may include multiple chemically and/or structurally distinct aptamers that target the same surface protein 600A or surface protein epitope on the cell 600. As a further example, the population of aptamers 801 may be capable of saturating the surface of the cell 600. The unbound second subset of aptamers are then separated in step 810 from the cell, for example through filtering, centrifugation, washing, magnetic bead pull down, or a combination thereof.

The cell and first subset of aptamers coupled to the cell are then input in step 820 into a fluidic device. The fluidic device 821 includes a bottom surface 821A and a top surface 821B opposing the bottom surface. In this example, nucleic acid barcodes 822 are coupled to the top surface 821B. Each nucleic acid barcode 822 contains a spatial barcode sequence 822A associated with its location within the fluidic device 821 and a capture sequence 822B that is configured to hybridize to the capturable sequence 801A of the aptamers 801. The capture sequence 822B may also be configured to hybridize to one or more nucleic acids from the cell, such as mRNA polyA sequences or barcode sequences coupled to guide RNA.

The cell 600, which is still coupled to the first subset of the aptamers, is co-enclosed in a polymeric chamber 831 with the nucleic acid barcodes 822. The polymeric chamber 831 may be synthesized in step 830 from polymer precursors as further disclosed herein, for example through photopolymerization. The nucleic acid barcodes 822 co-enclosed with the cell 600 within the chamber 831 may share a single spatial barcode sequence 822A that is unique to the polymeric chamber 831 (i.e., are not present in other polymeric chambers in the fluidic device 821). Alternatively, the nucleic acid barcodes 822 that are enclosed within the polymeric chamber 831 may include a plurality of spatial barcode sequences 822A that are each unique to the polymeric chamber 831. Nucleic acids with the spatial barcode sequence 822A or a complement of the spatial barcode sequence 822A′ can thus be associated with the polymeric chamber 831 and cell 600 contained therein.

The first subset of the aptamers may then be released in step 840 from the cell. The aptamers may diffuse within the polymeric chamber 831 but, under certain circumstances, may be inhibited from crossing out of the polymeric chamber. In particular, the polymeric chamber 831 may have a pore size that is smaller than the hydrodynamic radii of the aptamers 801, and may thereby block or significantly slow down the aptamers from diffusing across polymer matrix walls of the polymeric chamber. Accordingly, in this example, the first subset of aptamers are capable of diffusing to the nucleic acid barcodes 822 enclosed within the chamber 831, but are not capable of diffusing to nucleic acid barcodes outside of the polymeric chamber (i.e., in other portions of the fluidic device 821). The first subset of aptamers hybridize to the nucleic acid barcode capture sequences 822B through their capturable sequences 801A.

Cellular nucleic acids 842 may also optionally be released 840 from the cell 600. In many embodiments, the aptamers 801 and cellular nucleic acids 842 are released 840 through lysis. The cellular nucleic acids 842 may include protein encoding sequences 842B as well as capturable sequences 842A that hybridize to the capture sequences of the nucleic acid barcodes. In alternative embodiments, the cellular nucleic acids 842 include barcode sequences (e.g., a short sequence associated with a particular plasmid or genetic edit) or a non-protein encoding RNA such as regulatory RNA, ribosomal RNA, snRNA, snoRNA, gRNA, antisense RNA, CRISPR RNA, siRNA, or miRNA, in place of or in addition to the protein encoding sequences 842B. While FIG. 8A depicts cell lysis that releases the first subset of aptamers and nucleic acids from the cell, alternative methods may only release the first subset of aptamers from the cell. For example, the aptamer release 840 can involve a wash step, a buffer exchange, introduction of an organic solvent or chaotropic agent, sonication, or another method that causes the first subset of the aptamers to dissociate from the cell 600 without lysing the cell or causing the cell to release cellular nucleic acids 842.

Returning to FIG. 8A, in a subsequent extension step 850, the aptamers 801 and the cellular nucleic acids 842 are extended using the nucleic acid barcodes 822 as templates, while the nucleic acid barcodes can extend using the aptamers and cellular nucleic acids as templates. In various embodiments, cellular nucleic acids can include DNA, RNA, mRNA, gRNA, or combinations thereof. In this example, when the cellular nucleic acid is RNA, the extension can be performed with a reverse transcriptase that generates overhangs at the 3′ ends of extended nucleic acid barcodes. The nucleic acid barcodes may be further extended 860 using a template switch oligonucleotide 861 as a template. The template switch oligonucleotide 861 may include a complement of the overhang 851′ and a primer sequence 861A. The template switch oligonucleotides 861 may optionally be extended using the nucleic acid barcodes as templates, for example by a strand-displacing reverse transcriptase. When the aptamer 801 includes DNA, the extension can be performed with a DNA polymerase or a reverse transcriptase that also yields an overhang 851 (such as an oligocytosine) that can be coupled to the template switch oligonucleotides 861 or an overhang 851 (such as a single adenosine or guanosine) that can be utilized to direct sticky-end ligation. Following this step, a first extended nucleic acid barcode 862 (extended using the aptamer as a template) may include, from 5′ to 3′, (1) the spatial barcode sequence 822A, (2) the capture sequence 822B, (3) the complement of the aptamer binding portion 801B′, (4) the overhang 851, and (5) a complement of the template switch oligonucleotide primer sequence 861A′. Similarly, a first extended template-switch oligonucleotide 863 (extended using the first extended template-switch oligonucleotide 863 as a template) may include, from 5′ to 3′, (1) the template switch oligonucleotide primer sequence 861A, (2) a complement of the overhang 851′, (3) the aptamer binding portion 801B, (4) a complement of the capture sequence 822B′, and (5) a complement of the spatial barcode sequence 822A. A second extended nucleic acid barcode 864 (extended using the cellular nucleic acid 842 as a template) may include, from 5′ to 3′, (1) the spatial barcode sequence 822A, (2) the capture sequence 822B, (3) a complement of the protein encoding sequences 842B′, (4) the overhang 851, and (5) a complement of the template switch oligonucleotide primer sequence 861A′. A second extended template-switch oligonucleotide 865 (extended using the second extended nucleic acid barcode 864 as a template) may include, from 5′ to 3′, (1) the template switch oligonucleotide primer sequence 861A, (2) a complement of the overhang 851′, (3) the protein encoding sequences 842B′, (4) a complement of the capture sequence 822B′, and (5) a complement of the spatial barcode sequence 822A. The extended aptamers, the extended cellular nucleic acids, the extended nucleic acid barcodes, the extended template-switch oligonucleotides, or a combination thereof can be eluted from the fluidic device and sequenced in step 870.

While FIG. 8A illustrates aptamers 801 that include a capturable sequence 801A at a first end and an aptamer binding portion 801B at a second end, the method depicted in FIG. 8A may also be performed with aptamers 801 that include an additional sequence. The additional sequence may be on an opposite end of the aptamer binding portion 801B as the capturable sequence 801A. This motif is depicted in FIG. 8B, which includes aptamers 801 that have, in order, a capturable sequence 801A, an aptamer binding portion 801B, and a primer sequence 801C. When the primer sequence 801C is present, aptamers 801 are extended using nucleic acid barcodes 822 as templates and nucleic acid barcodes are extended using aptamers as templates, the extended aptamers or the extended nucleic acid barcodes may not need to undergo template-switch oligonucleotide extension or terminal sequence ligation prior to amplification or sequencing, as the primer 801C can serve as the same function(s) as primer sequence 861A of the template switch oligonucleotide 861 (or of an end-ligated primer not shown in FIG. 8A).

FIG. 8B illustrates an alternative method for analyzing a cell surface proteome with aptamers. In this method, a cell 600 is input into the fluidic device before it is combined with aptamers 801. A polymeric chamber 831 is synthesized in step 830 in the fluidic device 821 to co-enclose the cell 600 with the nucleic acid barcodes 822. As disclosed further herein, the fluidic device 821 can include a plurality of discrete spots that include nucleic acid barcodes 822 with spatial barcode sequence 822A uniquely associated with the discrete spot in which they are located. The spatial barcode sequence 822A or sequences enclosed in the chamber 831 may be uniquely associated with the chamber, and may be distinguishable from spatial barcode sequences in other portions of the fluidic device or enclosed by other chambers. While the cell 600 is in the chamber 830, the cell may be perturbed, for example by contacting the cell 600 with a drug (e.g., diffusing a drug through pores in polymeric walls of the chamber), exposing the cell 600 to secreted factors from another cell in the fluidic device, allowing the cell 600 to interact with another cell in the fluidic device, exposing the cell to a condition such as temperature, salinity, dissolved gas concentration, nutrient concentration (e.g., availability or starvation), or a combination thereof. It is worth noting that while a single cell 600 is in the chamber 830 depicted in FIG. 8B, in some methods, a plurality of cells may be enclosed within a single chamber. In such methods, aptamer profiling may detect or characterize an interaction between two or more cells within a single chamber 830.

Following polymeric chamber synthesis 830, aptamers 801 may be input into the fluidic device 800. The aptamers 801, which may be capable of slowly diffusing through the polymer matrix walls of the polymeric chamber 831, may be incubated in the fluidic device 821 for a length of time that is sufficient for the aptamers to diffuse into the polymeric chamber. In this example, the aptamers 801 include, from 5′ to 3′, a capturable sequence 801A, an aptamer binding portion 801B, and a primer sequence 801C. A first subset of the aptamers couple to surface proteins on the surface of the cell, while a second subset of the aptamers do not couple to the surface of the cell. The second subset of the aptamers are then separated 810 from the cell, for example through a wash step. The aptamers 801 are then coupled in step 840 to the nucleic acid barcodes 822, extended in step 850, and sequenced in step 870. Cellular nucleic acids 842 released from the cell 600, for example during a lysis step, may also be captured in step 840 and extended 850. As detailed in Example 1, the aptamers 801 may be stable against nucleic acid strand replacement (i.e., may remain coupled to the nucleic acid barcodes 822) following release of cellular nucleic acids 842. Aptamer 801 retention on nucleic acid barcodes 822 may be promoted by having an excess of nucleic acid barcodes 822 within a chamber 830 (or other type of compartment such as a well or droplet). For example, a chamber 830 (or other type of compartment) may include an at least about 2:1, at least about 10:1, at least about 100:1, at least about 1000:1, at least about 10000:1, about 2:1 to about 10:1, about 2:1 to about 100:1, about 2:1 to about 1000:1, about 2:1 to about 10000:1, about 10:1 to about 100:1, about 10:1 to about 1000:1, about 10:1 to about 10000:1, about 100:1 to about 1000:1, about 100:1 to about 10000:1, or about 1000:1 to about 10000:1 excess nucleic acid barcodes 822 relative to aptamers 801 and capturable cellular nucleic acids 842 (e.g., cellular nucleic acids 842 that have a melting temperature from the nucleic acid barcodes 822 of at least about 50° C. when in 1×pH 7.4 phosphate buffered saline). Similarly, the chamber 830 (or other type of compartment) may contain at least about 10⁶, at least about 10⁷, at least about 10⁸, at least about 10⁹, at least about 10¹⁰, about 10⁶ to about 10⁷, about 10⁶ to about 10⁸, about 10⁶ to about 10⁹, about 10⁶ to about 10¹⁰, about 10⁷ to about 10⁸, about 10⁷ to about 10⁹, about 10⁷ to about 10¹⁰, about 10⁸ to about 109, about 10⁸ to about 10¹⁰, or about 10⁹ to about 10¹⁰ nucleic acid barcodes 822.

Following extension 850, a first extended nucleic acid barcode 866 (extended using the aptamer 801 as a template), may include, from 5′ to 3′, (1) the spatial barcode sequence 822A, (2) the capture sequence 822B, (3) a complement of the aptamer binding portion 801B′, and (4) a complement of the primer sequence 801C′, The extended aptamer 857 may include, from 5′ to 3′, (1) the primer sequence 801C, (2) the aptamer binding portion 801B, (3) the capturable sequence 801A, and (4) a complement of the spatial barcode sequence 822A′. A second extended nucleic acid barcode 868 (extended using the cellular nucleic acid 842 as a template) may include, from 5′ to 3′, (1) the spatial barcode sequence 822A, (2) the capture sequence 822B, and (3) a complement of the cellular nucleic acid protein encoding 842B′. The extended cellular nucleic acid 869 can include, from 5′ to 3′, the protein encoding 842B, (2) the cellular nucleic acid capturable sequence 842A, and (3) a complement of the spatial barcode sequence 822A′. The extended nucleic acid barcodes 866, 868, the extended aptamer 867, the extended cellular nucleic acid 869, or a combination thereof can be sequenced 870 as described for FIG. 8A, extended in step 850, and sequenced in step 870 as described for FIG. 8A.

The aptamer-based analyses disclosed herein can also be performed in droplets, wells (e.g., microwells and nanowells) and other types of partitions. As an example, a method for cellular analysis consistent with the present disclosure can include combining aptamers with a cell, wherein a first subset of the aptamers couple to the cell and a second subset of the aptamers do not couple to the cell; separating the second subset of the aptamers from the cell; forming a droplet, wherein the droplet comprises the cell and nucleic acid barcodes, and the cell is coupled to the first subset of the aptamers; hybridizing first barcode sequences of the first subset of the aptamers to the nucleic acid barcodes; extending the nucleic acid barcodes to generate extended nucleic acid barcodes or extending the first subset of the aptamers to generate extended aptamers; collecting the extended nucleic acid barcodes or the extended aptamers from the droplet; and sequencing the extended nucleic acid barcodes or the extended aptamers.

The nucleic acid barcodes can include oligonucleotide capture sequences that are configured to hybridize to primer or barcode sequences on the aptamers, and optionally to cellular nucleic acids such as gRNA, gDNA fragments, or mRNA. The nucleic acid barcodes may be coupled to a solid substrate within the droplet such as a bead. The nucleic acid barcodes can include barcode sequences associated with the droplet. For example, when the nucleic acid barcodes are coupled to beads, each nucleic acid barcode can include a barcode sequence that is unique to the bead to which it is coupled, and each droplet can include at most one bead, ensuring that each nucleic acid barcode within a particular droplet includes a barcode sequence that is uniquely associated with the droplet. The nucleic acid barcodes can also include unique molecular identifier sequences to allow normalization of sequencing counts.

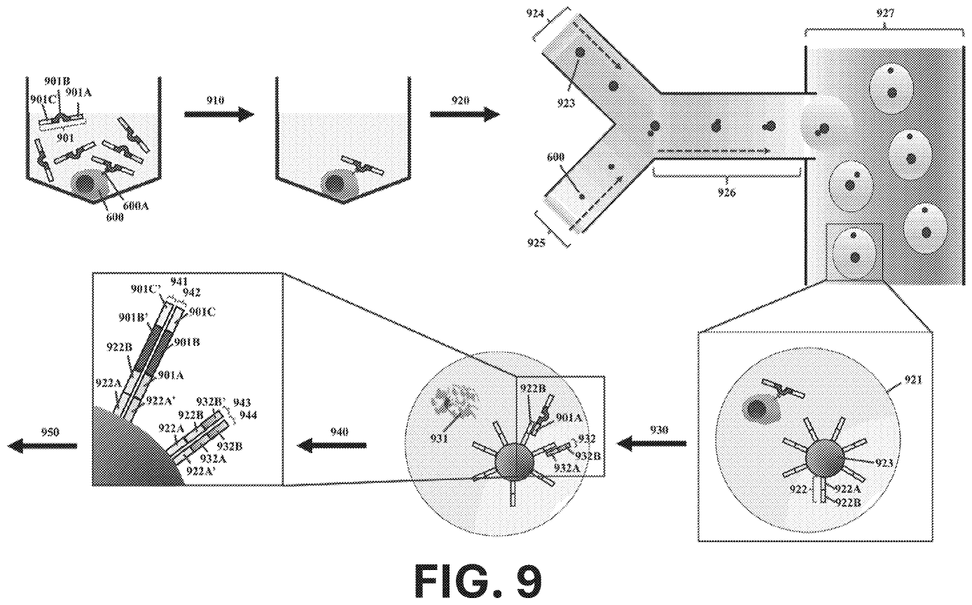

An example of a droplet-based surfaceomic analysis method is depicted in FIG. 9. As in FIG. 8A, aptamers 901 are combined with a cell 600 in a test tube or a similar container. Each aptamer 901 includes an aptamer binding portion 901B that has an affinity for a surface protein, a capturable sequence 901A that is 5′ to the aptamer binding portion, and a primer 901C that is 3′ to the aptamer binding portion. A first subset of the aptamers couple to surface proteins on the cell 600A and a second subset of the aptamers do not couple to the cell. The unbound second subset of aptamers are then separated 910 from the cell. The cell and first subset of aptamers coupled to the cell are then co-enclosed with a particle 923 in a droplet 921.

Next, a droplet 921 can be formed 920 around the cell 600 and a bead 923. In this example, the droplet 921 is formed through a flow focusing method, wherein the cell 600, still coupled to the first subset of aptamers, is flowed through a first inlet channel 925 and the bead 923 is flowed through a second inlet channel 924. Aqueous media streams in the first and second inlet channels 924, 925 flow into a flow focusing channel 926, where the cell 600 and bead 923 combine and then flow into a droplet channel or chamber 927 that contains an oil or other liquid that is immiscible with the aqueous media flowing from the flow focusing channel 926, thereby causing the aqueous media to form into a droplet 921 around the cell 600 and bead 923. Channel 927 can have a flowing oil phase to sweep the formed droplets containing a bead and a cell for subsequent processing and sequencing steps. The aqueous media streams from the first and/or second inlet channels can include lysis reagents, nucleic acid extension reagents (e.g., reverse transcriptase), template switch oligo, a reducing agent for cleaving oligonucleotides from the bead, dNTPs, and optional further reagents for performing a reaction or process within a droplet 921. In various embodiments, the lysis reagent can be inputted with the beads via the second inlet channel so that the lysis reagent is exposed to the cell upon formation of the droplet. The rate at which cells and beads are input into the first and second input channels 924, 925 can be calibrated to limit the formation of droplets with more than one cell or more than one bead.

The bead 923 may contain nucleic acid barcodes 922 that each contain a barcode sequence 922A and a capture sequence 922B. The barcode sequence may be unique to the bead 923 (and thus to the droplet 921 when the droplet contains one bead). Each nucleic acid barcode 922 on the bead 923 may contain the same barcode sequence 922A. The capture sequences 922B of the nucleic acid barcodes 922 may be configured to hybridize to the capturable sequence 901A of the aptamers 901 and optionally to cellular nucleic acids 932 from the cell 600. The bead 923 may optionally be dissolved after it is enclosed within the droplet 921 (not shown in FIG. 9).

The cell 600 may be lysed 930 (thereby converting the cell 600 into a lysed cell 931) to release the aptamer 901 and optionally to release a cellular nucleic acid 932. The cellular nucleic acid can include a protein encoding sequence 932B and a capturable sequence 932A, wherein the capturable sequence 932A may hybridize to a capture sequence 922B of a nucleic acid barcode 922. The aptamer 901 may also hybridize to a nucleic acid barcode 922 coupled to the bead 923. For example, as depicted in FIG. 9, the capturable sequence 901A of the aptamer 901 may hybridize to a capture sequence 922B of a nucleic acid barcode 922.

The cellular nucleic acid 932, the aptamer 601, and the nucleic acid barcodes 922 can then be extended 940 within the droplet 921, thereby generating an extended aptamer, an extended cellular nucleic acid, and extended nucleic acid barcodes. The extended aptamer 942 can include, in order, a complement of the barcode sequence of the nucleic acid barcode 922A′, the capturable sequence 901A, the aptamer binding portion 901B, and the primer 901C. The extended cellular nucleic acid 944 can include, in order, a complement of the barcode sequence of the nucleic acid barcode 922A′, the capturable sequence 932A, and the protein encoding sequence 932B. A first extended nucleic acid barcode 941 (that was extended using the aptamer 901 as a template) can include, in order, the barcode sequence 922A, the capture sequence 922B, a complement of the aptamer binding portion 901B′, and a complement of the primer 901C′. A second extended nucleic acid barcode 943 (that was extended using the cellular nucleic acid 932 as a template) can include, in order, the barcode sequence 922A, the capture sequence 922B, and a complement of the protein encoding 932B. The extended nucleic acid barcode, the extended cellular nucleic acid, the extended aptamer, or a combination thereof can be sequenced 950.

### Secretomic Analysis

Further disclosed herein are methods for measuring protein secretions from cells. Such methods can include inputting a cell into a fluidic device, wherein the fluidic device comprises an aptamer, wherein the aptamer is coupled to the fluidic device, wherein the cell releases a protein; coupling the protein to the aptamer; and detecting the protein coupled to the aptamer, wherein the detecting comprises coupling a binding agent to the protein coupled to the aptamer, and detecting a detectable moiety coupled to the binding agent. In some cases, the protein is a cytokine. However, the protein may be an extracellular protease, lipase, phosphatase, transport protein, or other secreted protein. In other cases, the protein is an intracellular protein, released from the cell during poration or lysis. Optionally, mRNA or guide RNA from the cell can be detected and sequenced allowing the protein secretion to be correlated with mRNA profile or guide RNA associated with a gene edit.

The binding agent can include an antibody or an antibody fragment. Alternatively, the binding agent can include an additional aptamer that is configured to couple to the protein. In general, the binding agent and aptamer couple to different epitopes of the protein, and are therefore capable of simultaneously binding to the protein.

In many cases, the detecting includes detecting an optical signal from the binding agent or from a dye or other optically active species coupled to the binding agent. For example, the detectable moiety can include a dye, and the detecting can include collecting an optical signal from the dye. Alternatively, the detectable moiety can include a fluorescent protein such as green fluorescent protein, and the detecting can include collecting a fluorescence signal from the fluorescent protein.

Alternatively, the detectable moiety includes a nucleic acid sequence of an oligonucleotide coupled to the binding agent, and the detecting includes detecting the sequence. For example, in some cases, the detectable moiety includes a nucleic acid sequence of an oligonucleotide coupled to the binding agent, and the detecting includes releasing the oligonucleotide from the binding agent; capturing the oligonucleotide on a nucleic acid barcode coupled to the surface of the fluidic device; and extending the nucleic acid barcode using the oligonucleotide as a template and sequencing the extended nucleic acid barcode, extending the oligonucleotide using the nucleic acid barcode as a template and sequencing the extended oligonucleotide, or a combination thereof.

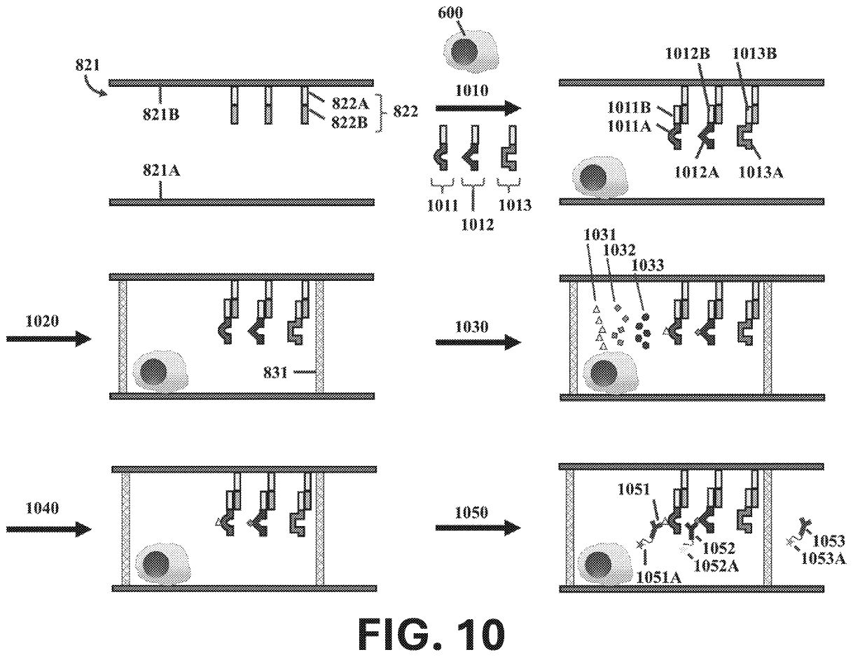

An example of a method for aptamer-based cell secretome analysis is depicted in FIG. 10. This method may utilize a fluidic device 821 with nucleic acid barcodes 822 coupled to a top surface 821B of the fluidic device. However, alternate fluidic device designs in which nucleic acid barcodes 822 are coupled to a bottom surface 821A of the fluidic device are also suitable for this method. Each nucleic acid barcode 822 contains a capture sequence 822B and optionally further contains a barcode sequence 822A, such as a spatial barcode sequence associated with the nucleic acid barcode's location in the fluidic device 821 or a unique molecular identifier sequence.

A cell 600 and multiple aptamers 1011, 1012, 1013 may be input 810 into the fluidic device 821, either concurrently or sequentially in step 1010. The aptamers 1011, 1012, 1013 include capturable sequences 1011B, 1012B, 1013B that hybridize to the capture sequences 822B of the nucleic acid barcodes 822 and aptamer binding portions 1011A, 1012A, 1013A that are configured to couple to secreted proteins. The capturable sequences 1011B, 1012B, 1013B may alternatively hybridize to spatial barcode sequences of the nucleic acid barcodes 822, and may thereby be spatially addressed to particular locations within the fluidic device 821. In various embodiments, multiple aptamers 1011, 1012, 1013 may be attached to the flow cell prior to testing with a cellular sample. The aptamer attachment can be part of a prior manufacturing process before providing modified flow cells to an end user. Under certain conditions, multiple aptamers 1011, 1012, 1013 may be coupled directly to the flow cell or to a nucleic acid barcode attached to a substrate layer of the flow cell where the nucleic acid barcode includes a positional barcode and unique molecular identifier. Alternatively, the aptamers may be coupled to a substrate other than a fluidic device surface such as a bead, a nanomaterial such as a quantum dot or a nanorod, or a protein or protein complex such as albumin. In such cases, the substrate may be optically detectable or coupled to an optically detectable moiety.

A polymeric chamber 831 can be synthesized in step 1020 to co-enclose the cell 600 with the aptamers 1011, 1012, 1013. The cell 600 may then secrete multiple proteins 1031, 1032, 1033 in step 1030. The polymer matrix walls of the polymeric chamber 831 may trap the secreted proteins 1031, 1032, 1033 within the chamber, or may inhibit diffusion of the secreted proteins out of the polymeric chamber. In the example depicted in FIG. 10 at step 1040, a first secreted protein 1031 and a second secreted protein 1032 couple to first and second aptamers 1011 and 1012, respectively, while a third secreted protein 1033 does not couple to an aptamer, and a third aptamer 1013 does not couple to a secreted protein (e.g., the third aptamer 1013 may couple to a fourth protein that is not secreted by the cell 600, and the third secreted protein 1033 may couple to a fourth aptamer that is not present in the fluidic device 821). However, as disclosed further herein, the fluidic device may be loaded with any number of aptamers, for example between 1 and 1000 aptamers. Secreted proteins that are not coupled to aptamers can then be removed 1040 from the fluidic device 821, for example in one or more wash steps. In various embodiments, capture sequence 822B can be configured to capture particular types of aptamers to particular locations within the flow cell.

The fluidic device 821 can then be loaded 1050 with detection antibodies 1051, 1052, 1053 in step 1050. Each detection antibody 1051, 1052, 1053 may be capable of forming sandwich complexes with proteins coupled to the aptamers 1011, 1012, 1013 by coupling to different epitopes on a protein. During this step, a first subset of the detection antibodies couple to secreted proteins that are coupled to aptamers in the fluidic device 821, while a second subset of the detection antibodies that do not bind to secreted proteins can be removed from the fluidic device, for example in one or more wash steps. In this example, a first detection antibody 1051 couples to the first secreted protein 1031 that is coupled to the first aptamer 1011 coupled to the top surface 821B of the fluidic device 821, a second detection antibody 1052 couples to the second secreted protein 1032 that is coupled to the second aptamer 1012 coupled to the top surface of the fluidic device. A third detection antibody 1053 is targeted to the third secreted protein 1033, which is not present in the fluidic device. Accordingly, the third detection antibody 1053 is does not couple to a secreted protein and diffuses out of the polymeric chamber 831.

Each detection antibody 1051, 1052, 1053 can be coupled to a dye 1051A, 1052A, 1053A that produces an optical signal associated with the secreted protein targeted by the detection antibody. In this example, optical signals designate the presence of the first and second detection antibodies 1051 and 1052 and the absence of the third detection antibody 1053 in the polymeric chamber 831, and thereby indicate that the cell 600 expressed the first and second secreted proteins 1031, 1032 and did not express a fourth secreted protein targeted by the third aptamer 1013.

While FIG. 10 depicts secreted protein (1031, 1032, 1033) detection, the method may be similarly used to detect intracellular proteins from a cell (600). For example, after a cell is enclosed in step 1020 within a polymeric chamber 831, the cell 600 may be lysed to release intracellular proteins from the cell 600. The polymer matrix walls of the polymeric chamber 831 may trap the intracellular proteins within the polymeric chamber 831, or may inhibit diffusion of the secreted proteins out of the polymeric chamber 831. The intracellular proteins may couple to aptamers (1011, 1012, 1013) within the polymeric chamber 831, and may be detected by coupling detection antibodies 1051, 1052, 1053 to the intracellular proteins and detecting dyes (1051A, 1052A, 1503A) or other detectable moieties that are coupled to the detection antibodies.

As a non-limiting example, methods for detecting secreted proteins may be used for antibody screening. In some cases, antibody-secreting cells (e.g., B cells) may be compartmentalized with aptamers that bind to or mimic antigens of interest. The antibody-secreting cells may secrete antibodies, for example upon activation by co-compartmentalized helper T cells or stimulatory beads. A subset of the cells may secrete antibodies that couple to the aptamers or antigens bound to aptamers within their compartments. Unbound antibodies may be removed from the compartments. Aptamer or antigen-bound antibodies may then be detected, for example by inputting fluorescently labeled antibody-specific binders (e.g., IgG-targeting aptamers or antibodies) into the compartments to couple to aptamer or antigen-bound antibodies. The antibody secreting cells within these compartments can then be analyzed within the compartments, collected, or a combination thereof. The antibodies secreted by select cells may be identified, for example, by sequencing antibody-encoding mRNA from the cells.

### Fluidic Device and Kit Claims

Further disclosed herein are fluidic devices for performing one or more methods disclosed herein. Such a fluidic device can include a surface comprising a plurality of discrete spots, wherein each discrete spot of the plurality of discrete spots includes: i) nucleic acid barcodes that include spatial barcode sequences associated with the discrete spot and nucleic acid capture sequences, and ii) aptamers. In many aspects, each discrete spot of the plurality of discrete spots includes a single type of aptamer. In other aspects, one or more discrete spots of the plurality of discrete spots includes two or more types of aptamers. In particular aspects, the aptamers are targeted to secreted or intracellular proteins. In other aspects, the aptamers are targeted to cell surface proteins. In various embodiments, discrete spots may be referred to as discrete spots.

### Kits

The present application also provides kits that facilitate one or more of the methods disclosed herein. The kit may include i) a fluidic device that includes a surface with a plurality of discrete spots, wherein each discrete spot of the plurality of discrete spots includes nucleic acid barcodes that include a spatial barcode sequence associated with the discrete spot and a nucleic acid capture sequence; and ii) a composition that includes a plurality of aptamers, wherein each aptamer of the plurality of aptamers includes a nucleic acid sequence complementary to a nucleic acid capture sequence of the plurality of discrete spots. Alternatively, the kit may include i) a fluidic device that includes a surface with a plurality of discrete spots, wherein each discrete spot of the plurality of discrete spots includes nucleic acid barcodes that include a spatial barcode sequence associated with the discrete spot and a nucleic acid capture sequence; and ii) a composition that includes a plurality of aptamers, wherein each aptamer of the plurality of aptamers includes a nucleic acid sequence complementary to a spatial barcode sequence of the plurality of discrete spots.

### Compartments

The term “compartment,” as used herein, generally, refers to a space or volume that may be suitable to contain one or more species or conduct one or more reactions. The compartment may isolate a space or volume from another space or volume. The compartment may comprise one or more other (inner) compartments. In some aspects, it should be noted that the space or volume of a compartment can be an open space or open volume that is occupied by air or liquid. A compartment can have an open space or open volume that is defined by a wall. In various examples, the open space or open volume is not filled with a polymeric material. The wall can be made of a polymer that has an outer surface and inner surface where the inner surface forms at least a part of the boundary of the open space or open volume and the outer surface forms at least a part of the boundary to the area outside of the compartment.

In some aspects, a compartment includes a chamber. A method disclosed herein can include synthesizing one or more chambers that at least partially enclose the one or more cells in a fluidic device. As detailed further herein, the one or more cells may be combined with an aptamer library prior to or after being input into the fluidic device. Similarly, the one or more cells may be combined with the aptamer library before or after chamber synthesis. The one or more chambers may enclose the one or more cells with nucleic acid barcodes that are configured to capture aptamers from the aptamer library.

In many embodiments disclosed herein, the one or more chambers are comprised of one or more polymer matrices formed with the fluidic space. As used herein, the term “polymer matrix” generally refers to a phase material (e.g. continuous phase material) that comprises at least one polymer. In some embodiments, the polymer matrix refers to the at least one polymer as well as the open interstitial space within the polymer. A polymer matrix may be composed of one or more types of polymers. A polymer matrix may include linear, branched, and crosslinked polymer units. A polymer matrix may also comprise non-polymeric species intercalated within its interstitial spaces not occupied by polymer chains. The intercalated species may be solid, liquid, or gaseous species. For example, the term “polymer matrix” may encompass desiccated hydrogels, hydrated hydrogels, and hydrogels comprising glass fibers. A polymer matrix may comprise a polymerized form of a polymer precursor, which generally refers to one or more molecules that upon activation can trigger or initiate a polymeric reaction. A polymer precursor can be activated by electrochemical energy, photochemical energy, a photon (i.e., light), magnetic energy, or any other suitable energy. As used herein, the term “polymer precursor” includes monomers (e.g., that are polymerized to produce a polymer matrix) and/or crosslinking compounds, which may include photo-initiators, other compounds necessary or useful for generating polymer matrices (e.g., porogen).

In particular embodiments, the one or more chambers comprise a hydrogel. As used herein, the term “hydrogel” can refer to a polymeric material that is not water soluble or is poorly water soluble but can contain water (e.g., at least 10% by weight) when hydrated.

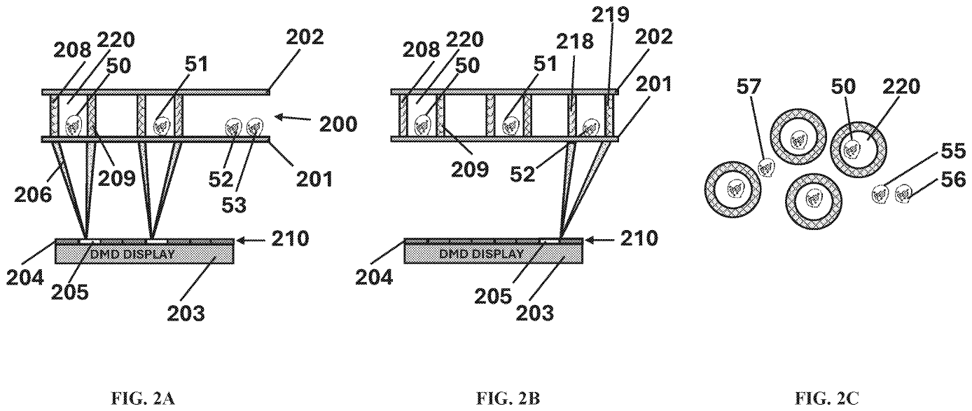

FIGS. 2A-C illustrate an exemplary method for forming a chamber enclosing one or more biological components in a fluidic device disclosed herein. FIG. 2A shows a portion of a system as provided herein (e.g., comprising a fluidic device as disclosed herein) including an energy source forming a polymer matrix around biological components 50 and 51. FIG. 2B shows a polymer matrix being formed around another biological component 52 in a portion of a system as provided herein. FIG. 2C is a top view schematic showing top view of a channel with a polymer matrix formed around a biological component in a system as provided herein. As illustrated in FIGS. 2A-2C, in some embodiments, the one or more chambers have cylindrical shapes that extend from the bottom layer to the top layer of a fluidic device.

With continued reference to FIG. 2A, the channel 200 of the system may include a first surface 201 provided by the bottom layer and a second surface 202 provided by the top layer of the fluidic device. The energy source 203 may comprise one or more energy emitting portions (e.g., an energy emitting portion 205). In some embodiments, the energy source 203 may comprise one or more non-emitting portions (e.g., a non-emitting portion 204). The non-emitting portion 204 does not emit energy for forming a polymer structure, but can be configured at a later time to emit energy. In some embodiments, the emitting portion 205 can emit energy in the form of electromagnetic waves (e.g., microwaves, light, heat, etc.) to at least a portion of the fluidic device. For example, the energy source may comprise an LED array in which individual LEDs can be selectively activated (e.g., act as an energy emitting portion 205) to create light projections with specified patterns.

In further reference to FIG. 2A, the polymer matrix 208, 209, or at least a portion of the polymer matrix 208, 209, may be coupled to the first surface 201, the second surface 202, or both surfaces 201, 202. The polymer matrix 208, 209 may enclose a space 220 that contains a biological component 50. In various embodiments, the polymer matrix 208, 209 may extend from the first surface 201 to the second surface 202 (e.g., through at least a portion of a lumen of the channel 200) such that the polymer matrix surrounds, or substantially surrounds, the biological component 50. FIG. 2B shows polymer matrices 218, 219 formed surrounding the biological component 52. Biological component 53 was separated from biological component 52, and is not present in FIG. 2B. In some embodiments, separation of the biological components is achieved through fluidic pressure, flow pulsation, dielectrophoresis, optothermal flow, or some combination thereof. FIG. 2C provides a top-down view of the channel 200 depicted in FIG. 2B, wherein the cylindrical shapes of the polymer matrix define a compartment 220 surrounding the biological component 50. Additional biological components 55, 56, 57, which are not enclosed within polymer matrices, are shown in FIG. 2C.

In some embodiments, the generation of a polymer matrix within said fluidic device is performed using a spatial light modulator (SLM) (i.e. a spatial energy modulation element that is capable of generating desired light intensity pattern spatially). In some embodiments, the SLM is a digital micromirror device (DMD). In some embodiments, the SLM is a laser beam steered using a galvanometer. In some embodiments, the SLM is liquid crystal based. In some embodiments, the fluidic channel may be coupled to or disposed on a movable stage. In other embodiments, light may be projected to or onto at least a portion of the first fluidic channel to generate one or more polymer matrices. The light may be directed to various parts of the first fluidic channel. The energy source (e.g., light source) may be coupled to the fluidic device via an objective (e.g., a microscope objective or lens). The energy source may be directed to a portion of the fluidic channel (e.g., via a movable objective). In some cases, the light source, the objective, and/or the fluidic channel are movable to allow emission of energy to the fluidic channel so as to generate a pattern on at least a portion of a surface of the fluidic device. The polymer matrix may be formed similarly or complementary to the pattern of energy emission.

In some embodiments, such chambers have annular-like cross-sections. As used herein, the term “annular-like cross-section” means a cross section topologically equivalent to an annulus. In some embodiments, the inner space, or interior, of a chamber has an inner diameter from 5 μm to 500 μm and a volume in the range of from about 1 nanoliter to 100 nanoliters, or from about 1 nanoliter to 10 nanoliters. In some embodiments, the polymer matrix wall has a thickness from about 1 μm (micrometer) to about 30 μm or about 5 to about 15 μm. In some embodiments, a chamber extends from a bottom surface to a top surface of a fluidic device. In some embodiments, a chamber has a height of about 10 μm to about 500 μm. In some embodiments, a polymer matrix wall having an annular-like cross-section has an aspect ratio (i.e., height/width) of 1 or less. In some embodiments, aspect ratio and polymer matrix wall thickness are selected to maximize chamber stability against forces, such as reagent flow through the channel, washings, and the like.

In some embodiments, the generation of a polymer matrix within said fluidic device comprises exposing the one or more polymer precursors to an energy source. In some embodiments, the energy source is a light generating device. In some embodiments, the light generating device generates light ranging from about 350 nm to about 800 nm. In some embodiments, the generation of the polymer matrix comprises from about 1 to about 50 seconds of illumination or from about 2 to about 4 seconds.

Optionally, a first chamber of the one or more chambers can be disposed inside of a second chamber of the one or more chambers. This design can be utilized to separately partition two species (e.g., a cell and a reagent or two cells) within close proximity. This design can also be used to control the timing with which two species are contacted. For example, a method can include forming a first chamber around a cell, flowing a bead (or other assay reagent incapable of diffusing into the first chamber) adjacent to the cell, forming a second chamber surrounding the first chamber and enclosing the bead, and selectively degrading the first chamber to allow the cell and bead to come into contact within the second chamber.

In some embodiments, a functional group can be coupled to one or more chambers. Some non-limiting examples of functional group may include a capture reagent (e.g., pyridinecarboxaldehyde (PCA)), an acrylamide, an agarose, a biotin, a streptavidin, a strep-tag II, a linker, a functional group comprising an aldehyde, a phosphate, a silicate, an ester, an acid, an amide, an aldehyde dithiolane, PEG, a thiol, an alkene, an alkyne, an azide, or a combination thereof. In some cases, the functionalized chamber may be used to capture a biomolecule enclosed therein, thereby trapping the biomolecule in proximity to a biological component (e.g., a cell) enclosed within the chamber. The biomolecule may be produced by the biological component (e.g., secretome from a cell). The functionalized surface of the polymer matrix inside the compartment may be used to capture reagents or molecules from outside the compartment. The functionalized surface may increase surface area covered by a reagent, a molecular sensor, or any molecule of interest (e.g., an antibody).

A fluidic device disclosed herein can include a detector that is configured to detect one or more locations of one or more biological components contained within a channel. Returning to FIGS. 2A-B, in certain embodiments, the energy source 203 can comprise, be coupled to, or be in communication with a detector that detects, or is configured to detect, a location of a biological component in the fluidic device. In some embodiments, the image may be obtained from a camera (e.g., a digital camera, fluorescent imaging camera, etc.). In some embodiments, the camera may be coupled to, connected to, or in communication with the energy source 203. For example, the camera (not shown) may be in electrical communication with the energy source 203. In some embodiments, the energy source 203 may comprise the camera. In various embodiments, the energy source 203 may comprise a microscope (e.g., a fluorescence microscope, a confocal microscope, lens-free imaging system, a transmission electron microscopy (TEM), a scanning electron microscope (SEM), etc.). The microscope may be used to detect one or more positions of one or more biological components (e.g., in combination with the detector).

In some embodiments, one or more chambers has sufficiently large pores to allow movement or transfer of a reagent (e.g., an enzyme, a chemical compound, a small molecule, an antibody, etc.) therethrough, but sufficiently small so as to retain biological components (e.g., cells). Alternatively or additionally (e.g., when two or more chambers are synthesized with different polymer precursors or radiation powers), one or more chambers can have sufficiently small pores to prevent movement or transfer of a reagent and/or biological component (e.g., DNA, RNA, a protein, a cell, etc.) through the walls of the chamber. In some embodiments, the pores have a diameter from 5 nm to 100 nm. In some embodiments, the pores have a diameter smaller than 5 nm. Pore size may be selected based on the sizes of reagents and analytes in an assay. For example, pore size may be modulated by adjusting polymer precursor formulation, porogen size, pH, salt concentration, photopolymerization light duration and light intensity, and the like. In some embodiments, the average diameter of pores of a chamber prevent passage of molecules having a molecular weight of 25 kiloDaltons (kDa) or greater; or having a molecular weight of 50 kDa or greater; or having a molecular weight of 75 kDa or greater; or having a molecular weight of 100 kDa or greater; or having a molecular weight of 150 kDa or greater. In some embodiments, the pores of a chamber are sufficiently large for 150 kDa species such as antibodies, as well as smaller reagents, to flow through, but prohibitively small for cells or beads to flow through. In some embodiments, DNA or RNA retained have lengths that are sequencable using conventional sequencing-by-synthesis techniques. For example, such DNA or RNA comprise at least 50 nucleotides, or in some embodiments, at least 100 nucleotides. In some embodiments, the pores may have an average diameter from 5 nm to 100 nm.

In some cases, the analysis channel may include polymer matrix structures for capturing or trapping a biological component or a molecule or compound produced by the biological component (e.g., prior to introduction of the biological components into the fluidic device). For example, a user may obtain an analysis channel that includes polymer matrix structures. That is, the user may not form the polymer matrix structures. In various cases, the analysis channel may be configured to include polymer matrix structures for capturing or trapping a biological component or a molecule or compound produced by the biological component. For example, in such embodiments, subsequent to introduction of the biological components into the fluidic device and the analysis channel, one or more polymer matrix structures may be formed in the analysis channel. The analysis channel may be configured for a screening process, a library preparation, or another suitable process. In some embodiments, the screening process may be for drug screening, antibiotic screening, culture conditions screening, or CRISPR screening. In certain cases, a plurality of samples may be placed into a plurality of channels. The plurality of samples may be screened against a variety of conditions in other signal-containing channels.

The sealable aperture may be configured to transition from a sealed state to an open state. For example, a sealable aperture may comprise a heat sensitive polymer that can melt, for example, upon receiving heat and render the sealable aperture open. In some cases, the passage of the biological component through the sealable aperture may be inhibited in the sealed state. In certain cases, the passage of the biological component through the sealable aperture may be allowed in the open state. In some cases, the sealable aperture may be sealed with an agarose gel, a temperature-soluble polymer, an N-isopropylacrylamide (NIPAAm) polymer, a wax compound, an alginate, or any other suitable compound or material.

In some aspects, a compartment may be formed by expanding a stimulus-responsive polymer to enclose a biological material. The stimulus-responsive polymer may expand from a contracted state to an expanded state in response to a stimulus such as temperature, osmolarity, pH, or a combination of stimuli thereof. In the contracted state, the stimulus-responsive polymer may form a portion of a compartment that includes an opening through which a biological material such as a cell may flow through. After a biological material flows into the opening, the stimulus responsive polymer may be expanded, which may shrink or close the opening to form a compartment with the biological material trapped therein. The stimulus may be applied in a spatially controlled manner, for example by using light directed from a spatial light modulator to selectively heat (e.g., in the presence of thermoresponsive particles) individual stimulus-responsive polymers within a fluidic device. The stimulus may also be applied throughout a fluidic device or a portion of a fluidic device, for example by heating or replacing a buffer in a fluidic device or an individual fluidic channel within a fluidic device. Examples of stimulus-responsive polymers consistent with the present disclosure include poly(N-isopropylacrylamide) (PNIPAM), poly(N-vinylcaprolactam) (PVCL), poly(acrylic acid) (PAA), poly(methacrylic acid) (PMAA), poly(itaconic acid) (PIA), poly(glutamic acid) (PGA), poly(aspartic acid) (PASP), a poly(β-amino ester) (PBAE), poly(L-histidine) (PLH), poly(2-vinylpyridine) (P2VP), poly(4-vinylpyridine) (P4VP), poly(dimethylaminoethyl methacrylate) (PDMAEMA), poly(diethylaminoethyl methacrylate) (PDEAEMA), poly(allylamine hydrochloride) (PAH), poly(styrene sulfonate) (PSS), poly(acrylamide-co-acrylic acid) (PAM-co-PAA), poly(acrylamide-co-dimethylaminoethyl methacrylate) (PAM-co-PDMAEMA), poly(acrylamide-co-diethylaminoethyl methacrylate) (PAM-CO-PDEAEMA), as well as combinations thereof.

In some cases, the compartments are droplets. A droplet can serve as a reaction chamber for one or more cells. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. In some cases, the droplets are encapsulated in oil. In some cases, the droplets comprise one or more beads. In some cases, each droplet comprises a single bead. A bead can be a gel bead. In some cases, a bead comprises one or more oligonucleotides. The oligonucleotides can be used to capture one or more components from a cell in the droplet. The oligonucleotides can include a barcode unique to the bead which they are disposed on. Within each droplet, a cell can be lysed, and nucleic acids from the cell (including messenger RNA) can bind to a unique barcode on the gel bead, which can then be used to identify the cell of origin. In some cases, the droplets are formed by mixing cells and barcoded gel beads with oil to create the droplets. Enclosing a nucleic acid molecule or a derivative thereof or a cell comprising the nucleic acid molecule or a derivative thereof and one or more reagents may comprise flowing a first phase comprising an aqueous fluid, the cell, and the one or more reagents and a second phase comprising a fluid that is immiscible with the aqueous fluid toward a junction. Upon interaction of the first and second phases, a discrete droplet of the first phase comprising the nucleic acid molecule or a derivative thereof or a cell comprising the nucleic acid molecule or a derivative thereof and the one or more reagents may be formed. In some cases, the compartment may comprise a single cell. The cell may be lysed or permeabilized within the compartment (e.g., droplet) to provide access to the nucleic acid molecule of the cell. In some cases, reagents (including reverse transcription reagents) are also included during droplet formation. One or more reagents may be co-enclosed with a nucleic acid molecule or a derivative thereof or a cell comprising the nucleic acid molecule or a derivative thereof. For example, a nucleic acid molecule or a derivative thereof or a cell comprising the nucleic acid molecule or a derivative thereof may be co-enclosed with one or more reagents selected from the group consisting of lysis agents or buffers, permeabilizing agents, enzymes (e.g., enzymes capable of digesting one or more RNA molecules, extending one or more nucleic acid molecules, reverse transcribing an RNA molecule, permeabilizing or lysing a cell, or carrying out other actions), fluorophores, oligonucleotides, primers, probes, barcodes, nucleic acid barcode molecules (e.g., nucleic acid barcode molecules comprising one or more barcode sequences), buffers, deoxynucleotide triphosphates, detergents, reducing agents, chelating agents, oxidizing agents, nanoparticles, beads, and antibodies. In some cases, a nucleic acid molecule or a derivative thereof, or a cell comprising the nucleic acid molecule or a derivative thereof, may be co-enclosed with one or more reagents selected from the group consisting of temperature-sensitive enzymes, pH-sensitive enzymes, light-sensitive enzymes, reverse transcriptases, proteases, ligase, polymerases, restriction enzymes, nucleases, protease inhibitors, exonucleases, and nuclease inhibitors. Droplets may have overall volumes that are less than about 5000 pL, 2500 pL, 1000 pL, 500 pL, 250 pL, 100 pL, 50 pL, or less.

As described herein, one or more processes may be performed in a compartment, which may be a well. The well may be a well of a plurality of wells of a substrate, such as a microwell of a microwell array or plate, or the well may be a microwell or microchamber of a device (e.g., microfluidic device) comprising a substrate. The well may also be a nanowell. The well may be a well of a well array or plate, or the well may be a well or chamber of a device (e.g., fluidic device). Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid. The well may have a volume of less than 1 milliliter (mL). For instance, the well may be configured to hold a volume of at most 1000 microliters (pL), at most 100 pL, at most 10 pL, at most 1 pL, at most 100 nanoliters (nL), at most 10 nL, at most 1 nL, at most 100 picoliters (pL), at most 10 (pL), or less. The well may be configured to hold a volume of about 1000 pL, about 100 pL, about 10 pL, about 1 pL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 pL, at least 10 pL, at least 100 pL, at least 1000 pL, or more. The well may be configured to hold a volume in a range of volumes listed herein, for example, from about 5 nL to about 20 nL, from about 1 nL to about 100 nL, from about 500 pL to about 100 pL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well. In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a microcapsule, droplet, bead, or other reagent. In some cases, the adjacent microwells may be configured to merge the contents held within, e.g., upon application of a stimulus, or spontaneously, upon contact of the articles in each microwell.

In some cases, cells can be enclosed using optofluidic technology. A fluidic device can include one or more distinct regions, which can be at least partially physically separated from each other. In some cases, these distinct regions may be referred to as “pens.” For example, a fluidic device can include a channel in fluidic communication with a plurality of pens. A pen may be open on a side of the pen that abuts or is adjacent to a channel. Cells can be loaded into the channel. Energy, including light, can be applied to the fluidic device. The projected light can change electrical properties of certain regions of the fluidic device to create electrical fields that push or pull cells. This light energy can be used to pick up single cells or a certain number of cells, move the single cell or cells, and place the single cell or cells in a specific pen within the fluidic device. This can result in a pen of a fluidic device comprising a single cell. Once isolated, cells may remain in the pen and kept alive with controlled media flow. The pens within a fluidic device can be continuously monitored. As described elsewhere herein, any type of assay may be performed on a single cell or multiple cells within a pen (functional assays, imaging assays, morphology assays, proteomic assays, metabolic assays, etc.). Nucleic acids can be released from the cells and contained within the pen.

In some cases, cells are spatially constrained to a unique area of the fluidic device, with or without a physical compartment. In some cases, each of the unique areas is no more than 285,000 μm², 200,000 μm², 150,000 μm², 100,000 μm², 50,000 μm², 25,000 μm², 20,000 μm², 15,000 μm², 12,000 μm², 11,000 μm², 5,000 μm², 1,000 μm², 500 μm², 100 μm², 10 μm², or 1 μm². In some cases, a physical compartment as described elsewhere herein is used to constrain the area a cell can move. In some cases, a cell is constrained to an area without the use of a physical compartment. The present disclosure also provides systems including one or more capture elements for immobilizing and/or compartmentalizing one or more biological components (including cells). The system can include a fluidic device. The fluidic device can include or contain one or more biological components. In some embodiments, the fluidic device can include a first surface (e.g., in a channel and/or chamber of the fluidic device). The fluidic device can include one or more capture elements. The capture elements can immobilize, or be configured to immobilize, at least one of the one or more biological components at a location on or adjacent to the first surface (or any suitable surface). Immobilization or coupling of a biological component to a capture element can form an immobilized biological component. In some embodiments, the one or more capture elements comprise a physical trap (including a divot), a geometric trap, a well, an electrochemical trap, a chemical affinity trap, one or more magnetic particles, an electrophoretic trap, a dielectrophoretic trap, or a combination thereof. In some embodiments, the chemical affinity trap comprises streptavidin, an antibody, or a combination thereof. In some embodiments, the electrochemical trap comprises a gold electrode, a platinum electrode, an indium tin oxide (ITO) electrode, or other suitable electrochemical trap. In some embodiments, the one or more capture elements are disposed in a pattern on the first surface. In some embodiments, the one or more capture elements comprises a well as described elsewhere herein. For example, a cell can be constrained to a specific area of a fluidic device by using a capture element. A biological component (including a cell) can couple to a capture element on a surface, for example. Once coupled to the capture element, the biological component may be constrained to a specific area of the fluidic device. As described elsewhere herein, any number and combination of assays can be performed on a spatially constrained biological component.

### Fluidic Device

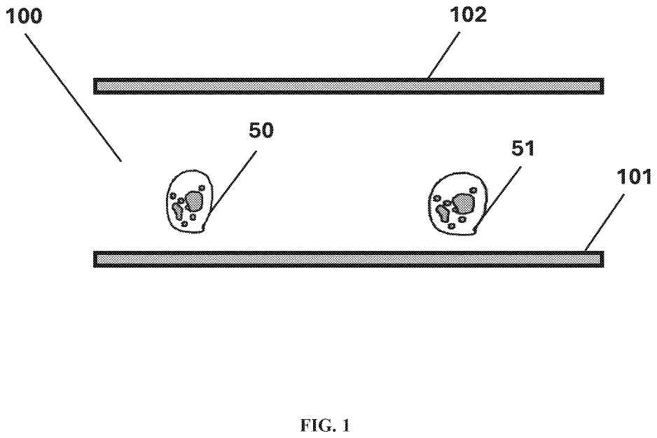

FIG. 1 illustrates cells disposed within a channel 100 of a fluidic device. A channel 100 of a fluidic device can also sometimes be referred to as a “flow chamber,” “flow channel,” or “reaction chamber”), and may receive or be configured to receive a biological sample. FIG. 1 shows a simplified schematic cross-sectional side view illustration of a portion of a channel 100 of a fluidic device. The channel 100 may comprise a first surface 101 and a second surface 102 positioned opposite of one another (e.g., as depicted in FIG. 1). In some embodiments, a middle spacer layer of double-sided adhesive with a cut-out portion can be used to position the first surface 101 and second surface 102 in a facing relationship to at least partly form the flow channel 100. The perpendicular distance between the first surface 101 and the second surface depends in part on the nature and size of the biological components to be analyzed. In some embodiments, such as, those adapted to analyzing mammalian cells, the perpendicular distance between a first surface and a second surface may be in the range of from 10 μm to 500 μm, or in the range of from 50 μm to 250 μm. In some embodiments, the perpendicular distance between a first surface and a second surface may be in the range of from twice the average size of the biological component to be analyzed to five times the average size of the biological component to be analyzed. In some embodiments, the first surface 101 may be a lower surface. In certain embodiments, the second surface 102 may be an upper surface. In some embodiments, the first surface 101 and second surface 102 are substantially parallel, so that the perpendicular distance between them is substantially the same throughout the channel, for example, where chambers are formed. The perpendicular distance may vary by less than +/−10%, 5%, 1%, or 0.1% throughout the channel 100. The channel 100 may receive a biological sample comprising one or more biological components 50, 51. The channel 100 may receive one or more polymer precursors. As illustrated in FIG. 1, the biological components 50, 51 may include cells. However, as discussed herein, the biological components may include tissues, proteins, nucleic acids, etc. In some embodiments, the first surface 101 and/or second surface 102 can be optically transmissive so that visible and UV light can transmit through one or both of the surface for the generation of polymeric hydrogels, imaging of the flow cell, and the measurement of the analyte and biological components.

In certain cases, a channel may have a cross-sectional area that is rectangular, circular, semi-circular, or oval. Accordingly, the channel may have a single, internal surface. In some cases, a channel may have a triangular, square, rectangular, polygonal, or other cross-section. Accordingly, the channel may have three or more internal surfaces. One or more of the internal surfaces may be couple or receive, or be configured to couple or receive, the one or more biological components.

In certain cases, a channel may have a cross-sectional area that is rectangular, circular, semi-circular, or oval. Accordingly, the channel may have a single, internal surface. In some cases, a channel may have a triangular, square, rectangular, polygonal, or other cross-section. Accordingly, the channel may have three or more internal surfaces. One or more of the internal surfaces may be couple or receive, or be configured to couple or receive, the one or more biological components.

The first surface 101, the second surface 102, or both surfaces 101, 102 may be functionalized, for example with a coating. As a non-limiting example, a surface coating may be a surface polymer. Some non-limiting examples of surface coatings may include a capture reagent (e.g., pyridinecarboxaldehyde (PCA)), a functional group to capture one or more moieties (e.g., a chemical moiety), an acrylamide, an agarose, a biotin, a streptavidin, a strep-tag II, a linker, a functional group comprising an aldehyde, a phosphate, a silicate, an ester, an acid, an amide, an alkyne, an azide, an aldehyde dithiolane, or a combination thereof. In various embodiments, the surface coating may include a functional group to capture one or more moieties. For example, the acrylamide, the agarose, etc. may include such a functional group. In certain embodiments, the surface polymer may comprise polyethylene glycol (PEG), a thiol, an alkene, an alkyne, an azide, or combinations thereof. In various embodiments, the surface polymer may comprise a silane polymer derivatized with N-hydroxysuccinimide. In some embodiments, the surface polymer may be functionalized with at least one of an oligonucleotide, an antibody, a cytokine, a chemokine, a protein, an antibody derivative, an antibody fragment, a carbohydrate, a toxin, or an aptamer. In particular embodiments, the surface coating comprises a material for which adherent cells have a binding affinity, such as fibronectin, laminin, poly-L-ornithine, or combinations thereof.

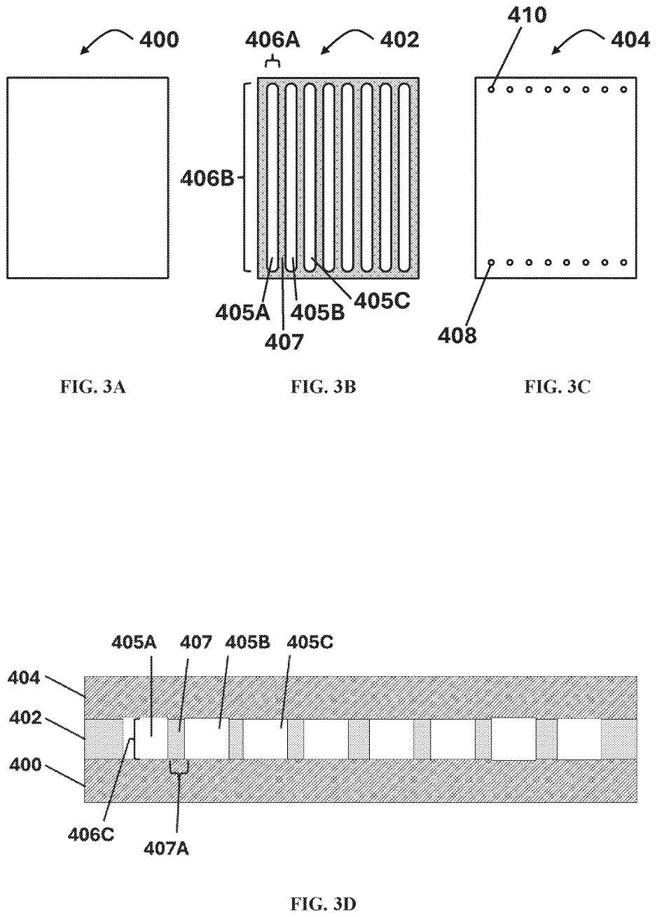

FIGS. 3A-D illustrate an example of a fluidic device consistent with the present disclosure. The channel 100 of FIG. 1 may correspond to any one of the cut-out regions (405A, 405B, 405C) of FIG. 3D. FIGS. 3A-D figures depict the components of a flow cell comprised of a bottom layer 400 (FIG. 3A), a spacer layer 402 (FIG. 3B), and top layer 404 (FIG. 3C). The bottom layer (400) and the top layer (404) can both independently be a glass or plastic material. In some cases, the top layer (404) is optically transparent or translucent. In some cases, the bottom layer (400) is optically transparent or translucent. In many cases, the top layer (404) and the bottom layer (400) are optically transparent or translucent. The spacer layer (402) can be a double-sided pressure sensitive adhesive with one or more cut-out regions (405A, 405B, 405C). In this design, the one or more cut-out regions of the spacer layer are sandwiched between the bottom layer and the top layer to form one or more channels. In various embodiments, the spacer layer (402) includes a core plastic (e.g., PET) layer with pressure sensitive adhesive coating on its top and/or bottom sides that contact the top (404) and bottom (400) layers. Examples of adhesive coatings consistent with the present disclosure include siloxane, silicone, acrylate, acrylamide, polyvinyl, polyurethane, epoxy, polyphenol, polyester, polyamide, polyimide, polytetrafluoroethylene, polyethylene, polypropylene, polycarbamate, polycarbonate, polyacrylic acid, sulfonated polyester, and combinations thereof.

Referring to FIG. 3B, a peripheral portion (407) of the spacer layer (402) provides a boundary for one or more cut-out regions (405A, 405B, 405C) with defined widths (406A) and lengths (406B). In many designs disclosed herein, the spacer layer (402) adheres to the top (404) and bottom (400) layers, such that a cut-out region or plurality of cut-out regions (405A, 405B, 405C) define a channel or a plurality of channels. The spacer layer (402), bottom layer (400), and/or top layer (404) can be water (or optionally more generally liquid) impermeable such that a first aqueous sample in a first cut-out region (e.g., 405A) is isolated from a second aqueous sample in a second cut-out region (e.g., 405B). Biological components and reagents in a first cut-out region (e.g., 405A) may similarly be isolated from other cut-regions (405B, 405C). The one or more cut-out regions (405A, 405B, 405C) can thus define flow cell channels with defined dimensions.

A channel defined by a cut-out region (405A, 405B, 405C) can be operably coupled to an inlet (408) and/or an outlet (410) in the top layer (404), through which, for example, a gas, liquid, sample, or reagent may flow. In various embodiments, the inlet (408) and outlet (410) can both be represented as through holes in the top layer (404). However, the inlet (408) and/or outlet (410) can be disposed within other components of the flow cell, such as the bottom layer (400) or spacer layer (402).

With respect to FIG. 3D, the top layer (404) can have a height of about 0.7 mm and the bottom layer can have a height of about 0.5 mm. The first cut-out region (406) can have a height (406C) defined by the distance between opposing faces of the top layer (404) and the bottom layer (400). The spacer layer can have a height ranging from about 50 microns to about 200 microns, and preferably, from about 70 microns to about 130 microns. The cut-out region can have dimensions of about 10.3 cm length×about 0.7 cm width.

### Polymer Precursors

A channel of a fluidic device (e.g., the channel) can comprise one or more polymer precursors for forming chambers. The polymer precursor may be light activatable. For example, synthesizing one or more chambers can comprise projecting light into the channel with a spatial energy modulating element such that the projected light causes cross-linking of one or more polymer precursors to at least partially form the one or more chambers. As discussed in further detail below, a wide variety of photo-synthesizable gels and degradable gels are available for implementing the systems and methods described herein. In some embodiments, photosynthesized gels are formed using a photo-initiator for radical polymerization. In some embodiments, photo-initiators comprise 2-hydroxy-4′-(2-hydroxyethoxy)-2-methylpropiophenone (Irgacure 2959), lithium phenyl-2,4,6-trimethylbenzoylphosphinate (LAP), or Eosin-Y. In some embodiments, one or more polymer precursors comprise hyaluronic acid, chitosan, heparin, alginate, polyethylene glycol (PEG), multi-arm PEG, poly(ethylene glycol)-b-poly(propylene oxide)-b-poly(ethylene glycol) (PEG-PPO-PEG), poly(lactic acid-co-glycolic acid)-b-poly(ethylene glycol)-b-poly(lactic acid-co-glycolic acid) (PLGA-PEG-PLGA), and poly(vinyl alcohol). In some embodiments, polymer precursors comprise PEG or multi-arm PEG. In some embodiments, the polymer precursors comprise an enzymatically degradable cross-linker. In some embodiments, such enzymatically degradable cross-linker is degradable by an esterase or a peptidase. In some embodiments, polymer precursors comprise a photo-degradable cross-linker. In some embodiments, such photo-degradable cross-linker comprises a nitrobenzyl group. In some embodiments, such photo-degradable cross-linker comprises a coumarin moiety.

In some embodiments, the one or more polymer precursors are added to the fluidic device along with the one or more cells. In various embodiments, the one or more cells and the one or more polymer precursors can be pre-mixed and then added to the fluidic device at the same time; the one or more cells and the one or more polymer precursors can be added to the fluidic device at the same time; the one or more cells can be added to the fluidic device first and then the one or more polymer precursors can be added to the fluidic device second; the one or more cells can be added to the fluidic device first allowing the one or more cells couple to a surface in the fluidic device, and then the one or more polymer precursors can be added to the fluidic device second; or the one or more polymer precursors can be added to the fluidic device first and then the one or more cells can be added to the fluidic device second.

Polymer precursors may be selected from a wide variety of compounds including, but not limited to, polyethylene glycol (PEG)-thiol, PEG-acrylate, acrylamide, N,N′-bis(acryloyl) cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), polylysine, agar, agarose, alginate, heparin, alginate sulfate, dextran sulfate, hyaluronan, pectin, carrageenan, gelatin, chitosan, cellulose, collagen, bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetraacrylate, or combinations or mixtures thereof. Representative crosslinkers useful for polymer synthesis are listed in Tables 1A and 1B. Degradation agents that may be used to degrade chambers formed from particular polymer precursors are indicated in column 4 of Table 1A (column 4).

TABLE 1A

| Precursors | Hydrogels | Crosslinkers | Degradation Agents |
|---|---|---|---|
| Acrylamide | Polyacrylamide | Bis-acryloyl cystamine | DTT/TCEP/THP |
| | | (Structure 1) | |
| PEG-based acryloyl | PEG | Bis(2- | DTT/TCEP/THP |
| | | methacryloly)oxyethyl | |
| | | disulfide (Structure 2) | |
| Dextran-based | Dextran | N,N′-(1,2- | NaIO₄ |
| acryloyl | | Dihydroxylethylene)bis- | |
| | | acrylamide (Structure 3) | |
| Polysacchride-base | Polysaccharide | Structure 4 | NaOH, |
| acryloyl | | | ethanolamine, |
| | | | DTT/TCEP/THP |
| Gelatin-base | Gelatin | Structure 5 | NaOH, |
| acryloyl | | | ethanolamine, |
| | | | nucleophilic bases |
| — | — | Structure 6 | NaOH, alkali, |
| | | | organic bases |
| — | — | Structure 7 | Acid |

TABLE 1B

| Structure | |
|---|---|
| Number | Formula |
| 1 | 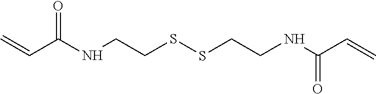 [SMILES: C=CC(=O)NCCSSCCNC(=O)C=C] |
| 2 | 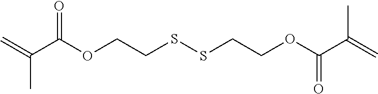 [SMILES: C=C(C)C(=O)OCCSSCCOC(=O)C(=C)C] |
| 3 | 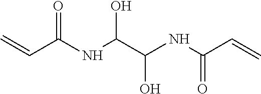 |
| 4 | 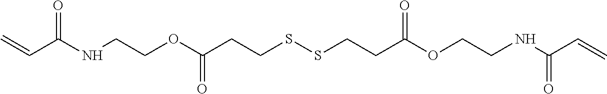 [SMILES: C=CC(=O)NCCOC(=O)CCSSCCC(=O)OCCNC(=O)C=C] |
| 5 | 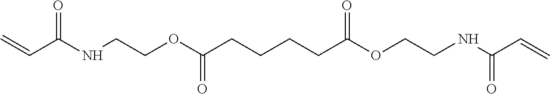 [SMILES: C=CC(=O)NCCOC(=O)CCCCC(=O)OCCNC(=O)C=C] |
| 6 | 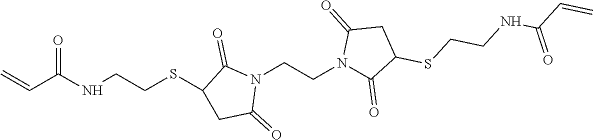 [SMILES: C=CC(=O)NCCSC1CC(=O)N(CCN2C(=O)CC(SCCNC(=O)C=C)C2=O)C1=O] |
| 7 | 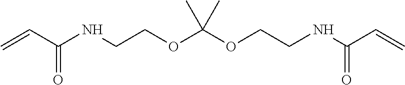 [SMILES: C=CC(=O)NCCOC(C)(C)OCCNC(=O)C=C] |
| 8 | 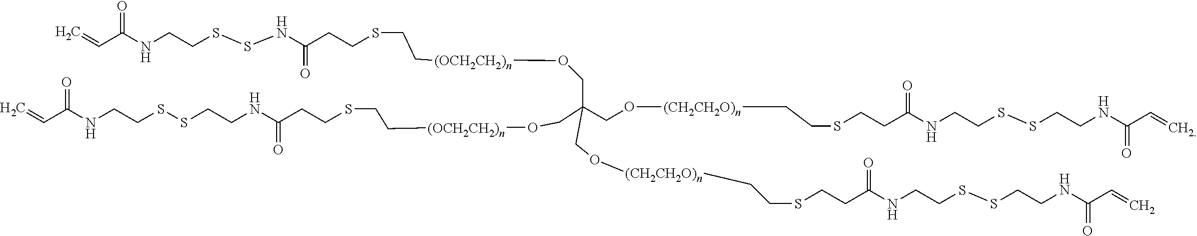 In some cases, each instance of n is independently an integer selected from about 1 to about 500. |
| 9 | 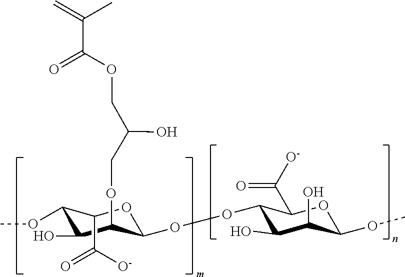 In some cases, n and m are independently integers selected from about 1 to about 500, wherein at least one of n and m is at least 1. In some cases, n and m are each at least 1. |
| 10 | 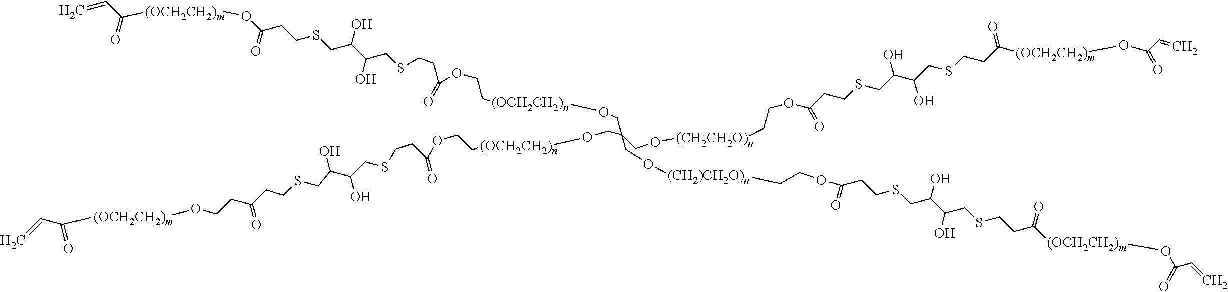 In some cases, each instance of n is independently an integer selected from about 1 to about 500. In some cases, each instance of m is independently an integer selected from about 1 to about 500. |
| 11 | 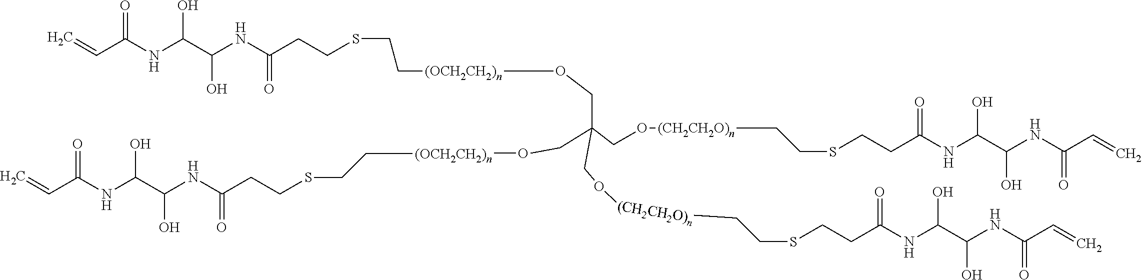 In some cases, each instance of n is independently an integer selected from about 1 to about 500. |
| 12 | 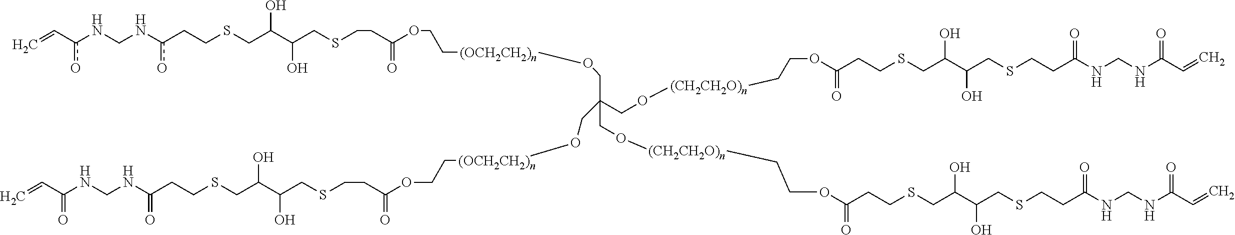 In some cases, each instance of n is independently an integer selected from about 0 to about 500. |
| 13 | 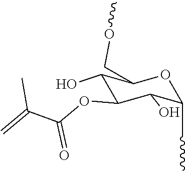 In some cases, structure 13 comprises from about 1 to about 500 monosaccharide units. |
| 14 | 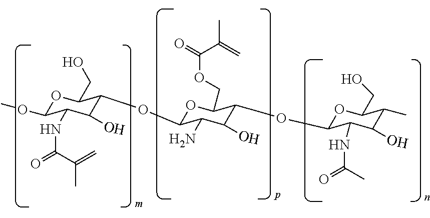 In some cases, n, m, and p are independently integers selected from about 1 to about 500, wherein at least one of n, m, and p is at least 1. In some cases, each of n, m, and p are at least 1. |
| 15 | 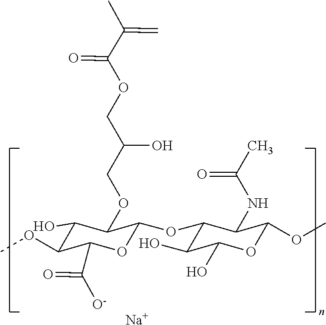 In some cases, n is an integer selected from about 1 to about 500. |
| 16 | 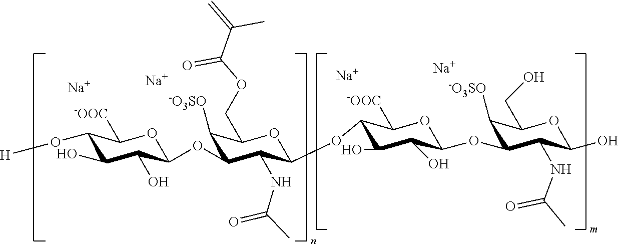 In some cases, n and m are independently integers selected from about 1 to about 500, wherein at least one of n and m is at least 1. In some cases, n and m are each at least 1. |
| 17 | 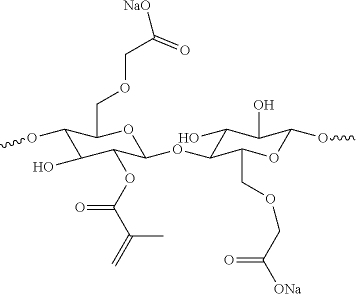 In some cases, structure 17 includes from about 1 to about 500 monosaccharide units. |
| 18 | 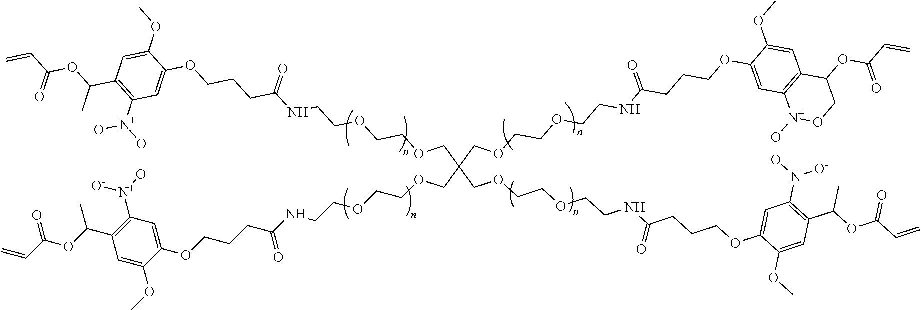 In some cases, each instance of n is independently an integer selected from about 1 to about 500. |
| 19 | 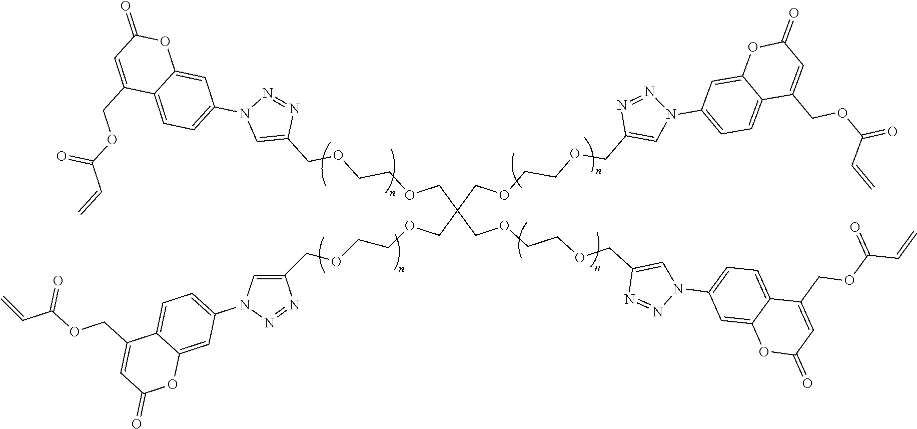 In some cases, each instance of n is independently an integer selected from about 1 to about 500. |
| 20 | 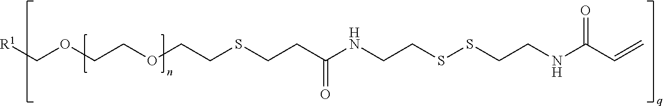 wherein R¹ is: i) a C₁ alkyl group, ii) a C₂-C₁₈ linear or branched saturated alkyl group, or iii) a C₃-C₈ cyclic saturated alkyl group substituted with 0-4 independently selected C₁-C₃ alkyl groups; wherein R¹ is substituted with q instances of 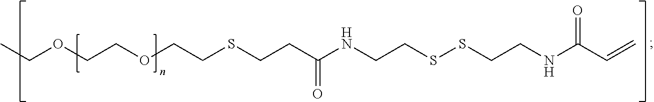 the remaining substituents on R¹ are hydrogen, q is an integer from 2 to 32, and each instance of n is an integer selected from about 1 to about 500. |
| 21 | 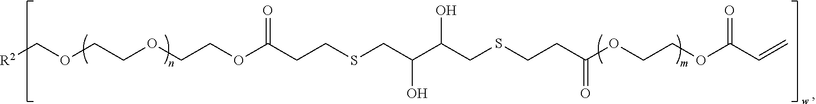 wherein R² is: i) a C₁ alkyl group, ii) a C₂-C₁₈ linear or branched saturated alkyl group, or iii) a C₃-C₈ cyclic saturated alkyl group substituted with 0-4 independently selected C₁-C₃ alkyl groups; wherein R² is substituted with w instances of 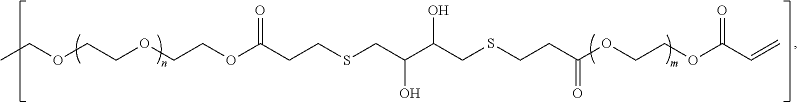 the remaining substituents on R² are hydrogen, w is an integer from 2 to 32, each instance of n is an integer independently selected from about 1 to about 500, and each instance of m is an integer independently selected from about 1 to about 500. |
| 22 | 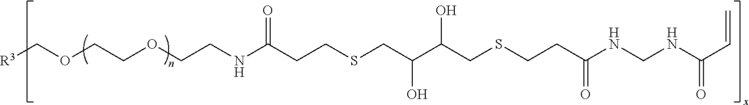 wherein R³ is: i) a C₁ alkyl group, ii) a C₂-C₁₈ linear or branched saturated alkyl group, or iii) a C₃-C₈ cyclic saturated alkyl group substituted with 0-4 independently selected C₁-C₃ alkyl groups; wherein R³ is substituted with x instances of 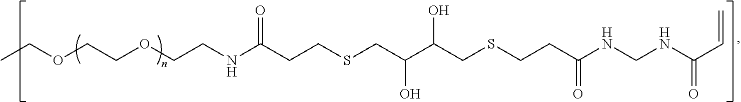 remaining substituents on R³ are hydrogen, x is an integer from 2 to 32, and each instance of n is an integer independently selected from about 1 to about 500. |
| 23 | 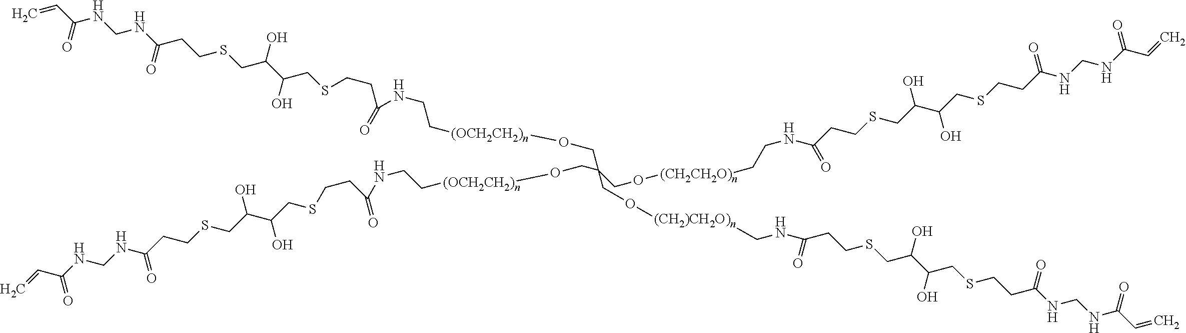 In some cases, each instance of n is independently an integer selected from about 1 to about 500. |
| 24 | 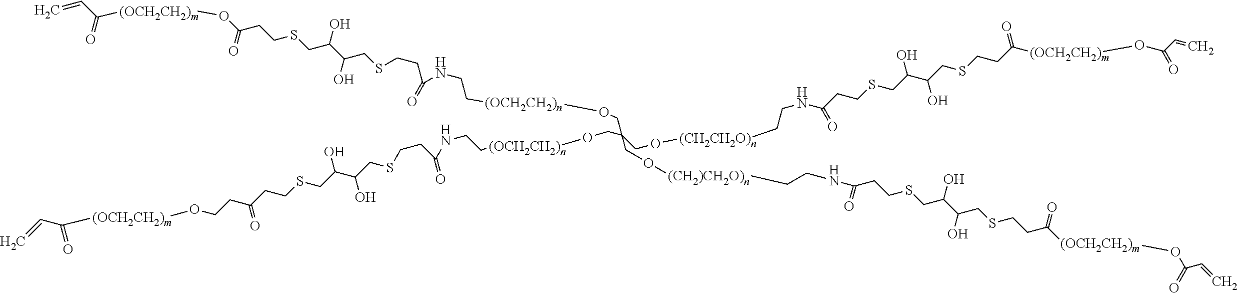 In some cases, each instance of n is independently an integer selected from about 1 to about 500. In some cases, each instance of m is independently an integer se- lected from about 1 to about 500. |
| 25 | 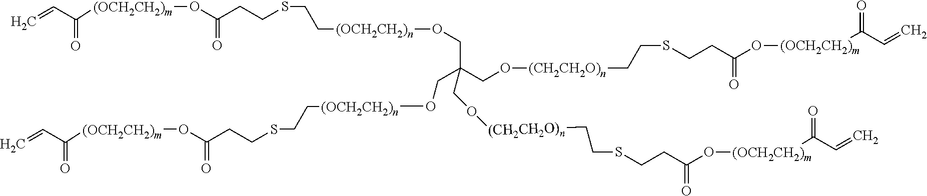 In some cases, each instance of n is independently an integer selected from about 1 to about 500. In some cases, each instance of m is independently an integer selected from about 1 to about 500. |
| 26 | 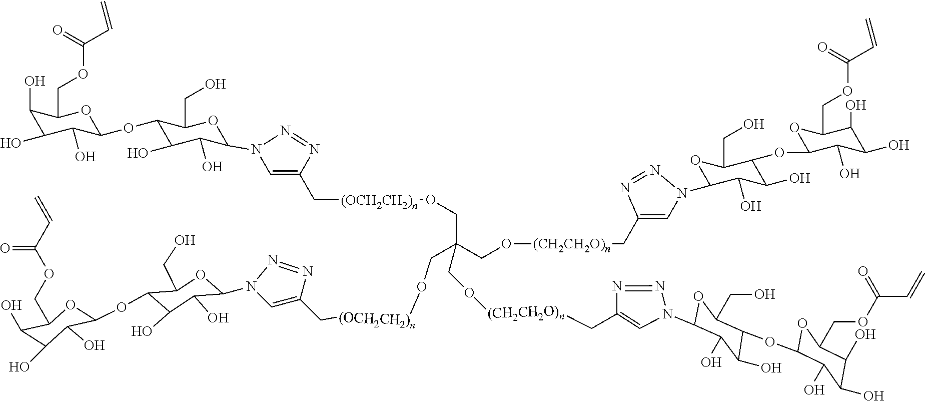 In some cases, each instance of n is independently an integer selected from about 1 to about 500. In some cases, each instance of m is independently an integer se- lected from about 1 to about 500. |

A polymer precursor can further comprise additional reagents that affect polymerization and polymer matrix properties. As examples, a polymer precursor can include a crosslinker, a porogen, a viscosity-modifying agent, an acid, a base, a catalyst, a salt, a photoinitiator, or a combination thereof. As another example, polymer precursors can include a crosslinker, a porogen, a photoinitiator, and a buffer.

As used herein, the term “crosslinker” denotes a species with two or more polymerizable groups. For example, where the polymerizable group is an ethylenically unsaturated group, a crosslinker would contain two or more ethylenically unsaturated groups. In another example, a crosslinker can contain three or more reactive centers for bifunctional polymer synthesis (e.g., the three methoxy groups of trimethoxybenzene in the context of polyester synthesis).

As used herein, the term “porogen” can denote a species that modulates the porosity of a polymer matrix. A porogen can be dispersed with the reactants before the polymerization process of forming the polymer matrix. Porogens typically diffuse out of polymer matrices following polymerization, leaving pores in the regions that they occupied. Porogen size, concentration, hydrophobicity, and hydrophilicity can thus influence pore density and pore size in polymer matrices. Examples of porogens consistent with the present disclosure include particles (e.g., polymeric, ceramic, metal, metal oxide, or hydrogel particles), polymers such as polyethylene glycol and alginate, and vesicles such as liposomes or micelles.

As used herein, the term “photoinitiator” can denote a species that generates a radical upon photoexcitation. In many cases, a photoinitiator included in a polymer precursor formulation is a type I photoinitiator, that is a molecule that generates radicals through intramolecular cleavage (e.g., homolysis) upon photoexcitation, or a type II photoinitiator, that is a molecule that abstract an electron or hydrogen atom from a co-initiator following photoexcitation. Examples of photoinitiators utilizable in the present methods include acetophenone, anisoin, anthraquinone, anthraquinone-2-sulfonic acid, benzil, benzoin, benzophenone, 3,3′,4,4′-benzophenonetetracarboxylic dianydride, 4-benzoylbiphenyl, 2-benzyl-2-(dimethylamino)-4′-morpholinobutyrophenone, dibenzosuberenone, 2,2-diethoxyacetophenone, 2-ethylanthraquinone, ferrocene, 2-isopropylthioxanthone, lithium phenyl (2,4,6-trimethylbenzoyl) phosphinate, methyl-2-benzoylbenzoate, and thiooxanthen-9-one.

### Nucleic Acid Barcodes

In some cases, one or more surfaces of a fluidic device (e.g., first surface 101, second surface 102, or first and second surfaces 101 and 102 of channel 100 of FIG. 1) may contain one or more nucleic acid barcodes (e.g., nucleic acid barcodes 722 of FIGS. 7A-B, nucleic acid barcodes 822 of FIGS. 8A-B and FIG. 10). One or more surfaces may contain, for example, about 1 to about 50,000,000 barcodes. The barcodes or subsets of the barcodes may be different from one another (e.g., each barcode may be unique). In some cases, a barcode (or a portion/subset of barcodes) may be associated with the location of the barcode on a surface (location coordinates (e.g., x-, y-coordinates) on a surface of a channel). In particular, a barcode may include a sequence that is unique (“spatially-addressed”) to a region or discrete spot along a surface of the fluidic device. Each region or discrete spot may contain a plurality of nucleic acid barcodes that share a common “spatially-addressed” sequence, while different discrete spots may be associated with different spatially-addressed sequences. Accordingly, the spot or region of origin of a barcode may be determined based on its spatially-addressed sequence. A discrete spot or region that contains barcodes with common “spatially-addressed” sequences may cover an area, for example, of about 10 to about 10⁵ μm² along the first or second surface of the fluidic device. The first or second surface of the fluidic device may contain about 10 to about 10⁷ discrete spots. In exemplary embodiments, a surface of a channel within a fluidic device may include about 5×10³ to about 5×10⁴ discrete spots. In further embodiments, a surface of a channel within a fluidic device may include about 5 to about 10² discrete spots per square millimeter. or regions that contain barcodes with unique “spatially-addressed” sequences along one or more surfaces. Similarly, the fluidic device may comprise one or more fluidic channels, each of which may include about 50 to 10⁷ spots or regions that contain barcodes with unique “spatially-addressed” sequences or spatially-addressed sequences that are associated with a region or discrete spot within each channel.

### Cellular and Biomolecular Analysis

“Cells” that may be cultured and assayed by methods and systems described herein may comprise any biological cells including, but not limited to, vertebrate, non-vertebrate, eukaryotic, mammalian, microbial, protozoan, prokaryotic, bacterial, archaea, insect, or fungal cells. In some embodiments, mammalian cells are assayed by methods and systems described herein. In particular, any population of mammalian cells which may be, or have been, induced, treated, modified or genetically altered (i.e. genetically engineered) for use in a medical, industrial, environmental, or remedial process, may be analyzed by methods and systems described herein. In some embodiments, “cells” as used herein comprise genetically modified cells. In some embodiments, “cells” comprise stem cells that have been induced to differentiate. In some embodiments, “cells” refer to cells modified by CRISPR Cas9 techniques. In some embodiments, “cells” refer to cells of the immune system including, but not limited to, cytotoxic T lymphocytes, regulatory T cells, CD4+ T cells, CD8+ T cells, natural killer cells, antigen-presenting cells, or dendritic cells. In some embodiments, “cells” refer to diseased cells such as cancer cells or virally infected cells.

An aptamer-based cellular analysis method can include one or more additional forms of cellular analysis. Such analyses may optionally be performed on a cell enclosed in a chamber, and may be performed in real-time or at defined timepoints. Accordingly, a single cell that is enclosed within a chamber within a fluidic device can be tracked independently of other cells that are present in the fluidic device. The aptamer-based analysis and one or more additional cellular analysis methods may be performed sequentially or in tandem. One or more cellular characteristics determined in a method can be selected from cytotoxicity, proliferative capacity, proliferation rate, activation status, cellular identity, purity, gene expression profile, transcriptome, surface marker expression, soluble factor secretion, activation status, epigenetic profile, sequence copy number (e.g., integrated viral copy number for transduced cells, plasmid copy number for transiently transfected cells, or gene copy number), or a combination thereof. Additional assays may include culture contamination assays including, but not limited to, viral, bacterial, yeast, mold, or mycoplasma assays, endotoxin assays, and cellular morphology assays. An assay can include one or more assay components, which may be provided with or as part of a channel include, but are not limited to, capture elements such as nucleic acid barcodes (which may include capture sequences as disclosed elsewhere herein), primers for captured nucleic acid amplification, antibodies, and detectable labels. In some embodiments, such assay components may be attached to a surface of a fluidic device or a polymer matrix wall exclusively, or on combinations of such surfaces, either exclusively, or in combination with other reagents. In some embodiments, assay components may be provided after synthesizing gel chambers. However, assay components may be premixed with cells prior to introduction into a fluidic device, added to a fluidic device simultaneously with cells, added to a fluidic device after the addition of cells to the fluidic device, added to a fluidic device before the introduction of cells to the fluidic device, or a combination thereof. Assay components include, but are not limited to, lysing reagents, transcription reagents, reverse transcription reagents, antibodies, polymerases, primers, beads, and the like. In some embodiments, cellular or assay components may be attached or captured by capture elements on a polymer matrix wall.

(i) gRNA Analysis

A method disclosed herein can include detecting a guide ribonucleic acid (gRNA) associated with a genetic modification of the cell (i.e., gRNA may be the cellular nucleic acid released by the cell, captured on a nucleic acid barcode, and sequenced). The cell can be transiently or stably transfected with a sequence encoding a gRNA specific for a particular genomic sequence. The guide RNA can be coupled to a barcode, an exogenous messenger RNA (e.g., a selection marker), a capture sequence (e.g., a poly A tail), or a combination thereof. The first cell or the aggregate of the first cells can express a Cas protein that can utilize the gRNA. Alternatively, a Cas protein can be delivered to the cells, for example in a chitosan particle or liposome. Cell growth, movement, or other characteristic or characteristics can then be correlated with a genomic edit imparted by a particular gRNA sequence. In one such method, the cells can be lysed to release guide RNA, the guide RNA can optionally be captured on a nucleic acid barcode, and then be used as a template for generating a cDNA molecule comprising a complement of the guide RNA sequence, and optionally additional sequences coupled to the guide RNA such as the exogenous mRNA, the barcode, or a combination thereof. The cDNA molecule can be coupled to a spatial location tag corresponding to a unique location within the channel of the fluidic device.

### (ii) Proliferation Rate

In some aspects, determining a characteristic of the one or more cells includes determining a proliferation rate. It is understood that the term “proliferation rate” may include a measure of a lack of proliferation. Proliferation rate can be determined by counting cells at least partially enclosed by the one or more chambers generated during an assay. For example, the one or more cells can be counted periodically (e.g., with fluorescence or brightfield imaging) following at least partial enclosure within the one or more chambers to determine a rate of change in the number of cells. Separate proliferation rates can be determined for each cell or collection of cells enclosed by a unique chamber or collection of chambers. In some embodiments, cells may be stained with a membrane or intracellular dye for determining proliferation by dye dilution so that an independent measure of cell proliferation may be obtained. Exemplary intracellular dyes for dye dilution include, but are not limited to, Hoechst 33342, carboxyfluorescein succinimidyl ester (CFSE), and the like. After counts are recorded for each chamber, further assays may be conducted on the clonal populations within the chambers to identify the cell types, for example, by an assessment of cell surface proteins, cell protein secretions, transcriptome, or the like.

(iii) Soluble Factor Analysis

In some aspects, determining a characteristic of the one or more cells includes detecting a soluble factor secreted by the one or more cells. As detailed elsewhere herein, in some cases, secreted soluble factors are detected with aptamers coupled to a surface of a fluidic device. However, in other cases, the soluble factors are detected with other binding agents such as antibodies. One challenge for studying soluble factors is that these species typically diffuse throughout fluidic devices, thereby preventing measurements on single cells. Furthermore, soluble factors are often removed from a fluidic device during media refreshment, preventing soluble factor quantitation. The fluidic devices and methods of the present disclosure, which allow cells to be maintained under quiescent conditions for extended periods of time, facilitate soluble factor capture on or adjacent to individual cells over extended periods of time, enabling higher accuracy and sensitivity for soluble factor detection.

Soluble factor analysis can include disposing a capture surface (e.g., a bead) comprising an affinity reagent (e.g., an aptamer or an antibody) that binds the soluble factor adjacent to one or more cells and detecting the soluble factor bound to the capture surface. Disposing the capture surface adjacent to the one or more cells can denote enclosing or at least partially enclosing the capture surface with the one or more cells within the one or more chambers, and optionally removing non-enclosed capture surfaces from the channel of the fluidic system. The capture surface can be loaded into the channel at a controlled density, for example 1 capture surface per about 100, 50, 10, 5, 1, 0.5, 0.1, 0.05, 0.01, 0.005, or 0.001 mm² of the channel.

In an exemplary embodiment, the capture surface comprises a bead. As used herein, the term “bead” can denote a microparticle or a nanoparticle, such as a ceramic, metal, metal oxide, polymer, or saccharide-based 30 to 10000 μm particle. However, further capture surfaces, including nanotubes, nucleic acid nanostructures, and antibody Fc domains. The capture surface affinity reagent can, as non-limiting examples, include antibodies, antibody fragments, aptamers, affimers, or a combination thereof.

Soluble factor detection may be performed with a bispecific binding agent capable of simultaneously binding to a cell and to a soluble factor secreted by the cell. The bispecific binding agent can be coupled to a target cell of interest and then used to capture soluble factors secreted by the cell. In this way, the bispecific binding agent may couple the soluble factor to the surface of the cell. The soluble factor may then be detected, for example by coupling a detectable binding agent such as a fluorescent antibody to the soluble factor coupled to the surface of the cell, and measuring the detectable binding agent. Unlike antibody modified beads that reside randomly within the chamber for capturing secretions that happen to diffuse to the beads, bispecific antibodies are coupled to the cell surface allowing for a more efficient capture of secretions emanated from the cell surface.

In a further exemplary embodiment, the soluble factor comprises a cytokine such as interferon-γ (IFN-γ) and interferon-a (IFN-α), an interleukins such as interleukin-1 (IL-1), interleukin-2 (IL-2), interleukin-4 (IL-4), interleukin-5 (IL-5), interleukin-6 (IL-6), interleukin-7 (IL-7), interleukin-10 (IL-10), interleukin-13 (IL-13), interleukin-15 (IL-15), interleukin-21 (IL-21), or interleukin-23 (IL-23), a colony stimulating factor (CSFs) such as granulocyte-macrophage colony stimulating factor (GM-CSF), granulocyte colony stimulating factor (G-CSF), or a tumor necrosis factors (TNF) such as TNF-α or TNF-β. In another embodiment, the secreted factor comprises an effector molecule such as a granzyme.

A soluble factor bound to a capture surface (e.g., an affinity reagent of a capture surface) can be detected by contacting the soluble factor bound to said capture surface with a labeled antibody configured to bind to the soluble factor, and detecting the labeled antibody. Multiple soluble factors can be detected in a single assay by providing a capture surface or plurality of capture surfaces that comprise a plurality of affinity reagents configured to bind the plurality of soluble factors, contacting the plurality of soluble factors bound to the capture surface or plurality of capture surfaces with a plurality of labeled antibodies configured to bind to the plurality of soluble factors, and detecting a plurality of labels coupled to the plurality of antibodies.

### (iv) Transcriptomic Analysis

In some aspects, determining a characteristic of the one or more cells includes sequencing at least a portion of a transcriptome of the one or more cells. As disclosed elsewhere herein, a fluidic device may contain nucleic acid barcodes that are configured to capture both mRNA from cells and aptamers input into the fluidic device for cellular analysis. Alternatively, a fluidic device may contain a first type of nucleic acid barcode that is configured to capture mRNA from cells and a second type of nucleic acid barcode that is configured to capture aptamers. The first and second types of nucleic acid barcodes can be disposed within a common discrete spot along the surface of the fluidic device.

A method for tandem surfaceome and transcriptomic analysis can include co-enclosing a cell with nucleic acid barcodes in one or more chambers, wherein the aptamers are coupled to biomarkers on the surface of the cell. The aptamers and mRNA can be released from the cell, for example through lysis, to diffuse to and then couple to the nucleic acid barcodes in the one or more chambers. The one or more chambers may have pores that are smaller than the mRNA and aptamers, and may thereby prevent mRNA and aptamers from entering or leaving the chamber. The one or more chambers may optionally be degraded after the mRNA and aptamers couple to the nucleic acid barcodes in the chamber. The mRNA and aptamers can be extended using the nucleic acid barcodes as a template, and the nucleic acid barcodes may be extended using the mRNA and aptamers as templates as described elsewhere herein. The resultant extended nucleic acid barcodes, extended aptamers, and/or extended mRNA may then be sequenced.

The nucleic acid barcodes may contain spatial barcode sequences that are uniquely associated with the cell and/or the one or more chambers. Accordingly, extended nucleic acid barcodes (which contain the spatial barcodes) and aptamers and mRNA extended using the nucleic acid barcodes as templates (which contain complements of the spatial barcodes) may be associated with the cell or the one or more chambers. The nucleic acid barcodes may also contain unique molecular identifiers to facilitate mRNA and aptamer quantitation by normalizing sequencing counts of extended nucleic acid barcodes, aptamers extended using the nucleic acid barcodes as templates, and/or mRNA extended using the nucleic acid barcodes as templates. For example, the number of instances of each mRNA sequence or aptamer may be determined based on the number of unique molecular identifier sequences associated with that mRNA sequence or aptamer.

In some cases, mRNA, aptamer, and/or nucleic acid barcode extension involves reverse transcription. Reverse transcription reagents may comprise conventional reagents for reverse transcription; namely, a reverse transcriptase (such as, a Moloney murine leukemia virus (MMLV)), dNTPs, optional RNase inhibitor, buffer.

The sequencing step may be carried out at the sites of the captured mRNAs (in situ) or cDNAs may include a spatial barcode and be eluted and sequenced on a separate sequencing instrument (“external” sequencing). For in situ sequencing, further steps may include (i) amplifying the complementary DNAs, e.g. by bridge amplification, or like method, (ii) sequencing the amplified complementary DNAs, e.g. by a sequencing-by-synthesis technique, and (iii) determining relative expression of the mRNAs for the cells of each of the chambers. For external sequencing, further steps may include (i) providing capture elements comprising spatial barcodes, (ii) synthesizing cDNAs comprising spatial barcodes, and (iii) eluting and sequencing the cDNAs and correlating each cDNA with a chamber location by its spatial barcode.

### (v) Cytotoxicity

In a further aspect, the one or more cells comprises an effector cell, and determining the characteristic of the one or more cells comprises measuring a cytotoxicity of the effector cell. Such a method may be performed by at least partially enclosing one or more target cells with the effector cell, and counting dead cells, viable cells, or a combination thereof from among the one or more target cells. As non-limiting examples, the target cells may comprise a sample of tumor cells of a patient, or target cells may be from a cell line, e.g. tumor cell line, such as, hepatic tumor cell line, SK-HEP-1, Chava et al, J. Vis. Exp., 2020 Feb. 22: (156): 10.3791/60714. Examples of effector cells include Tc1 cells, Tc2 cells, Tc9 cells, Tc17 cells, Tc22 cells, natural killer cells. In some embodiments, the effector cell is engineered for a therapeutic purpose. For example, the effector cell may express a chimeric antigen receptor that confers cytotoxicity against a particular cancer.

A cytotoxicity assay may include loading the effector cell and target cells into the channel (either simultaneously or sequentially). In some aspects, the target cells are loaded into channel where they optionally adhere to a surface prior to effector cell loading. In other aspects, the effector cell is loaded into the channel prior to target cell loading. The effector cell may be at least partially enclosed with the target cells inside one or more chambers. The target cells and effector cells are then incubated. The incubation may be performed in the presence of a vital dye that generates an optical signal in response to a characteristic of viable cells or dead cells to facilitate dead cell enumeration. The method may include counting the number of target cells killed per effector cell. The method may also include counting the number of target cells killed per unit time. Alternatively or in addition thereto, the target cells may be pre-treated with a dye that internalizes into the target cells prior to loading into the channel. In addition to target cell killing, effector cells can be identified based on other cellular characteristics disclosed herein, such as their secretome, transcriptome, or the like.

### (vi) Cellular Activation

In some aspects, the characteristic of the one or more cells comprises activation. Cellular activation can be detected using numerous assays disclosed herein, including surface marker expression, soluble factor secretion, transcriptomic analysis, proliferation or changes in proliferation, changes in morphology, change in cytotoxicity, or a combination thereof. As non-limiting examples, these methods are broadly amenable to detecting activation caused by contact between a second cell and a cell of the one or more cell, a soluble factor secreted by the second cell and the cell of the one or more cells, and combinations thereof. In a particular aspect of the present disclosure, determining activation comprises detecting a surface marker of the one or more cells. For example, the method can comprise contacting the one or more cells with a binding agent configured to bind to the surface marker and detecting the binding agent.

### Fluidic and Optical Systems

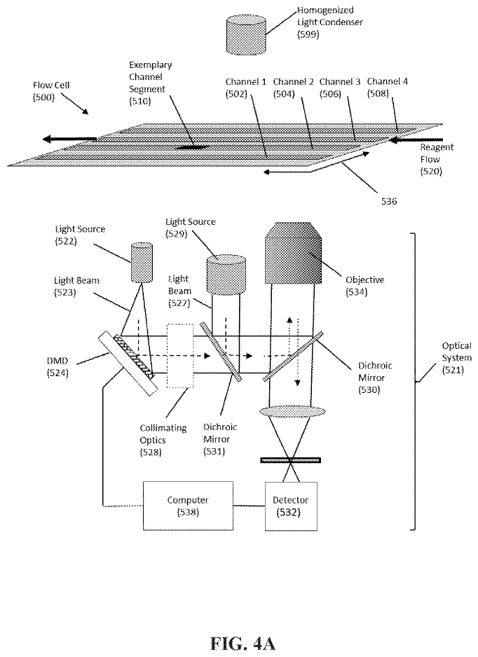

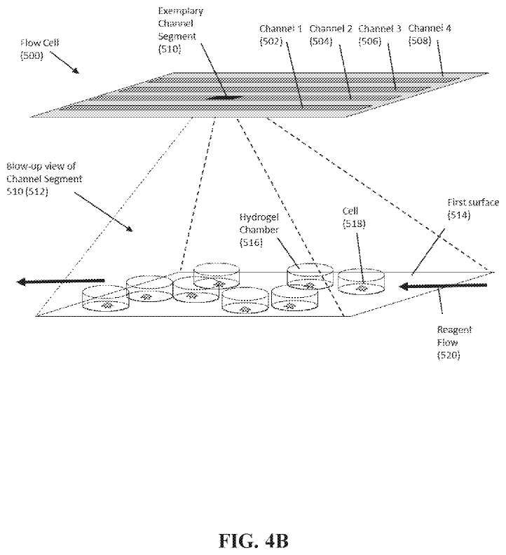

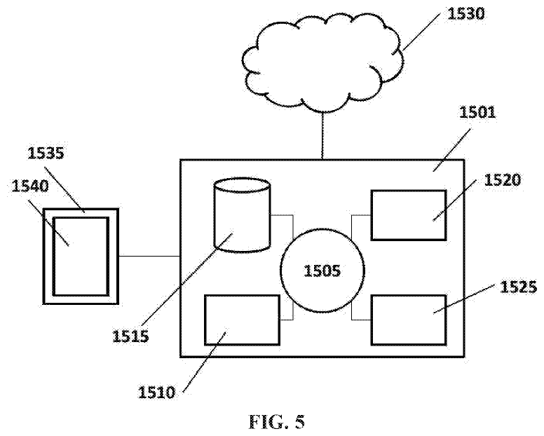

FIG. 4A is an example of a system for carrying out the disclosed methods. Flow cell (500) is a component of a fluidic device that provides channels for carrying out a variety of assays and liquid handling components under programmable control for delivering samples and reagents to the channels. In this illustration, four channels (502, 504, 506, and 508) are shown. However, as detailed elsewhere herein, systems of the present disclosure can utilize flow cells with fewer or greater numbers of channels.

The system of FIG. 4A includes an optical system (521) for photosynthesizing chambers at locations of cells or other analytes in the channels (502, 504, 506, and 508) of the flow cell (500) and for collecting images and other optical signals. The optical system (521) includes a light source (522) that generates a light beam (523) of appropriate wavelength light (e.g. UV light) for synthesizing chambers (e.g., hydrogel chambers) in the flow cell (500). The light beam (523) that passes through an appropriate photo-mask or beam-shaping or beam steering (Galvo) system (524) for shaping a beam to synthesize a desired structure or structures in a channel. For example, the beam shaping system (524) may project circular patterns of light from the light source (522) onto the flow cell (500) to cause a polymer precursor to photopolymerize in the shape of a cylindrical chamber (e.g., chamber 516 in FIG. 4B). In some embodiments, this beam shaping system (524) includes a digital micromirror device (DMD). In other embodiments, a physical photo-mask may be employed. Reflected light from DMD (524) is shaped using conventional optics, e.g. collimating optics (528), and is directed through objective lens system (534) into channel 2 segment (510). In other embodiments, the beam shaping system (524) can include a virtual mask (e.g., a computer code or a digital system). The virtual mask may render one or more electrodes, or arrays of electrodes, to produce spatially-modulated energy (e.g., light, electrical current, etc.) to form the patterned polymer matrix in the flow cell (500). The virtual mask may also block a portion of light from the light source (522). When a virtual mask is utilized in this manner, the virtual mask can prevent light from being emitted to a location where analyte (e.g., a cell 518) is present within the flow cell (500). In exemplary embodiments, the light is directed by one or more dichroic mirrors (530 and 531).

Chamber position, shape and polymer matrix wall thickness is determined at least in part from cell position information determined from images collected by detector (532). Objective (534) and flow cell (500) move relative to one another in the xy-directions (536) to photosynthesize chambers at any position in any of the channels. In some embodiments, the flow cell (500) moves and optical system (521) is stationary. The system may utilize light from a light source (599), such as a homogenized light condenser, that is positioned on an opposite side of the flow cell as the optical system and directs light through the flow cell (500) to the objective (534). To achieve this functionality, the light source positioned on the opposite side of the flow cell (599) can be configured to move in tandem with the optical system (521), or the light source (599) and optical system can be stationary and the flow cell (500) can be moved to the region illuminated by the light source (599) and from which light is collected by the objective (534). In some embodiments, objective (534) may also direct light beam (527) from light source (529) to targets, such as cells, on first surface (514, see FIG. 4B) and collect optical signals, such as fluorescent signals, from assays taking place on first surface (514). Optical signal collection can also be carried out with a separate objective. Information collected by detector (532), particularly cellular positions in their respective channels, is employed by computer (538) and/or subsidiary controllers to direct DMD (524) and translation devices controlling the relative positions of objective (534) and flow cell (500) to synthesize hydrogel chambers of the appropriate shape and size at the appropriate locations.

FIG. 4B provides a blown-up view of the exemplary channel segment (510) of the flow cell of FIG. 4A. On first surface (514) of channel 2 (504) a plurality of cells, e.g. (518), are each enclosed by a hydrogel chamber, e.g. (516).

One of ordinary skill in the art would recognize that optical systems with different configurations than those of FIGS. 4A and 4B may be employed for carrying out these functions. In some embodiments, a plurality of DMD-objective subsystems for synthesizing hydrogel structures may be employed to increase the speed of synthesis by synthesizing multiple structures simultaneously.

### Computer Systems

The present disclosure provides computer systems that are programmed to implement methods of the disclosure. FIG. 5 shows a computer system 1501 that may be programmed or otherwise configured to perform methods described herein. The computer system 1501 can regulate various aspects of the present disclosure, such as, for example, identifying a biological component, detecting a barcode, controlling a spatial light modulator to shape a light beam, providing energy from an energy source, or detecting or measuring a local parameter using a sensor. The detector may be a camera (e.g., a fluorescent camera), such as a charged coupled device (CCD) camera capable of collecting optical signals and position information from a plurality of sources distributed over a planar region. The computer system 1501 can be an electronic device of a user or a computer system that may be remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that may be in communication with the Internet. The network 1530 in some cases may be a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit may be an application specific integrated circuit (ASIC).

The storage unit 1515 can store files, such as drivers, libraries, and saved programs. The storage unit 1515 can store user data, e.g., user preferences and user programs. The computer system 1501 in some cases can include one or more additional data storage units that are external to the computer system 1501, such as located on a remote server that may be in communication with the computer system 1501 through an intranet or the Internet.

The computer system 1501 can communicate with one or more remote computer systems through the network 1530. For instance, the computer system 1501 can communicate with a remote computer system of a user (e.g., a laptop, a personal computer, a tablet, or a mobile phone). Examples of remote computer systems include personal computers (e.g., portable PC), slate or tablet PC's (e.g., Apple® iPad, Samsung® Galaxy Tab), telephones, Smart phones (e.g., Apple®iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that may be carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, an image of a biological component, a barcode, a signal or measurement of a local parameter. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, identify a biological component, detect a barcode, generate a spatial modulating element (e.g., a mask), provide energy from an energy source, detect or measure a local parameter using a sensor, etc.

### EXAMPLES

### Example 1

### Aptamer Capture and Reverse Transcription Using Surface-Bound Nucleic Acid Barcodes

This example is directed to aptamer capture efficiency on nucleic acid barcodes coupled to a surface of a fluidic device. The fluidic device included eight parallel fluidic channels with nucleic acid barcodes printed on their top surfaces. The nucleic acid barcodes were arranged in arrays of discrete spots, with the nucleic acid barcodes within each discrete spot sharing a spatial barcode sequence uniquely associated with that spot. The nucleic acid barcodes were coupled to the top surfaces at their 5′-ends and contained poly(T) sequences at their 3′ ends for poly(A) capture.

In a first step, the fluidic device was preconditioned with 90 μL of DPBSM, which was flowed through each lane and incubated at 37° C. for 10 minutes. Following incubation, each lane was washed three times with 200 μL of DPBS (1×) at room temperature.

Next, an aptamer pool consisting of ten distinct poly(A)-tailed aptamers was input into the fluidic channels. Sequences of the ten aptamers are provided in Table 2. Each aptamer shared common 5′ 20mer sequences and 3′ poly(A) sequences with 33-50 nucleotide aptamer binding portions disposed therebetween. The aptamer pool contained 0.1 μM of each of the ten aptamers, corresponding to a total aptamer concentration of 1 μM. The aptamers were contained in DPBSM supplemented with salmon sperm DNA. The fluidic device was incubated at room temperature for 45 minutes to allow the poly(A) siaptamer tails to hybridize to poly(T) sequences of the nucleic acid barcodes. Each fluidic channel was then washed three times with 200 μL of DPBS (1×) at room temperature to remove aptamers not coupled to nucleic acid barcodes.

TABLE 2

| Aptamer | SEQ ID NO | SEQUENCE |
|---|---|---|
| Aptamer 1 | SEQ ID NO: 1 | CCTTGGCACCCGAGAATTCCGCATCTAACTGCTGCGC |
| | | CGCCGGGAAGAACTGTACGGTTAGATGCAAAAAAAA |
| | | AAAAAAAAAAAAAAAAAAAAAA |
| Aptamer 2 | SEQ ID NO: 2 | CCTTGGCACCCGAGAATTCCCAGCAGCGTAAAGGGG |
| | | GTGTTTGTGCGGTGTGGAGTGCGCGTGCTGCTGGAA |
| | | AAAAAAAAAAAAAAAAAAAAAAAAAAAA |
| Aptamer 3 | SEQ ID NO: 3 | CCTTGGCACCCGAGAATTCCATCAGGCTGGATGGTAG |
| | | CTCGGTCGGGGTGGGTGGGTTGGCAAGTCTGATAAA |
| | | AAAAAAAAAAAAAAAAAAAAAAAAAAA |
| Aptamer 4 | SEQ ID NO: 4 | CCTTGGCACCCGAGAATTCCGCCATTGCCATTGCCAT |
| | | TGCCATTGCCATTGCCATTGCCATTGCCATTAAAAAA |
| | | AAAAAAAAAAAAAAAAAAAAAAAA |
| Aptamer 5 | SEQ ID NO: 5 | CCTTGGCACCCGAGAATTCCCCAATACCGCGGGGTG |
| | | GGTCTAGTGTGGATGTTTAGGGGGCGGTATTGGAAAA |
| | | AAAAAAAAAAAAAAAAAAAAAAAAAA |
| Aptamer 6 | SEQ ID NO: 6 | CCTTGGCACCCGAGAATTCCTACAGGTTCTGGGGGG |
| | | TGGGTGGGGAACCTGTTAAAAAAAAAAAAAAAAAA |
| | | AAAAAAAAAAAA |
| Aptamer 7 | SEQ ID NO: 7 | CCTTGGCACCCGAGAATTCCCTAACCCCGGGTGTGGT |
| | | GGGTGGGCAGGGGGGTTAGAAAAAAAAAAAAAAAA |
| | | AAAAAAAAAAAAAA |
| Aptamer 8 | SEQ ID NO: 8 | CCTTGGCACCCGAGAATTCCTCGCCGCGTCTTTATGG |
| | | CTGGGGATGGTGTGGGTTGCGGCGAAAAAAAAAAA |
| | | AAAAAAAAAAAAAAAAAAAA |
| Aptamer 9 | SEQ ID NO: 9 | CCTTGGCACCCGAGAATTCCCTTATTCAATTCCTGTG |
| | | GGAAGGCTATAGAGGGGCCAGTCTATGAATAAGAAA |
| | | AAAAAAAAAAAAAAAAAAAAAAAAAAA |
| Aptamer | SEQ ID NO: 10 | CCTTGGCACCCGAGAATTCCCGCGCCGTACTAGATGC |
| 10 | | AACCCGACTACTAACGTCGTACGAGCGCGAAAAAAA |
| | | AAAAAAAAAAAAAAAAAAAAAAA |

In a third step, aptamer displacement was initiated using an invader oligonucleotide. A solution comprising 5 μM invader oligonucleotide in MgCl₂-free DPBS was introduced into each channel. The fluidic device was incubated at 37° C. for 20 minutes to permit displacement of weakly-bound aptamers from nucleic acid barcodes. Following incubation, each lane was washed three times with 200 μL of DPBS (1×) at room temperature.

Nucleic acid barcodes were then extended using bound aptamers as templates. The fluidic channels were first washed twice with 200 μL of NFB/PVSA. A reverse transcription (RT) reaction mixture with a strand-displacing reverse transcriptase was then loaded into each fluidic channel. Fluidic channel inlet and outlet ports were sealed to prevent evaporation, and the fluidic device was incubated at 42° C. for 90 minutes. Following incubation, each fluidic channel was washed twice with 200 μL of HT2 buffer and then treated with a single-stranded nucleic acid-specific exonuclease to degrade non-extended nucleic acid barcodes and unbound nucleic acids.

Extended nucleic acid barcodes were cleaved from the fluidic device surface and eluted from the fluidic device. Cleavage was performed using a mixture of uracil DNA glycosylase (UDG) and DNA glycosylase-lyase Endonuclease VIII, incubated in the fluidic channels at 37° C. for 60 minutes, to cleave uracil nucleotides disposed near 5′ ends of the extended nucleic acid barcodes. Eluate was collected from the fluidic channels and transferred into PCR tubes. Each fluidic channel was then washed with 55 μL, which was eluted and combined with the initial eluate. The total recovered volume was approximately. The samples collected from each channel were divided into three equal volume aliquots.

Eluted samples were then subjected to quantitative PCR (qPCR) analysis. Each analysis utilized 18 μL of eluate combined with 32 μL of PCR master mix. Two of the three aliquots obtained from each lane were used for amplification. Serial dilutions of each sample (1:10, 1:100, and 1:1000) were prepared to assess amplification efficiency and dynamic range. Each condition was analyzed using three dilution points and at least two technical replicates corresponding to different fluidic channels from the assay. Control samples with 1 nM and 0.1 nM aptamers were analyzed in parallel. Cq values from the qPCR amplification are summarized in Table 3. The relatively low Cq values indicated that aptamers could be captured on surface bound nucleic acid barcodes, challenged with invader nucleic acids, reverse transcribed, and amplified to levels suitable for sequencing.

TABLE 3

| Sample | Cq Value |
|---|---|
| No Dilution | 8.19 |
| No Dilution | 8.11 |
| 1:10 Dilution | 11.47 |
| 1:10 Dilution | 11.22 |
| 1:100 Dilution | 14.68 |
| 1:100 Dilution | 14.54 |
| 1:1000 Dilution | 17.74 |
| 1:1000 Dilution | 17.73 |
| Control: 1 nM Aptamer | 7.01 |
| Control: 1 nM Aptamer | 6.62 |
| Control: 0.1 nM Aptamer | 11.14 |
| Control: 0.1 nM Aptamer | 11.40 |

### Example 2

### Aptamer-Based Cell Surface Protein Characterization

This example is directed to multiplexed cell surface protein characterization with a pool of aptamers targeted to distinct cell surface proteins. A pool of thirteen aptamers, each specific for a different cell surface protein, was prepared. The aptamers are listed in Table 4. Each aptamer included a common 20mer sequence at its 5′ end, a poly(A) sequence at its 3′ end, and a 33-50 nucleotide aptamer binding portion disposed therebetween. Each aptamer was coupled to a fluorophore. The aptamer pool was combined with six different human cell lines: a T lymphocyte leukemia cell line (Jurkat cells), a pulmonary adenocarcinoma cell line (A549 cells), a pre-B cell acute lymphoblastic leukemia cell line (NALM-6 cells), a breast cancer cell line (MCF-7 cells), a Burkitt's lymphoma B-lymphocyte cell line (Raji cells), and a non-small cell lung cancer cell line (H1975 cells). Unbound aptamers were separated from the cells. Then, the population of aptamers coupled to each cell was measured using with fluorescence. The results of these analyses are summarized in Table 5, wherein the entries indicate baseline-corrected fluorescence intensity.

TABLE 4

| Aptamer | | |
|---|---|---|
| Target | SEQ ID NO | Sequence |
| CD71 | SEQ ID NO: 2 | CCTTGGCACCCGAGAATTCCCAGCAGCGTAAAGGGG |
| (TfR) | | GTGTTTGTGCGGTGTGGAGTGCGCGTGCTGCTGGAA |
| | | AAAAAAAAAAAAAAAAAAAAAAAAAAAA |
| PTK7 | SEQ ID NO: 1 | CCTTGGCACCCGAGAATTCCGCATCTAACTGCTGCGC |
| | | CGCCGGGAAGAACTGTACGGTTAGATGCAAAAAAAA |
| | | AAAAAAAAAAAAAAAAAAAAAA |
| c-Met | SEQ ID NO: 3 | CCTTGGCACCCGAGAATTCCATCAGGCTGGATGGTAG |
| | | CTCGGTCGGGGTGGGTGGGTTGGCAAGTCTGATAAA |
| | | AAAAAAAAAAAAAAAAAAAAAAAAAAA |
| CD3 | SEQ ID NO: 5 | CCTTGGCACCCGAGAATTCCCCAATACCGCGGGGTG |
| | | GGTCTAGTGTGGATGTTTAGGGGGCGGTATTGGAAAA |
| | | AAAAAAAAAAAAAAAAAAAAAAAAAA |
| SSC | SEQ ID NO: 7 | CCTTGGCACCCGAGAATTCCCTAACCCCGGGTGTGGT |
| | | GGGTGGGCAGGGGGGTTAGAAAAAAAAAAAAAAAA |
| | | AAAAAAAAAAAAAA |
| CD49d: | SEQ ID NO: 10 | CCTTGGCACCCGAGAATTCCCGCGCCGTACTAGATGC |
| CD29 | | AACCCGACTACTAACGTCGTACGAGCGCGAAAAAAA |
| | | AAAAAAAAAAAAAAAAAAAAAAA |
| CD62L | SEQ ID NO: 9 | CCTTGGCACCCGAGAATTCCCTTATTCAATTCCTGTG |
| | | GGAAGGCTATAGAGGGGCCAGTCTATGAATAAGAAA |
| | | AAAAAAAAAAAAAAAAAAAAAAAAAAA |
| EpCAM | SEQ ID NO: 11 | CCTTGGCACCCGAGAATTCCCACTACAGAGGTTGCG |
| | | TCTGTCCCACGTTGTCATGGGGGGTTGGCCTGAAAA |
| | | AAAAAAAAAAAAAAAAAAAAAAAAAA |
| FGFR1 | SEQ ID NO: 8 | CCTTGGCACCCGAGAATTCCTCGCCGCGTCTTTATGG |
| | | CTGGGGATGGTGTGGGTTGCGGCGAAAAAAAAAAA |
| | | AAAAAAAAAAAAAAAAAAAA |
| PD-L1 | SEQ ID NO: 6 | CCTTGGCACCCGAGAATTCCTACAGGTTCTGGGGGG |
| | | TGGGTGGGGAACCTGTTAAAAAAAAAAAAAAAAAA |
| | | AAAAAAAAAAAA |
| Unknown | SEQ ID NO: 12 | CCTTGGCACCCGAGAATTCCTGCAACGGGGGGAGCG |
| Target | | GATGTCTGGGAAAACCGCGGGGTGCCCTCCGAGCAA |
| | | AAAAAAAAAAAAAAAAAAAAAAAAAAAA |
| CD4 | SEQ ID NO: 13 | CCTTGGCACCCGAGAATTCCCCAGAGTGACGCAGCA |
| | | CCACCACCGTACAATTTTTTCATTACCTACTCGGAAA |
| | | AAAAAAAAAAAAAAAAAAAAAAAAAAA |
| IGHM | SEQ ID NO: 14 | CCTTGGCACCCGAGAATTCCAACACCGTGGAGGATA |
| | | GTTCGGTGGCTGTTCAGGGTCTCCTCCCGGTGAAAA |
| | | AAAAAAAAAAAAAAAAAAAAAAAAA |

TABLE 5

| Aptamer | Cell Type | | | | | |
|---|---|---|---|---|---|---|
| Target | Jurkat | A549 | Nalm6 | MCF7 | Raji | H1975 |
| TFR | 54343 | 72872 | 12447 | 54543 | 12817 | 27789 |
| PTK7 | 3318 | 794 | 3230 | 572 | 33 | 780 |
| c-Met | 181 | 2743 | 547 | 729 | 4371 | 9445 |
| CD3 | 2879 | 379 | 163 | 5385 | 172 | 104 |
| SSC | 787 | 2622 | 534 | 1626 | 584 | 737 |
| CD49d:CD29 | 2858 | 2 | 578 | −83 | 61 | 37 |
| CD62L | 1330 | −117 | 45 | −193 | 141 | −31 |
| EpCAM | 383 | 1379 | 565 | 229 | 481 | 182 |
| FGFR1 | 144 | 418 | 107 | 480 | 75 | 70 |
| PD-L1 | 228 | 442 | 130 | 125 | 228 | 34 |
| unknown | 81 | 168 | 118 | 89 | 199 | 208 |
| CD4 | 6 | 168 | 94 | 125 | −19 | 29 |
| IGHM | −20 | −113 | 156 | −116 | 28 | −54 |

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

What is claimed is:

1. A method for cellular analysis comprising:
combining aptamers with a cell, wherein a first subset of the aptamers couple to the cell and a second subset of the aptamers do not couple to the cell;
separating the second subset of the aptamers from the cell;
inputting the cell and the first subset of the aptamers into a fluidic device, wherein the fluidic device comprises nucleic acid barcodes;
hybridizing capturable sequences of the first subset of the aptamers to the nucleic acid barcodes;
extending the nucleic acid barcodes to generate extended nucleic acid barcodes or extending the first subset of the aptamers to generate extended aptamers;
eluting the extended nucleic acid barcodes or the extended aptamers from the fluidic device; and
sequencing the extended nucleic acid barcodes or the extended aptamers.

2. The method of claim 1, further comprising associating the first subset of the aptamers with an identity for one or more surface proteins on the cell.

3. The method of claim 2, further comprising identifying the cell based on the sequencing of the first subset of the aptamers.

4. The method of claim 1, further comprising before the hybridizing, dissociating the first subset of the aptamers from the cell.

5. The method of claim 4, wherein the dissociating comprises:
i) lysing the cell,
ii) reducing an ionic strength of a solution surrounding the cell,
iii) increasing or reducing a pH of the solution surrounding the cell,
iv) inputting a chaotropic agent into the fluidic device,
v) inputting an organic solvent into the fluidic device,
vi) increasing a temperature of the fluidic device, or
vii) a combination thereof.

6. The method of claim 1, wherein the separating comprises removing the second subset of the aptamers from the fluidic device in one or more wash steps, wherein the first subset of the aptamers remain coupled to the cell during the one or more wash steps.

7. The method of claim 1, wherein the separating comprises prior to the inputting, centrifuging a composition comprising the cell and the aptamers, thereby generating a supernatant comprising the second subset of the aptamers and a pellet comprising the cell and the first subset of the aptamers coupled to the cell, and separating the supernatant from the pellet;

8. The method of claim 1, wherein the separating comprises prior to the inputting, coupling a magnetic particle to the cell, wherein the cell is coupled to the first subset of the aptamers, and magnetically separating the magnetic particle coupled to the cell from the second subset of the aptamers.

9. The method of claim 1, wherein the separating comprises prior to the inputting, filtering a composition comprising the cell and the aptamers, thereby generating a filtrate comprising the second subset of the aptamers and a residue comprising the cell and the first subset of the aptamers coupled to the cell.

10. The method of claim 6, wherein the wash steps are subsequent to the synthesizing.

11. The method of claim 1, wherein:
i) the combining and the separating are prior to the inputting;
ii) the combining and the separating are subsequent to the inputting, the aptamers are input into the fluidic device, and the separating comprises removing the second subset of the aptamers from the fluidic device; or
iii) the inputting is subsequent to the combining and prior to the separating, the aptamers are input into the fluidic device along with the cell, and the separating comprises removing the second subset of the aptamers from the fluidic device.

12. The method of claim 1, wherein the aptamers comprise:
i) the capturable sequences at 3′ ends of the aptamers, and
ii) primers at 5′ ends of the aptamers.

13. The method of claim 1, wherein the aptamers further comprise aptamer identity barcodes, wherein the aptamer identity barcodes identify the aptamers to which they are coupled.

14. The method of claim 1, wherein the nucleic acid barcodes comprise:
i) spatial barcode sequences associated with locations of the nucleic acid barcodes on a surface of the fluidic device,
ii) unique molecular identifier sequences,
iii) oligonucleotide capture sequences,
iv) chemically cleavable moieties,
v) enzymatically cleavable moieties,
vi) primer binding sites, or
vii) a combination thereof.

15. The method of claim 14, wherein the sequencing comprises sequencing the spatial barcode sequences or complements of the spatial barcode sequences, and wherein the method further comprises associating the first subset of the aptamers with the cell based on the spatial barcode sequences or complements of the spatial barcode sequences.

16. The method of claim 14, wherein the oligonucleotide capture sequences comprise polyT sequences, and wherein poly A sequences of the capturable sequences hybridize to the polyT sequences.

17. The method of claim 1, wherein the sequencing comprises quantifying aptamers of the first subset of the aptamers.

18. The method of claim 1, further comprising synthesizing one or more chambers that co-enclose the cell, the first subset of aptamers coupled to the cell, and one or more of the nucleic acid barcodes, wherein the nucleic acid barcodes are coupled to a surface of the fluidic device.

19. The method of claim 18, further comprising degrading the one or more chambers.

20. The method of claim 18, further comprising dissociating the first subset of the aptamers from the cell subsequent to the synthesizing.

21. The method of claim 18, wherein the separating is subsequent to the synthesizing, wherein the separating comprises flowing a liquid into the fluidic device causing the second subset of the aptamers in the one or more chambers to flow out of the one or more chambers through a pore in a wall of the one or more chambers while retaining the cell in the one or more chambers, wherein the cell is larger than the pore in the wall of the one or more chambers.

22. The method of claim 1, further comprising analyzing cellular nucleic acids from the cell, the analyzing comprising:
releasing the cellular nucleic acids from the cell;
capturing the cellular nucleic acids or fragments of the cellular nucleic acids on additional nucleic acid barcodes coupled to the surface of the fluidic device; and
i) extending the additional nucleic acid barcodes using the cellular nucleic acids as templates and sequencing the extended additional nucleic acid barcodes,
ii) extending the cellular nucleic acids using the additional nucleic acid barcodes as templates and sequencing the extended cellular nucleic acids, or
iii) a combination thereof.

23. The method of claim 22, wherein the cellular nucleic acids from the cell comprise mRNA.

24. The method of claim 22, wherein the cellular nucleic acids from the cell comprise guide RNA, wherein the guide RNA comprises a reporter sequence corresponding to a genetic edit for generating a surface protein from the cell, the method further comprising associating the aptamer coupled to the cell with an identity of the surface protein for the cell.
